(19) United States
(12) Patent Application Publication
(10) Pub. No.: US 2020/0171214 A1
(43) Pub. Date: Jun. 4, 2020

(54) BIORESORBABLE SURFACE COATING FOR DELAYING DEGRADATION

(71) Applicant: Drei Lilien PVG GmbH & Co. KG, Wiesbaden (DE)

(72) Inventor: Max DIETZ, Wiesbaden (DE)

(21) Appl. No.: 16/627,402

(22) PCT Filed: Jul. 9, 2018
(86) PCT No.: PCT/EP2018/068584

§ 371 Date: Dec. 30, 2019

(30) Foreign Application Priority Data
Jul. 7, 2017 (DE) 10 2017 006 447.4

(51) Int. Cl. A61L 31/10; A61L 31/16; A61L 27/58; A61L 31/14

(52) U.S. Cl. CPC A61L 31/10; A61L 31/16; A61L 2300/606; A61L 31/148; A61L 2300/416; A61L 27/58; A61L 2420/02

(57) ABSTRACT

The present invention relates to a biodegradable surface coating for inhibiting/delaying erosion/corrosion or degradation of solid materials for medical applications and their integration into cell assemblies.

The present invention relates to a biodegradable surface coating for inhibiting/delaying erosion/corrosion or degradation of solid materials for medical applications and their integration into cell assemblies.

### STATE OF THE ART

Fabrics and molding parts made of inorganic or organic compounds are very often used as medical devices or medical implants in the form of a support and holding device. Due to the good mechanical properties mainly metallic alloys are used for this purpose. However, metallic alloys have the disadvantage that when they come into contact with an aqueous medium, metal ions or metal particles can be liberated. Furthermore, there is an electrical surface potential that can lead to interference with living cells. Thus, it is known that the detachment of particles from stents leads to an increased inflammatory reaction of the vessel wall, which leads to an increase in the rate of re-narrowing of the treated vessel areas (Köster R, Vieluf D, Kiehn M, Sommerauer M, Kähler J, Baldus S, Meinertz T. Nick molybdenum contact allergies in patients with coronary in-stent restenosis, Lancet 2000, 356: 1895-1897). There are also reports of possible carcinogenicity of released metal particles (Kasprzak K S, Sunderman F W, Jr, Salnikov K. Nickel carcinogenesis, Mutat Res. 2003; 533: 67-97). Furthermore, an electrical surface potential can lead to activation of the coagulation system, which can lead, for example in the case of stents, to thrombotic occlusions which are life-threatening. Therefore, many methods have been developed to passivate and/or coat the corrosion of surfaces of metallic implant surfaces.

Surface coatings with hard inflexible coating materials, such as bioceramics (TiN, TiCN, Al₂O₃, SiC, BN or SiN₄), have been shown to be unsuitable when used on implants that are expanded and thereby deformed during their insertion, as is the case with stents since fracturing of the coating can occur (Sella C, Martin J C, Lecoeur, Le Chanu A, Harmand M F, Naji A, Davidas J P. Biocompatibility and corrosion resistance in biological media of hard ceramic coatings sputter deposited on metal implants. Mater Sci Eng A. 1991; 139:49-57). Therefore, implants are coated with resorbable or non-resorbable polymers. The disadvantage here is that all the polymers presented hitherto can enhance an inflammatory process by their presence and/or the degradation of polymer structures (Effect of stent coating alone on in vitro vascular smooth muscle cell proliferation and apoptosis. Antonio Curcio, Daniele Torella, Giovanni Cuda, Carmela Coppola, Maria Concetta Faniello, Francesco Achille, Viviana G. Russo, Massimo Chiariello, Ciro Indolfi. American Journal of Physiology—Heart and Circulatory Physiology Published 1 Mar. 2004 Vol. 286). Thus, for example, in the case of a polymer coating of a stent, it is necessary to incorporate an antiproliferative compound into the coating to prevent an otherwise increased likelihood of re-narrowing. On the other hand, the anti-proliferative substances used for this purpose, such as m-TOR inhibitors and paclitaxel, are cytotoxic and may themselves cause unwanted tissue reactions, such as neo-artheriosclerosis. Another disadvantage of the known polymer coatings is that in order to maintain the full area of the coating and its integrity, the arrangement of the coating layers requires a certain minimum thickness of the total coating, which is between 5 and 150 μm. This can also lead to surface irregularities, which in turn can lead to an increased adhesion of platelets and increased proliferation of adherent cells. Furthermore, it has been shown that coating gaps remain with thin polymer coatings, so that coating thicknesses which are generally more than 10 μm must be selected. Together with the resulting surface roughness, this makes insertion (advanceability) of vascular implants more difficult.

Medical implants are also made of metal alloys or polymers that are partially or completely degraded at the site of implantation over time. It has been found that a delay of the degradation is advantageous in a large number of applications. This applies, for example, to magnesium-based stents in which, without inhibition of the degradation, a loss of support function occurs after just a few days due to fractures of individual eroded/corroded struts. Therefore, hydrophobic alloys as well as degradable polymer coatings have been proposed, thereby extending the time until instability of such a stent occurs. However, it has been shown that the achievable time delay of the degradation is not sufficient and such coated stents are very difficult to advance in a vessel. Furthermore, an increased frequency of thrombosis formation has been reported.

Therefore, there is a great need for a surface coating with improved corrosion protection and at the same time low layer thickness, which does not cause activation of inflammatory processes.

A surface coating of medical implants may be desirable if, for example, improved wound healing can be achieved or the biocompatibility can be improved or the implant can or must also be protected against degradation and corrosive changes. Furthermore, surface coatings are used to release drugs locally. According to the prior art, different coating systems for medical-technical materials/implants are used for these different tasks. There is no need for most coating applications to be durable and resistant to abrasion. Rather, it is desired that these coatings be partially or completely dissolved or degraded over a certain period of time. Organic compounds which have sufficient biocompatibility are almost exclusively used for this purpose. However, the local biocompatibility is often not given when a large amount of degraded/released compounds of the coating system accumulate, so that immunostimulatory effects result during degradation.

Corrosion of metals or metal alloys or degradation of organopolymers such as PLLA or PGLA is already initiated by brief contact with water. In particular, the simultaneous presence of electrolytes accelerates the corrosion processes. In principle, the same applies to many metal alloys. Therefore, it is advantageous to provide a water-impermeable barrier on the surface of a medical-technical material and in particular of implants, in order to reduce or prevent the corrosion or degradation of the implant. Water impermeable surface sealants for technical applications are known in the art. Such coatings, however, are not useful for medical instruments and implants since they are not biocompatible and/or require a thickness of the layered construction that can not be used for most implants. Hydrolytic corrosion of metals or corrosion of organopolymers releases compounds such as metal ions and metal oxides or lactic acid or even hydrogen which, when passing through a coating, lead to activation of cellular or plasmatic systems. This can be induced even due to the smallest amounts of degradation products. Such hydrophilic degradation products can penetrate layers containing water through diffusive processes driven by an electrical gradient or a diffusion gradient, thereby entering an adjacent water phase.

For biocompatible coatings of the prior art, it is not known that the water permeability is or can be reduced to a relevant extent. Rather, preferably hydrophilic compounds are used to ensure high biocompatibility and degradability of the coating substances. In particular, polymeric coatings are therefore predominantly hydrophilic and absorb water. Although water permeability can be reduced by increasing layer thicknesses and a high degree of crosslinking of polymers; however, such layer thicknesses of polymers are known to cause severe cellular reactions which is accomplished through monocytic degradation and the resulting degradation products of the polymers.

Thus, another problem is to prevent passage of water and degradation products through a surface coating without causing activation of coagulation factors or adherent cells and/or without having high long-term stability and/or being eliminated by a diffusive dissolution and dissolution process, i.e., not causing monocytic degradation, without being metabolized by adhering cells or eliminated via a lymphatic transport system.

In the prior art, no surface coating is known which on the one hand exhibits a sufficiently large barrier against penetration of water molecules (impermeable to water), allows high applicability/advanceability to/in the vascular systems, is flexible, and is also biocompatible and biodegradable.

Therefore, the object of the invention is to provide a surface coating which on the one hand is biocompatible, biodegradable and flexible, and on the other hand impermeable to water. In addition, another object is to provide a surface coating with which medical products can be provided with very thin layer thicknesses and is also biocompatible, biodegradable, flexible and impermeable to water. In addition, the object of such a coating is that it delays or prevents erosion/corrosion and/or degradation of the coated material.

This object is achieved by the technical teaching of the independent claims. Further advantageous embodiments of the invention will become apparent from the dependent claims, the description, the figures and the examples.

### DESCRIPTION

The present object is achieved by providing a surface coating comprising or consisting of at least one carboxylic acid; and at least one hydrophobic cationic electrolyte or hydrophobic cationic polyelectrolyte or a mixture comprising at least one hydrophobic cationic electrolyte and at least one hydrophobic cationic polyelectrolyte, wherein surface coating is made (prepared) under anhydrous conditions.

In other words, the present invention relates to the provision of a surface coating for medical devices, in particular medical devices in contact with blood, comprising or consisting of at least one carboxylic acid; and at least one hydrophobic cationic electrolyte or hydrophobic cationic polyelectrolyte or a mixture comprising at least one hydrophobic cationic electrolyte and at least one hydrophobic cationic polyelectrolyte, wherein surface coating is made (prepared) under anhydrous conditions.

One embodiment of the present invention relates to a surface coating comprising or consisting of at least one carboxylic acid; and at least one hydrophobic cationic electrolyte or hydrophobic cationic polyelectrolyte or a mixture comprising at least one hydrophobic cationic electrolyte and at least one hydrophobic cationic polyelectrolyte, wherein surface coating is made (prepared) under anhydrous conditions.

Another aspect of the present invention is a medical product coated with a surface coating according to the invention.

An embodiment of the invention is directed to a medical product having a surface coating comprising or consisting of at least one carboxylic acid; and at least one hydrophobic cationic electrolyte or hydrophobic cationic polyelectrolyte or a mixture comprising at least one hydrophobic cationic electrolyte and at least one hydrophobic cationic polyelectrolyte, wherein the surface coating is made (prepared) under anhydrous conditions

It has been shown that an effective delay or inhibition of erosion/corrosion or degradation processes of the coated materials is determined in particular by efficient isolation of the material against penetration of water molecules and/or ions. It was found that, especially in an aqueous medium with dissolved electrolytes, such an efficient barrier, which also requires electrical insulation and is characterized by the electrical surface resistance, is suitable for this purpose.

“Isolating” or “isolation” as used herein means that mass transfer in both directions is not possible for a given time interval. The term “insulating” or “isolation” includes reducing the penetration of water molecules and/or ions, which means reduction of the permeability of water molecules and/or ions or the complete impermeability of water molecules and/or ions. Furthermore, the term may include electrical isolation. For example, a reduction or complete elimination of water permeability may be recognized by a delay/inhibition of corrosion/corrosion of water-erodible or corrodible materials such as iron or magnesium. Therefore, the extent of the erosion/corrosion delay can be used to quantify the water impermeability. The reduction in water impermeability as referred to herein is at least >90%, more preferably >95%, and more preferably >98% compared to an uncoated surface when the material is immersed in an aqueous medium such as physiological solutions such as PBS or NaCl for the period of at least 4 weeks.

When metallic surfaces are inserted into an aqueous medium, a galvanic voltage or a current flow is created. “Electrical isolation” as used herein means the reduction or complete avoidance of galvanic voltage or current flow. A “reduction” is present if the galvanic voltage or a current flow is reduced by at least 95%, more preferably 98%, calculated on the difference between the coated and uncoated metallic surface. The electrical insulation can be characterized by the surface electrical resistance.

From the degradation reduction, which affects both the coating itself according to the invention, as well as erosion and corrosion processes of the coated material, there are further advantageous effects. The contact of cells with a foreign material causes an immunological reaction that provokes a proinflammatory effect. In the case of such contact activation in a region in which an immunological reaction has already been triggered, e.g. by a tissue trauma, there is a synergistic amplification of the immunological reactions.

This applies, for example, to the implantation of vascular supports in the treatment of coronary artery lesions or for the implantation of a surgical mesh in the treatment of an infected hernia. There may be other factors that have proinflammatory effects. For example, a pH shift or a high ion concentration or a shift in the osmolality of the tissue fluid surrounding the implant cause a potentiation of the immunological reaction. In the corrosion and degradation of implant materials, compounds are released that cause one or more of the aforementioned local effects, e.g. by release of hydrogen ions or lactic acid. Such an immunological reaction does not occur, or only to a minor extent, if there is no inflammatory reaction in the tissue section in which the foreign material decomposes. It has already been shown that cells which come into contact with a foreign surface that has been coated with nitro-fatty acids exhibit better cell homing than in the case of the native foreign surface. This can be evidenced, i.e. cells that adhere more frequently and more strongly and at the same time do not increase proliferation, which is not the case in the native foreign surface; furthermore there is only a single-cell cell layering and not formation of a cellular multi-layering. A disadvantage of a surface coating with nitro-fatty acids is that usually only a monolayer of those compounds can be applied. Although the electrostatically bound nitro-fatty acids, due to their strong hydrophobicity, exhibit a significantly lower detachment behavior of surfaces when stored in an aqueous medium compared to the corresponding non-nitrated fatty acids, there is no long-term stability in an aqueous medium. Therefore this can lead, in particular in the case of degradable materials in which coating with nitro-fatty acids does not persist sufficiently long, to proinflammatory processes that occur as a result of an already occurring degradation, which nevertheless can lead to undesired tissue reactions. Therefore, it is also an object of the invention to inhibit both the erosion/corrosion processes of the coated material as long as possible and at the same time to ensure/provide a superficial coverage with nitro-fatty acids which remains stable until completion of immunological reactions in the tissue section of the implantation or contacting, or in the event of premature detachment, complete surface coverage of nitro-fatty acids is retained by subsequent replacement of nitro-fatty acids that diffuse out of the layered construction to the surface (and therefore is self-regenerating/healing). It has been shown that this is possible by using a surface coating according to the invention.

It could be documented that the proinflammatory reactions which occur, for example, due to the hydrolytically produced acid groups in the degradation of polylactide polymers are completely absent for a sufficiently long period by coating such a material surface. This is due in particular to the complete prevention of degradation of the implant material during this time. Thus, for the first time, a biocompatible and biodegradable surface coating for medical or medical grade solid materials can be provided to reduce or inhibit the immunostimulatory/proinflammatory and/or proliferative stimulus of the material surface of the solid material that reaches beyond the period of time that is needed for physiologic healing/healing of concomitant/underlying injury/trauma of/to the tissue adjacent to the solid material. Thus, an immunological reaction of the tissue or bodily fluid adjacent to a medical or medical material coated according to the present invention which exceeds a physiological response can be prevented.

Another embodiment of the present invention is a surface coating for corrosion and/or degradation delay, comprising or consisting of at least one carboxylic acid; and at least one hydrophobic cationic electrolyte or hydrophobic cationic polyelectrolyte or a mixture comprising at least one hydrophobic cationic electrolyte and at least one hydrophobic cationic polyelectrolyte, wherein the surface coating is made under anhydrous conditions.

In other words: a hydrophobic, biodegradable and insulating surface coating for retardation of corrosion and/or degradation of solid materials, comprising or consisting of at least one carboxylic acid; and at least one hydrophobic cationic electrolyte or hydrophobic cationic polyelectrolyte or a mixture comprising at least one hydrophobic cationic electrolyte and at least one hydrophobic cationic polyelectrolyte, wherein the surface coating is made under anhydrous conditions. Preferably, the surface coating is biodegradable and insulating and/or self-regenerating.

The surface coatings according to the invention preferably have a water contact angle of ≥80° or >80°, more preferably ≥90° or >90°, more preferably ≥100° or >100°, more preferably ≥110° or >110°, further preferably ≥120° or >120°, more preferably ≥130° or >130°, more preferably ≥140° or >140°, even more preferably ≥150° or >150°, ≥160° or >160° and most preferably ≥170° or >170°. Water contact angle is usually measured at a standard pressure of 1.013 bar and standard temperature of 25° C.

Another aspect of the present invention is an anhydrous composition for corrosion and/or degradation retardation comprising or consisting of at least one carboxylic acid; and at least one hydrophobic cationic electrolyte or hydrophobic cationic polyelectrolyte or a mixture comprising at least one hydrophobic cationic electrolyte and at least one hydrophobic cationic polyelectrolyte for use in medicine in the form of a coating.

Furthermore, another aspect of the present invention is an anhydrous corrosion and/or degradation retardant composition comprising or consisting of at least one carboxylic acid; and at least one hydrophobic cationic electrolyte or hydrophobic cationic polyelectrolyte or a mixture comprising at least one hydrophobic cationic electrolyte and at least one hydrophobic cationic polyelectrolyte for use in the treatment of arteries, in particular arterial stenoses, in the form of a coating.

A measure of the permeability of a barrier layer by water molecules is, among others, the ion conductivity/electron conductivity, or the electrical resistance of such a coating, which is also referred to below as insulation resistance or surface resistance. Therefore, the completeness of the insulation achieved by a coating was determined by measuring the ionic conductivity of a coated metallic substrate in an electrolyte bath (0.9% NaCl and 0.5% Na2SO4). An indirect indication of penetration of water molecules through a barrier, or corrosion/erosion, may be leakage of ions or degradation products (e.g., hydrogen ions) from the coated material into an aqueous medium in which the coated materials are immersed in order to verify long-term stability. For example, the change in the electrical conductivity of the aqueous medium in which the substrates are immersed can be used as an overall parameter to measure electrical conductivity.

It is known from the prior art that polyelectrolytes are suitable for surface coating because they are very easily applied in dissolved form in an aqueous medium to surfaces with opposite charge, thus, ultimately resulting in a surface with a high resistance when brought into contact therewith.

However, the aqueous polyelectrolyte systems known from the prior art are of low or medium viscosity, so that they can nevertheless be detached from surfaces in an aqueous medium by shear flow. Therefore, according to the prior art, multiple layers of coating are applied with polyelectrolytes which are applied in alternating sequence with opposite signs in their charged groups. Such coatings can not be prepared in an anhydrous manner, so they do not prevent degradation of the substrate they cover. This is mainly due to the fact that the substances previously used for the coating are highly water soluble and at the same time are not soluble in organic solvents, at least not alone. Furthermore, the said substances already contain water because of their high water affinity. Furthermore, the said substances are viscous in pure form and therefore unsuitable for the application of thin layers.

Another problem is that biocompatible compounds known for surface coating of the prior art are applied in the form of aqueous solutions. When using coating substances that were dissolved in an aqueous system, it has been shown that exposure to water even for a short period is sufficient to initiate superficial degradation in degradable materials/implants. It could be shown that after initiation of a water-mediated degradation/erosion, which among others leads to the formation of free ions or hydroxides, even after drying and obtaining of an anhydrous surface, and even after application of a largely water-impermeable layer, corrosion/degradation occurs significantly earlier and faster in these areas, as is the case with coated surfaces where no such surface activation by water contact has been taken place. Thus, especially in the presence of ions, which are located at the interface between the material and the coating, osmotic effects occur that can hardly be prevented by a thin barrier layer and which lead to hydration of the boundary layer (below the coating). At sufficient vapor pressure, water molecules can also penetrate a two-dimensional composite which, for example, consists of polymers that are covalently bound to one another.

Therefore, coating systems in which the surfaces to be coated come into contact with water and/or the coating compounds have a water sheath, in particular for coating degradable implants, are not suitable.

Therefore, no surface coatings for medical-technical materials are known in the prior art that on the one hand ensure a sufficiently large protection against penetration of water molecules or ions and on the other can be applied in the form of thin and flexible layers which are in the range of <100 μm, and which are biocompatible and biodegradable.

It is known from the prior art that nitro group-bearing carboxylic acids can be used for the surface coating of medical materials/implants. For surface coatings with nitro-group-bearing fatty acids (nitro-fatty acids) it has also been found that this can lead to a passivation of attached cells. It is also known that fatty acids can be deposited from an organic solvent phase onto surfaces. Advantageously, very thin layers can be obtained, e.g. in the form of monolayers. In this case, closed layers which have marked surface hydrophobicity exhibiting a water contact angle of 60° to 90° can be produced. However, it has been shown that single- or multi-layer coatings with fatty acids are not sufficient to prevent the passage of water molecules through such a coating with fatty acids. It could also be shown that no electrical insulation can be achieved herewith. Diffusion of water molecules through a monolayer of non-nitrated and nitro-fatty acids leads to hydration of the carboxyl groups of the fatty acid molecules. It is known that electrostatic attachment of the carboxyl group of carboxylic acids onto a metal surface leads to acid-catalytic acceleration of corrosion/corrosion processes due to a hydration of the carboxyl groups. This is the case in particular with degradable materials, such as, for example, magnesium alloys or lactic acid derivatives.

In the prior art, biodegradable endoprostheses are known which should remain in the body until the area in the body that has been treated has healed. However, the previously known biodegradable endoprostheses decompose under physiological conditions before the healing of the treated area in the body has completed. Therefore, in particular endoprostheses consisting of a degradable alloy or a polymer with a carboxylic acid coating can not meet the said requirements.

Surprisingly, it has been found that ultra-thin, water-impermeable surface coatings which are applied to them without water and without erosion/degradation of the surfaces can be produced and these have very good biocompatibility and are metabolized completely and without cell activation.

It has been found that a layered construction of a hydrophobic cationic polyelectrolyte and a hydrophobic carboxylic acid, which is produced under the application conditions according to the invention, enables these advantageous effects. Furthermore, the layered structure brings this allows very advantageous biological effects.

The surface coating according to the invention is therefore preferably biodegradable. A preferred embodiment of the present invention is therefore directed to a surface coating comprising at least one carboxylic acid and at least one hydrophobic cationic electrolyte or hydrophobic cationic polyelectrolyte or a mixture comprising at least one hydrophobic cationic electrolyte and at least one hydrophobic cationic polyelectrolyte, wherein the surface coating is prepared in an anhydrous manner and is bio-resorbable.

Therefore, another aspect of the present invention is directed to a method of making surface coatings comprising the following steps:

a) providing a solid material,

b) wetting of the surface of the solid material under anhydrous conditions with at least one hydrophobic cationic electrolyte or at least one hydrophobic cationic polyelectrolyte or a mixture comprising at least one hydrophobic cationic electrolyte and at least one hydrophobic cationic polyelectrolyte,

c) drying the surface,

d) wetting of the surface under anhydrous conditions with at least one carboxylic aci,

e) rinsing and drying the surface, and

f) obtaining of the surface coating.

Preference is given to a method for suppressing the release of proinflammatory and proliferation-promoting erosion/corrosion products from medical implants.

Preferred is a method of providing reservoir formation in a coating composition with carboxylic acids/nitro-carboxylic acids which enable cell homing to surfaces of medical implants and wound care materials coated therewith.

Preferred is a method in which a degradation delay of a medical device that is surrounded by a bodily fluid or at least in an area adjacent hereto and/or a in which degradation delay of the coating with carboxylic acids/nitro-carboxylic acids reduces or prevents proliferation of fibroblasts, endothelial cells, epithelial cells or leukocytes.

A surface coating is preferred for reducing or avoiding restenosis and/or thrombosis after implantation of a vascular implant.

A preferred embodiment is directed to a surface coating according to the invention, wherein the surface coating comprises a carboxylic acid layer comprising the at least one carboxylic acid and an electrolyte layer comprising the at least one hydrophobic cationic electrolyte or hydrophobic cationic polyelectrolyte or a mixture comprising the at least one hydrophobic cationic electrolyte and/or the at least one hydrophobic cationic polyelectrolyte, wherein the surface coating is made under anhydrous conditions.

A further preferred embodiment is directed to a surface coating according to the invention, the surface coating comprising a carboxylic acid layer comprising the at least one carboxylic acid and an electrolyte layer comprising the at least one hydrophobic cationic electrolyte or hydrophobic cationic polyelectrolyte or a mixture comprising the at least one hydrophobic cationic electrolyte and/or the at least one hydrophobic cationic polyelectrolyte, wherein the surface coating is made under anhydrous conditions, wherein the carboxylic acid layer is on the electrolyte layer.

In a method according to the invention in step b), the surface of the solid material from step a) is preferably brought into contact under anhydrous conditions with at least one hydrophobic cationic electrolyte or at least one hydrophobic cationic polyelectrolyte or a mixture comprising at least one hydrophobic cationic electrolyte and at least one hydrophobic cationic polyelectrolyte. A process according to the invention with a step c) drying of the surface after step b) is preferred. Preference is furthermore given to a process according to the invention comprising a step d) wetting under anhydrous conditions of the surface from step c) with at least one carboxylic acid. Furthermore, a method according to the invention preferably contains step e) rinsing and drying of the surface from step d).

In order to remove solvents, they are dried in step c). In particular, it is meant that an organic solvent is completely removed from the coating. Drying may be performed at elevated or reduced temperatures.

Therefore, another aspect of the present invention is directed to a method of making surface coatings, comprising the following steps:

a) providing a solid material,

b) wetting of the surface of the solid material from step a) under anhydrous conditions with at least one hydrophobic cationic electrolyte or at least one hydrophobic cationic polyelectrolyte or a mixture comprising at least one hydrophobic cationic electrolyte and at least one hydrophobic cationic polyelectrolyte,

c) drying the surface after step b),

d) wetting of the surface from step c) under anhydrous conditions with at least one carboxylic acid,

e) rinsing and drying the surface of step d),

f) obtaining the surface coating.

A preferred embodiment of the present invention relates to a process for producing a surface coating, comprising the following steps:

a) providing a solid material with a cleaned and/or hydrophobized material surface,

b) wetting of the surface of the solid material from step a) under anhydrous conditions with at least one hydrophobic cationic electrolyte or at least one hydrophobic cationic polyelectrolyte or a mixture comprising at least one hydrophobic cationic electrolyte and at least one hydrophobic cationic polyelectrolyte,

c) drying the surface after step b),

d) wetting of the surface from step c) under anhydrous conditions with at least one carboxylic acid,

e) rinsing and drying the surface of step d), and

f) obtaining the surface coating.

A preferred embodiment of the present invention relates to a process for producing a surface coating comprising the following steps:

a) providing a solid material with material surface that has been cleaned and/or rendered hydrophobic,

b) wetting of the surface of the solid material under anhydrous conditions with at least one hydrophobic cationic electrolyte or at least one hydrophobic cationic polyelectrolyte or a mixture comprising at least one hydrophobic cationic electrolyte and at least one hydrophobic cationic polyelectrolyte,

c) drying the surface,

d) wetting of the surface under anhydrous conditions with at least one carboxylic acid,

e) rinsing and drying the surface, and

f) obtaining the surface properties.

The processes according to the invention are preferably directed to processes for producing a hydrophobic, biodegradable, biocompatible and insulating surface coating, in particular preferably to corrosion and/or degradation delay of solid materials. It is therefore preferred if a biodegradable, biocompatible surface coating with hydrophobic surface properties is obtained in step f). Another aspect of the present invention is directed to a process for producing a hydrophobic, biodegradable and insulating surface coating to retard corrosion and/or degradation of solid materials.

Therefore, another aspect of the present invention is directed to a process for producing surface coating for producing a hydrophobic, biodegradable and insulating surface coating for corrosion and/or degradation delay of solid materials comprising the following steps:

a) providing a solid material,

b) wetting of the surface of the solid material under anhydrous conditions with at least one hydrophobic cationic electrolyte or at least one hydrophobic cationic polyelectrolyte or a mixture comprising at least one hydrophobic cationic electrolyte and at least one hydrophobic cationic polyelectrolyte,

c) drying the surface,

d) wetting of the surface under anhydrous conditions with at least one carboxylic acid,

e) rinsing and drying the surface, and

f) obtaining the surface coating.

A layer comprising at least one hydrophobic cationic electrolyte or at least one hydrophobic cationic polyelectrolyte or a mixture comprising at least one hydrophobic cationic electrolyte and at least one hydrophobic cationic polyelectrolyte is also referred to below as the “electrolyte layer”. “Polyelectrolyte layer” as used herein refers to a layer comprising at least one hydrophobic cationic polyelectrolyte or a mixture comprising at least one hydrophobic cationic polyelectrolyte.

Another aspect of the present invention is directed to a surface coating obtainable or obtained by a process according to the invention. A hydrophobic, biocompatible, biodegradable and/or insulating surface coating is preferred.

One embodiment of the present invention is directed to a surface coating obtainable or obtained by a process for producing a hydrophobic, biodegradable and insulating surface coating for retardation of corrosion and/or degradation of solid materials comprising the following steps:

a) providing a solid material,

b) wetting of the surface of the solid material under anhydrous conditions with at least one hydrophobic cationic electrolyte or at least one hydrophobic cationic polyelectrolyte or a mixture comprising at least one hydrophobic cationic electrolyte and at least one hydrophobic cationic polyelectrolyte,

c) drying the surface,

d) wetting of the surface under anhydrous conditions with at least one carboxylic acid,

e) rinsing and drying the surface, and

f) obtaining the surface coating.

In step a), a solid material comprising a material surface that has been cleaned and/or rendered hydrophobic is preferably provided.

Another aspect of the present invention is a medical product having a surface coating obtainable or obtained by a method of the invention. It is preferably a hydrophobic, biodegradable, biocompatible and insulating surface coating.

Hydrophilic cationic polyelectrolytes are widely used in the prior art, for example as flocculants in water treatment or as stabilizers in suspensions of aqueous media. On the other hand, hydrophobic cationic electrolytes are less common.

They are used for example in building materials, such as cement. Positively charged groups are provided predominantly by quaternized nitrogen compounds. But also other charged carriers are suitable, such as imines, azanes, triazanes, tetrazanes, nitrones. DE10124387A1 discloses methods with which hydrophilic cationic polyelectrolytes can be hydrophobically functionalized.

As described below, a prerequisite for achieving an anti-erosion/corrosion-resistant coating and insulation of the coated material according to the coating methods of the present invention is that a cationic electrolyte or polyelectrolyte deposited on a material surface from an anhydrous phase and binds with high adhesion force. Since a covalent binding/compounding of the compounds to be used for surface coating is not desired in order to allow good biodegradability and to ensure the lowest possible requirement needed for activation of cellular degradation processes, it is also the object of the invention to provide an insulating layered structure, which ensures stability just by hydrophobic binding forces.

It has now been found that hydrophobic cationic polyelectrolytes can be applied under anhydrous conditions to the surfaces of different materials to form a closed film. The preferably physiosorptively applied hydrophobic cationic polyelectrolytes have high binding stability and virtually do not dissolve in an aqueous medium. However, a multi-layered construction of hydrophobic cationic polyelectrolytes also failed to achieve electrical isolation of the coated material so that water molecules could pass through a coating of hydrophobic cationic polyelectrolytes and result in erosion/corrosion of the coated material. Therefore, hydrophobic cationic polyelectrolytes having different ratios of charged-group carrying linkage moieties and carbon chain lengths were investigated. It was found that as the number of charged groups decreased and/or the carbon chain lengths increased, as well as the degree of branching, although hydrophobicity increased, isolation of a surface coated therewith could not be ensured.

Surprisingly, an electrical insulation could be achieved by applying a self-assembling layer of fatty acids from an anhydrous solution to an electrolyte layer that was prepared under anhydrous conditions.

It has also been found that electrical insulation is not achieved when the coating was applied with the same or similar compounds, but using a solution that contained any water.

This applies both to the application of the electrolyte layer (hydrophobic cationic electrolyte and/or hydrophobic cationic polyelectrolytes) and to the application of the carboxylic acids. Preferred is a process according to the invention described herein for producing a biodegradable surface coating for inhibiting/delaying erosion/corrosion or degradation of solid materials comprising a single- or multi-layered structure comprising at least one hydrophobic cationic electrolyte or hydrophobic cationic polyelectrolyte or a mixture comprising at least one hydrophobic cationic electrolyte and at least one hydrophobic cationic polyelectrolyte and a carboxylic acid, carried out by means of an application in an anhydrous manner. In a single-layered construction, the surface layer preferably consists of a layer comprising at least one hydrophobic cationic electrolyte or at least one hydrophobic cationic polyelectrolyte or a mixture comprising at least one hydrophobic cationic electrolyte and at least one hydrophobic cationic polyelectrolyte and also at least one carboxylic acid.

Therefore, a preferred embodiment of the present invention is a process for producing a hydrophobic, biodegradable and insulating surface coating for corrosion and/or degradation retardation of solid materials comprising the following steps:

a) providing a solid material,

b) wetting of the surface of the solid material under anhydrous conditions with at least one hydrophobic cationic electrolyte or at least one hydrophobic cationic polyelectrolyte or a mixture comprising at least one hydrophobic cationic electrolyte and at least one hydrophobic cationic polyelectrolyte,

c) drying the surface,

d) wetting of the surface under anhydrous conditions with at least one carboxylic acid,

e) rinsing and drying the surface,

f) obtaining the surface coating, and

wherein a single- or multi-layered construction consists of at least one hydrophobic cationic electrolyte or hydrophobic cationic polyelectrolyte or a mixture comprising at least one hydrophobic cationic electrolyte and at least one hydrophobic cationic polyelectrolyte and the at least one carboxylic acid, and wherein the layer application takes place in each case in an anhydrous manner.

Furthermore, a surface coating for inhibiting/delaying erosion/corrosion or degradation of solid materials is obtainable or obtained by a method of the present invention described herein, wherein a single or multi-layered construction of at least one hydrophobic cationic electrolyte or hydrophobic cationic polyelectrolyte or a mixture comprising at least one hydrophobic cationic electrolyte and at least one hydrophobic cationic polyelectrolyte and a carboxylic acid is accomplished by means of an application in an anhydrous manner.

Preference is furthermore given to a surface coating according to the invention for corrosion and/or degradation retardation of solid materials described herein, wherein a single-layer or multi-layer layer structure of a hydrophobic cationic polyelectrolyte and a carboxylic acid is effected by means of an application in an anhydrous manner.

In a preferred embodiment of the present invention, the surface coating comprises or consists of at least one carboxylic acid for the corrosion and/or degradation delay of solid materials; and at least one hydrophobic cationic electrolyte or hydrophobic cationic polyelectrolyte or a mixture comprising at least one hydrophobic cationic electrolyte and at least one hydrophobic cationic polyelectrolyte, wherein a single- or multi-layered construction consists of a layer comprising or consisting of the at least one hydrophobic cationic electrolyte or hydrophobic cationic polyelectrolyte or a mixture comprising at least one hydrophobic cationic electrolyte and at least one hydrophobic cationic polyelectrolyte and a layer comprising or consisting of the at least one carboxylic acid, and wherein the layer coating is carried out in each case under anhydrous conditions.

It has been found that it is possible to coat the anhydrous electrolyte layer with anionic compounds that were present in dissociated form in an aqueous medium, but because water enters the layered construction, electrical insulation of the coating does not result. In addition, adhered compounds were easily removed again by an aqueous flushing medium.

Liquid carboxylic acids having different chain lengths could be applied directly to hydrophobic cationic polyelectrolytes under anhydrous conditions by known methods, such as the micropipetting method or a spray coating, which also results in a surface hydrophobicity with water contact angles of ≥80° or >80°, more preferably ≥90° or >90°, more preferably ≥100° or >100°, more preferably ≥110° or >110°, more preferably ≥120° or >120°, more preferably ≥130° or >130°, further preferably ≥140° or >140°, even more preferably ≥150° or >150°, ≥160° or >160° and most preferably ≥170° or >170°.

Carboxylic acid compounds deposited in this manner could be removed again in part by rinsing with an ethanol solution.

Surprisingly, a high surface coverage (per unit area) and surface stability of carboxylic acids could be achieved by depositing fatty acids having a chain length of >5 when applied onto anhydrously produced hydrophobic cationic polyelectrolyte layers prepared under anhydrous conditions by using an anhydrous surface coating method using organic solvents.

Coverage stability is the time-dependent release of carboxylic acids when placed in an aqueous medium at 37° C. Those skilled in the art will appreciate that coverage stability is considered relative to other coatings.

In one embodiment of the present invention, the process for producing a hydrophobic, biodegradable and insulating surface coating for retardation of corrosion and/or degradation of solid materials comprises the following steps:

a) providing a solid material,

b) wetting the surface of the solid material under anhydrous conditions with at least one hydrophobic cationic electrolyte or at least one hydrophobic cationic polyelectrolyte or a mixture comprising at least one hydrophobic cationic electrolyte and at least one hydrophobic cationic polyelectrolyte, using an organic solvent,

c) drying the surface,

d) wetting of the surface under anhydrous conditions with at least one carboxylic acid, using an organic solvent,

e) rinsing and drying the surface,

f) obtaining the surface coating.

The completeness of the coverage was determined by an increase in the surface hydrophobicity, which was determined e.g. by water contact angle measurements and the electrical resistance of the surface coating. However, the measurable surface hydrophobicity did not increase further with chain lengths of the saturated and unbranched carboxylic acids greater than 14, at the same time the achievable surface resistance decreased with increasing chain length and degree of branching as well as the proportion of unsaturated carboxylic acids with unsaturated double bonds in cis configuration.

Surprisingly, a significantly greater electrical surface resistance of such a water-free surface coating was achieved if a nitro-fatty acid or a mixture of non-nitrated carboxylic acids with an aqueous nitro-fatty acid was applied under anhydrous conditions after application of a hydrophobic cationic polyelectrolyte in an anhydrous manner. No or no relevant amounts of nitrated and/or non-nitrated fatty acids could be removed from surfaces coated in this manner by aqueous or alcoholic solutions, recognizable by the unchanged or nearly unchanged (+/−10%) water contact angels before or after a rinsing and unmodified or almost unchanged (+/−10%) electrical resistance of these surfaces. Surface coatings prepared according to the invention have water contact angles of ≥80° or >80°, more preferably ≥90° or >90°, more preferably ≥100° or >100°, more preferably ≥110° or >110°, more preferably ≥120° or >120°, more preferably ≥130° or >130°, more preferably ≥140° or >140°, even more preferably ≥150° or >150°, ≥160° or >160° and most preferably ≥170° or >170°.

It has been found that a multi-layered construction, which is carried out by repeating steps b) and c) before step d), increases the degradation stability. Furthermore, the number of defects leading to electrical conductivity is also reduced by multiple coatings. Multiple coatings increase the manufacturing cost and leads to a higher overall thickness of the layered construction, which is not desirable for certain materials and is not required, since sufficient stabilization of the applied fatty acids, or nitro-fatty acids, against premature detachment is already ensured by the application of a single layer. On the other hand, it must be ensured that a complete coverage takes place, so that, depending on the material surface, it may be advantageous to perform multiple coatings. Therefore, in an application in which delayed degradation of a surface coverage with carboxylic acids and in particular with nitro-fatty acids is in the forefront, it is preferable to perform a 3-layered construction, more preferably a 2-layered construction and more preferably a 1-layer construction. If the most complete and long-lasting degradation delay and electrical insulation are in the forefront of the application, it may be advantageous to ensure the thickest possible layered construction, which is achieved by the application of multiple layers. Therefore, in such an application, it is preferable to form a 4-layer, more preferably a 6-layer, more preferably an 8-layer, and even more preferably a 10-layer construction. However, the number of layers is not limited, so even more layers can be applied.

Thus, an improvement/extension of the degradation stability is achieved by a coating according to the invention in many respects: on the one hand, the attachment of carboxylic acids and in particular nitro-fatty acids on the surface of the coating is significantly increased, and thus the time to a detachment/degradation, which leads to a change in physico-chemical (loss of hydrophobicity) and biological properties is significantly prolonged as compared with a coverage of the carboxylic acid on a native material surface. On the other hand, the coating accomplishes a significant delay in exposing the native material surface to an aqueous medium or biological system/organism. Consequently, this also significantly delays potential erosion/corrosion of the material surface. However, the biological reaction to the material surface and/or the erosion/corrosion product is also delayed. Furthermore, in the case of resorbable materials, the degradation processes, which are usually initiated or maintained by contact with water, are delayed and the function of the coated material, which as a rule has mechanical tasks, is maintained over a relatively long period of time. Therefore, improved degradation stability is desired and usable in various fields.

Preferred is a process for producing a biodegradable surface coating for inhibiting/delaying erosion/corrosion or degradation of solid materials, wherein a single or multi-layered layered construction of a hydrophobic cationic polyelectrolyte and a carboxylic acid preferably comprises at least one nitro-fatty acid or a mixture comprising or consisting of at least nitro-fatty acids and at least one non-nitrated fatty acids is applied by means of an anhydrous application method.

In the inventive methods of preparing the surface coating described herein and the surface coatings of the invention described herein, the at least one carboxylic acid is preferably a fatty acid, more preferably a nitrated carboxylic acid, and most preferably a nitrated carboxylic acid wherein the carboxylic acids have a carbon chain length of 6 to 24, more preferred from 8 to 22, more preferably from 10 to 20, more preferably from 12 to 20, and most preferably from 14 to 18.

In a preferred embodiment of the present invention, the method for producing a hydrophobic, biodegradable and insulating surface coating for retardation (delay) of corrosion and/or degradation of solid materials comprises the following steps:

a) providing a solid material,

b) wetting of the surface of the solid material under anhydrous conditions with at least one hydrophobic cationic electrolyte or at least one hydrophobic cationic polyelectrolyte or a mixture comprising at least one hydrophobic cationic electrolyte and at least one hydrophobic cationic polyelectrolyte,

c) drying the surface,

d) wetting of the surface under anhydrous conditions with at least one fatty acid having a carbon chain length of 6 to 24,

e) rinsing and drying the surface,

f) obtaining the surface coating.

In a preferred embodiment of the present invention, the process for producing a hydrophobic, biodegradable and insulating surface coating for corrosion and/or degradation delay of solid materials comprises the following steps:

a) providing a solid material,

b) wetting of the surface of the solid material under anhydrous conditions with at least one hydrophobic cationic electrolyte or at least one hydrophobic cationic polyelectrolyte or a mixture comprising at least one hydrophobic cationic electrolyte and at least one hydrophobic cationic polyelectrolyte,

c) drying the surface,

d) wetting of the surface under anhydrous conditions with at least one carboxylic acid, wherein the carboxylic acid is a nitro-fatty acid having a carbon chain length of 6 to 24,

e) rinsing and drying the surface,

f) obtaining the surface coating.

In a further preferred embodiment of the present invention, the method for producing a hydrophobic, biodegradable and insulating surface coating for the corrosion and/or degradation delay of solid materials comprises the following steps:

a) providing a solid material,

b) wetting of the surface of the solid material under anhydrous conditions with at least one hydrophobic cationic electrolyte or at least one hydrophobic cationic polyelectrolyte or a mixture comprising at least one hydrophobic cationic electrolyte and at least one hydrophobic cationic polyelectrolyte,

c) drying the surface,

d) wetting of the surface under anhydrous conditions with at least one carboxylic acid, wherein the carboxylic acids in step d) are mixtures of nitrated and non-nitrated fatty acids having a carbon chain length of 6 to 24,

e) rinsing and drying the surface,

f) obtaining the surface coating.

In a preferred embodiment of the present invention, the method for producing a hydrophobic, biodegradable and insulating surface coating for retardation of corrosion and/or degradation of solid materials comprises the following steps:

a) providing a solid material,

b) wetting of the surface of the solid material under anhydrous conditions with at least one hydrophobic cationic electrolyte or at least one hydrophobic cationic polyelectrolyte or a mixture comprising at least one hydrophobic cationic electrolyte and at least one hydrophobic cationic polyelectrolyte,

c) drying the surface,

d) wetting of the surface under anhydrous conditions with at least one carboxylic acid, wherein the carboxylic acid is a nitro-fatty acid or a mixture comprising or consisting of at least one nitro-fatty acid and at least one non-nitrated fatty acid, wherein the fatty acid has a carbon chain length of 6 to 24 having,

e) rinsing and drying the surface,

f) obtaining the surface coating.

It has been shown that the prevention/delaying of erosion/corrosion or degradation of solid materials according to the invention can be obtained by a simple and preferably monolayer coating with a hydrophobic cationic electrolyte layer and a fatty acid according to the invention, which have each been applied under anhydrous conditions. Such a layered construction has a layer thickness which is <50 μm, more preferably <10 μm, more preferably <1 μm, even more preferably <50 nm and most preferably <20 nm.

Although this can already achieve electrical insulation, a leakage of current may nevertheless occur when applying a high voltage. It has been found that a multilayered layered construction with a hydrophobic cationic electrolyte in combination with one of the fatty acids according to the invention further increases the insulation resistance. Therefore, a multi-layered layered construction with a hydrophobic cationic polyelectrolyte is a preferred method.

Preference is given to a method for producing a biodegradable surface coating for preventing/delaying an erosion/corrosion or degradation of solid materials, and to a surface coating according to the invention described herein, in which a single- or multi-layered layered construction consisting of a hydrophobic cationic polyelectrolyte and a final coating with or consisting of at least one non-nitrated fatty acid or at least one nitro-fatty acid or a mixture comprising or consisting of at least one nitro-fatty acid and at least one non-nitrated fatty acid whereby each layer is applied under anhydrous conditions. Particularly preferred is a final coating with at least one nitro-fatty acid or a mixture comprising or consisting of at least one nitro-fatty acid and at least one non-nitrated fatty acid which have been applied in an anhydrous manner. Most preferably, the final coating comprises or consists of at least one nitro-fatty acid which has been applied anhydrously.

The overall height of the finished surface coating having a layered construction according to the invention is preferably between 5 nm and 50 μm, more preferably between 10 nm and 45 μm, more preferably between 10 nm and 40 μm, 10 nm and 35 μm, more preferably between 10 nm and 30 μm, more preferably between 10 nm and 25 μm, more preferably between 15 nm and 20 μm, even more preferably between 15 nm and 15 μm and more preferably between 20 nm and 10 μm.

Preferred is a process according to the invention described herein for producing a hydrophobic, biodegradable and insulating surface coating for retarding corrosion and/or degradation of solid materials and the surface coating according to the invention described herein, wherein a single-layer or multi-layered layered construction of at least one hydrophobic cationic polyelectrolyte or a mixture is used, comprising at least one hydrophobic cationic electrolyte and at least one hydrophobic cationic polyelectrolyte and a final coating with nitro-fatty acid or a mixture of nitro-fatty acids and non-nitrated fatty acids by means of an anhydrous application and in which the total height of the layered construction is between 5 nm and 50 μm.

More preferably, a hydrophobic, biodegradable and insulating surface coating for delaying corrosion and/or degradation of solid materials is available or obtained by the method just described.

Cationic polyelectrolytes have extensive documentation of cytotoxic effects (Kafil V, Omidi Y. Cytotoxic Impacts of Linear and Branched Polyethyleneimines Nanostructures in A431 Cells, BioImpacts: BI, 2011; 1 (1): 23-30). The cytotoxicity increases, for example, with the degree of branching of polyethyleneimine. Surprisingly, in the degradation of the coating according to the invention, there were no signs of cytotoxic or immunological effects in cells adhering to such coated surfaces.

Although this could not be proven with certainty, it must be assumed that the highly delayed degradation achieved by the layered construction according to the invention and/or the in total only small amount of compounds to be metabolized and/or by uptake/incorporation of the inventively used compounds in the formed extracellular matrix or cellular structures do not lead to the cytotoxic effects that are known from hydrophilic cationic polyelectrolytes.

Preference is given to a method for producing a hydrophobic, biodegradable and insulating surface coating for the corrosion and/or degradation delay of solid materials and a surface coating according to the invention described herein, wherein a single or multi-layered layered construction of a hydrophobic cationic polyelectrolyte and a final coating with nitro-fatty acid or a mixture of nitro-fatty acids and non-nitrated fatty acids takes place by means of an application under anhydrous conditions, whereby cytotoxic effects do not occur during the degradation of the coating. Therefore, a surface coating according to the invention described herein is also obtained or obtainable by a process according to the invention described here which prevents or delays the erosion/corrosion or degradation of solid materials and is also suitable for preventing cytotoxic effects in case of degradation of the coating.

In inventively prepared surface coatings of samples consisting of corrosion-sensitive materials, such as iron or copper, no passage of hydrolysis/oxidation products could be detected following a coating according to the invention, while there was formation and passage of hydrolysis/oxidation products at/through coatings of such materials which have been made by means of aqueous solution systems in which hydrophilic cationic polyelectrolytes or carboxylic acids were dissolved.

No erosion/corrosion of a coated 316L steel alloy, a magnesium alloy and a copper alloy after 3 weeks storage time in an aqueous medium at 37° C. was detected, which is in accordance with a very low to no longer measurable conductivity of metallic surfaces which had been coated by a method according to the invention. For similar experiments with templates consisting of degradable bio-polymers, such as, a poly-L-lactic acid polymer or poly-glycolic acid polymer, no hydrolytic degradation products were detected in the aqueous storage medium. In all electron microscopic investigations which were carried out using templates that had been coated according to the invention, no substance defects of the surfaces due to a hydrolytic erosion/degradation were detected. In contrast, templates which had been stored in an aqueous medium over the same period of time and had no coating were completely or partially dissolved, and those which had been coated only with a hydrophobic cationic polyelectrolyte showed significant defects in the materials or had partially dissolved (magnesium alloy).

The surfaces coated according to the invention retained their hydrophobic surface properties which were unchanged over a period of at least 3 weeks during which they were placed in an aqueous solution at 37° C., recognizable by the fact that the water contact angle did not decrease more than 10° compared to the initial measurement. Preference is given to a method for producing a hydrophobic, biodegradable and insulating surface coating for the corrosion and/or degradation delay of solid materials or surface coating for corrosion and/or degradation of solid materials, in which a single or multilayer coating structure of at least one hydrophobic cationic electrolyte or a hydrophobic cationic polyelectrolyte or a mixture comprising at least one hydrophobic cationic electrolyte and at least one hydrophobic cationic polyelectrolyte and a final coating with nitro-fatty acid or a mixture of nitro-fatty acids and non-nitrated fatty acids is applied by means of an anhydrous application, whereby a water-resistant surface hydrophobicity has a water contact angle of ≥80° or >80°, more preferably ≥90° or >90°, more preferably ≥100° or >100°, more preferably ≥110° or >110°, further preferably ≥120° or >120°, more preferably ≥130° or >130°, more preferably ≥140° or >140°, even more preferably ≥150° or >150°, ≥160° or >160° and most preferably ≥170° or >170°, meaning that the surface coating has a hydrophobic water-resistant surface with a water contact angle of ≥80° or >80°, more preferably ≥90° or >90°, more preferably ≥100° or >100°, more preferably ≥110° or >110°, more preferably ≥120° or >120°, more preferably ≥130° or >130°, more preferably ≥140° or >140°, even more preferably ≥150° or >150°, ≥160° or >160° and most preferably ≥170° or >170°

A preferred embodiment of the underlying invention is directed to a hydrophobic, biodegradable and insulating surface coating for corrosion and/or degradation retardation of solid materials comprising the following steps:

a) providing a solid material,

b) wetting of the surface of the solid material under anhydrous conditions with at least one hydrophobic cationic electrolyte or at least one hydrophobic cationic polyelectrolyte or a mixture comprising at least one hydrophobic cationic electrolyte and at least one hydrophobic cationic polyelectrolyte,

c) drying the surface,

d) wetting of the surface under anhydrous conditions with at least one carboxylic acid,

e) rinsing and drying the surface,

f) obtaining the surface coating,

wherein a single- or multi-layered layered construction of at least one hydrophobic cationic electrolyte or hydrophobic cationic polyelectrolyte or a mixture comprising at least one hydrophobic cationic electrolyte and at least one hydrophobic cationic polyelectrolyte and a final coating with nitro-fatty acid or a mixture of nitro-fatty acids and non-nitrated fatty acids is carried out under anhydrous conditions, and whereby the hydrophobic surface has a water contact angle of ≥80° or >80°, more preferably ≥90° or >90°, more preferably ≥100° or >100°, more preferably ≥110° or >110°, more preferably ≥120° or >120°, more preferably ≥130° or >130°, more preferably ≥140° or >140°, even more preferably ≥150° or >150°, ≥160° or >160° and most preferably ≥170° or >170°, that means the surface coating has a hydrophobic water-resistant surface with a water contact angle of ≥80° or >80°, more preferably ≥90° or >90°, more preferably ≥100° or >100°, more preferably ≥110° or >110°, more preferably ≥120° or >120°, more preferably ≥130° or >130°, more preferably ≥140° or >140°, even more preferably ≥150° or >150°, ≥160° or >160° and most preferably ≥170° or >170°.

A preferred embodiment of the present invention is a surface coating for corrosion and/or degradation delay of solid materials comprising or consisting of at least one carboxylic acid; and at least one hydrophobic cationic electrolyte or hydrophobic cationic polyelectrolyte or a mixture comprising at least one hydrophobic cationic electrolyte and at least one hydrophobic cationic polyelectrolyte, wherein a monolayer or multilayered layered construction of at least one hydrophobic cationic electrolyte or hydrophobic cationic polyelectrolyte or a mixture comprising at least one hydrophobic cationic electrolyte and at least one hydrophobic cationic polyelectrolyte and a final coating with a nitro-fatty acid or a mixture of nitro-fatty acids and non-nitrated fatty acids by means of an anhydrous application, wherein the surface coating is water-resistant and has a water contact angle of ≥80° or >80°, more preferably ≥90° or >90°, more preferably ≥100° or >100°, more preferably ≥110° or >110°, further preferably ≥120° or >120°, more preferably ≥130° or >130°, more preferably ≥140° or >140°, even more preferably ≥150° or >150°, ≥160° or >160° and most preferably ≥170° or >170°.

In further long-term investigations, surprising effects were observed in the degradation of the coatings. In surface coatings made under anhydrous conditions according to the invention using nitro-fatty acids, the start of degradation was significantly delayed compared to a surface coating which had been prepared in an anhydrous manner with non-nitrated fatty acids. It was found that the nature and structural features of the hydrophobic cationic polyelectrolytes to which the fatty acids were applied had no effect on the beginning of degradation. Nonetheless, such a long-term stability was not present in the case of an anhydrously prepared surface coating with non-nitrated or nitrated fatty acids, where the surface had not been previously coated with a hydrophobic cationic polyelectrolyte. Degradation started here after 2-3 weeks and proceeded much faster than was the case after a coating according to the invention. For anhydrously prepared coatings prepared according to the invention, it was found that degradation starts at the earliest after 4 weeks under the selected experimental conditions.

In comparative studies on surface coatings in which no initial surface hydrophobization has taken place and/or hydrophilic cationic polyelectrolytes were applied from an aqueous medium and then nitrated or non-nitrated fatty acids were deposited in an anhydrous manner or in an aqueous medium onto such surfaces, it was found that with these methods the hydrophobicity of the resulting surfaces was comparable (water contact angle>90°) to a surface coating prepared according to the invention; however, the electrical isolation was 2-3 orders of magnitude lower when the coatings were prepared using hydrophilic cationic polyelectrolytes and under non-anhydrous conditions. It was found that with coatings prepared using hydrophilic cationic polyelectrolytes, degradation already took place after 1 to 2 weeks and continued very rapidly, so that after only a few days in a water bath the surface was nearly completely free of the coated material. As known in the art, it is not possible to prepare an anhydrous coating of surfaces with hydrophilic cationic polyelectrolytes. If a metal surface is only coated with hydrophilic cationic polyelectrolytes, there is no increase in the electrical surface resistance, but the insulation resistance was even reduced.

As previously stated, the provision of a material surface that does not contain ionic or osmotically active compounds or of a material that has not absorbed any water is of great importance in achieving the desired material surface properties. Furthermore, it has been shown that the adhesion force and the propagation of the electrolyte or polyelectrolyte layer according to the invention on a hydrophilic material surface can be increased or improved by a prior hydrophobic treatment step of the material, which can be done according to the prior art methods. Thus, in the inventive processes described herein, the solid materials provided in step a) preferably have a purified or a hydrophobized or a cleaned and hydrophobized material surface.

Thus, a preferred embodiment of the present invention is a process for producing a hydrophobic, biodegradable and insulating surface coating for corrosion and/or degradation retardation of solid materials comprising the following steps:

a) provision of a solid material comprising a cleaned or a hydrophobized or a cleaned and hydrophobicized material surface,

b) wetting of the surface of the solid material under anhydrous conditions with at least one hydrophobic cationic electrolyte or at least one hydrophobic cationic polyelectrolyte or a mixture comprising at least one hydrophobic cationic electrolyte and at least one hydrophobic cationic polyelectrolyte,

c) drying the surface,

d) wetting of the surface under anhydrous conditions with at least one carboxylic acid,

e) rinsing and drying the surface, and

f) obtaining the surface coating.

The results showed that achieving a hydrophobic surface with a water contact angle of ≥80° or >80°, more preferably ≥90° or >90°, more preferably ≥100° or >100°, more preferably ≥110° or >110°, more preferably ≥120° or >120°, more preferably ≥130° or >130°, more preferably ≥140° or >140°, even more preferably ≥150° or >150°, ≥160° or >160° and most preferably ≥170° or >170° and an electrical insulation resistance of ≥200 ohms/cm², preferably ≥300 ohms/cm², more preferably ≥400 ohms/cm², more preferably ≥500 ohms/cm², and most preferably of ≥1,000 ohms/cm² achieved by the coating layered construction according to the invention reliably prevents erosion/corrosion of the coated material.

Preferred is a process of the present invention for producing a biodegradable surface coating for inhibiting/retarding erosion/corrosion or degradation of solid materials, wherein the surface coating has surface hydrophobicity with a water contact angle of ≥80° or >80°, more preferably ≥90° or >90°, more preferably ≥100° or >100°, more preferably ≥110° or >110°, more preferably ≥120° or >120°, more preferably ≥130° or >130°, further preferably ≥140° or >140°, even more preferably ≥150° or >150°, ≥160° or >160° and most preferably ≥170° or >170°, and has an electrical insulation resistance of ≥200 ohms/cm², preferably ≥300 ohms/cm², more preferably ≥400 ohms/cm², more preferably ≥500 ohms/cm², and most preferably ≥1,000 ohms/cm². Also preferred is a surface coating for corrosion and degradation delay of solid materials available or obtained by the above mentioned method.

A further preferred surface coating for corrosion and/or degradation delay of solid materials comprises or consists of at least one carboxylic acid and at least one hydrophobic cationic electrolyte or hydrophobic cationic polyelectrolyte or a mixture comprising at least one hydrophobic cationic electrolyte and at least one hydrophobic cationic polyelectrolyte, wherein the surface coating is anhydrously prepared and applied and wherein a hydrophobic surface hydrophobicity with a water contact angle of >90° and an electrical insulation resistance of 500 ohms/cm² is obtained.

Preference is furthermore given to a process for producing a biodegradable surface coating for suppressing/delaying erosion/corrosion or degradation of solid materials, in which a single-layer or multi-layer layered construction consists of a hydrophobic cationic polyelectrolyte and a final coating with nitro-fatty acid or a mixture of nitro-fatty acids and non-fatty acids applied by means of a anhydrous application, whereby a hydrophobic surface with a water contact angle of ≥80, more preferably ≥90°, preferably ≥100°, more preferably ≥110° and most preferably ≥120° and electrical insulation resistance of ≥200 ohms/cm², preferably ≥300 ohms/cm², more preferably ≥400 ohms/cm², more preferably ≥500 ohms/cm², and most preferably ≥1,000 ohms/cm² is achieved. Likewise preferred is a surface coating for corrosion and degradation delay of solid materials available or obtained by the above mentioned method.

Further preferred is a surface coating for corrosion and/or degradation delay of solid materials comprising or consisting of at least one carboxylic acid and at least one hydrophobic cationic electrolyte or hydrophobic cationic polyelectrolyte or a mixture comprising at least one hydrophobic cationic electrolyte and at least one hydrophobic cationic polyelectrolyte, wherein a single-layer or multi-layer layered construction of a hydrophobic cationic polyelectrolyte and a final coating with nitro-fatty acid or a mixture of nitro-fatty acids and non-nitrated fatty acids by means of an anhydrous application, wherein the surface coating is prepared anhydrously, and wherein the surface coating has a surface hydrophobicity with a water contact angle of ≥80°, more preferably ≥90°, more preferably ≥100°, even more preferably ≥110°, and most preferably n of ≥120° and an electrical insulation resistance of ≥200 ohms/cm², preferably ≥300 ohms/cm², more preferably ≥400 ohms/cm, more preferably ≥500 ohms/cm², and most preferably ≥1,000 ohms/cm².

The surface coatings according to the invention, are further distinguished by the fact that degradation occurs spontaneously under physiological conditions because they are a physiosorptive assembly. Thus, a physiologically degradable/resorbable surface coating for corrosion/degradation delay can be provided by the methods of the invention.

It was then found that the start of degradation of surface coatings produced according to the invention is essentially dependent on whether a nitrated or non-nitrated fatty acid was used for the coating; however, the rate of degradation is essentially determined by the hydrophobicity and molecular weight of the cationic polyelectrolyte that was applied by an anhydrous application method. A considerable extension of the degradation time of the overall coating is given in particular when hydrophobic cationic polyelectrolytes having a K_{ow} of >0.3 are used. Furthermore, hydrophobic cationic polyelectrolytes, which were unbranched or only slightly branched and whose molecular weight was >10,000 Da, were associated with strongly retarded degradation rates. Such a condition was also present for a molecular weight of the hydrophobic cationic polyelectrolytes of 5,000 Da, if these were highly branched. Nevertheless, the rate of degradation also depended on whether the surface coatings produced according to the invention had been coated under anhydrous conditions with a nitrated or non-nitrated fatty acid and in case of using non-nitrated fatty acids degradation depended on the properties of those fatty acids, while such an effect was not recognizable when nitrated fatty acids were used. In the case of non-nitrated fatty acids, there was a degradation delay, especially when using linear fatty acids with a C-chain length of >5, with an optimum at a C-chain length of 14 to 18. By adding unsaturated fatty acids, the degradation delay decreased, however, even at a mixing ratio of 50% by weight between C16:0 and C18:1 fatty acids, a degradation delay lasting over a period of 4 weeks could be achieved. A significant increase in long-term stability over other fatty acids could then be documented for nitro-fatty acids with a C-chain length between 12 and 20. Thus, for example, when nitro-oleic acid alone was used, a doubling of the recognizable start of degradation compared to a coating prepared with oleic acid under anhydrous condition could be achieved. But even with mixtures of nitrated and non-nitrated fatty acids, there was a significant prolongation of the degradation time compared to the exclusive use of non-nitrated fatty acids. Correspondingly, the lifetime of the achieved surface hydrophobicity could be significantly prolonged by the use of nitro-fatty acids compared to the use of non-nitrated fatty acids of the same C-chain length.

The water contact angles measured in such coated templates after immersion for 8-weeks in an aqueous medium was still >90° when using a mixing ratio between nitrated and non-nitrated fatty acids of >0.25:1. Therefore, nitro-fatty acids cause a significant increase in the coherence of a coating with fatty acids. However, the highest long-term stability, which ensured freedom from degradation over a period of 8 weeks, was achieved when nitro fatty acids (C 18:1) were deposited under anhydrous conditions onto a hydrophobic cationic polyelectrolyte that had been applied under anhydrous conditions to a degradable material.

Preference is given to a process for producing a biodegradable surface coating to prevent/delay erosion/corrosion or degradation of solid materials and a degradation of the coating for 8 weeks, in which a single or multi-layered layer structure of a hydrophobic cationic polyelectrolyte and a final coating with at least one nitro-fatty acid or a non-nitrated fatty acid or a mixture of at least one nitro-fatty acids and at least one non-nitrated fatty acids by means of a water-free application is performed. In addition, a surface coating is obtainable or preferably obtained by the method described above.

Thus, a surface coating according to the invention described herein is preferred if it exhibits freedom from degradation for at least 8 weeks.

In good agreement with the isolation of material surfaces achieved by the coatings according to the invention, it was possible to document a prevention or delay in the degradation of the coated materials compared to uncoated materials in long-term corrosion investigations. Thus, for example, scaffolds made of a magnesium alloy with a coating system of dopamine, hydrophobicized polyethylene amine (25 kDa) and nitro-oleic acid, in which anhydrous deposition of the compounds was carried out, which were then fixated in a vessel model using a balloon catheter and the degradation that occurred under flow conditions of an aqueous medium were documented using sequential image analysis and the magnesium content in the aqueous medium was quantified. Comparing uncoated scaffolds with those which had only been coated with polyethylene amine (PEI), the time interval until reaching the onset of detectable corrosion was increased by 560% or by 250%, respectively. In addition, the time interval until complete dissolution of the scaffold was extended by 840% and 510%, respectively. A similar delay on initiation and complete dissolution could also be demonstrated for PLLA scaffolds when coated with a hydrophobic PEI (5 kDa highly branched) coating system and nitro-oleic acid (C18:1, also called nitro-oleic acid) coating system.

The resistance to degradation in an aqueous medium and the electrical insulation achieved could also be documented, for example, in metal surfaces coated according to the invention, such as, for example, steel alloys. In this case, the detection was carried out by means of a continuous determination of the electrical insulation resistance of the coating in an electrolyte bath and the measurement of the conductivity of the solution. The aqueous media contained among others acids or bases, enzymes or proteins in various concentrations, at room temperature and temperatures of up to 45° C. For the experiments on metallic materials which have a hydrophilic surface, application of an alkyl silane was performed first followed by an anhydrous application of hydrophobic (K_{ow} 1.3) polyalkylene polyamine (MW 8,000 Da) followed by anhydrous deposition of non-nitrated or nitrated fatty acids (C14-C20). A sole coating with the alkylsilane, followed by the hydrophobic cationic polyelectrolyte, achieved only a small increase in the electrical insulation resistance (0.8 ohms). Furthermore, after 3+/−2 days there was an increase in the conductivity of the storage solutions. A significant delay in the start of degradation (recognizable by a change in conductivity by 5%) or a defect in the insulation (insulation resistance drop by 100 ohm/cm²) occurred after 42+/−11 days in a coating with non-nitrated fatty acids and after 63+/−9 days for coatings with nitro-fatty acids.

The high and stable insulation resistance is also explained by a complete or almost complete freedom from defects of coated material surfaces prepared according to the invention. Since even in the presence of a high-resistance insulation resistance there can be small insulation gaps, which cause a leakage of current and thus can be the starting point of erosive/corrosive processes, a test must be carried out to ensure that there are no surface defects.

This takes place, for example, in an electrolyte bath by applying a DC voltage between the aqueous medium and the electrical connection to the test specimen. The metallic test materials were bonded by soldering to a platinum wire having with an insulating resin coating up to the junction. During the test, a 40V DC voltage was applied between the electrolyte bath and the electrical material connection.

For the comparative study, in one instance, stainless steel plates (10 cm²) were coated by means of spray coating with a polyurethane and a PLLA polymer, with a layer thickness of 20 and 100 m. For the other samples, stainless steel platelets were coated with the processes according to the invention, for example compounds 1, 3 and 7 according to Example 2, by applying 1 to 3 coats of nitro-oleic acid or nitro-linolenic acid and the native fatty acids.

In the case of the polymer coatings, there were of 5+/−3 defect sites per cm² for thin layers, and 3+/−2 defects per cm² for thicker layers. In contrast, there were between 1 and 2+/−1 defect sites per cm² after a single-layer coating with a hydrophobic cationic polyelectrolyte and a fatty acid with a C-chain length of >5, and with a surface water contact angle of >80° or ≥80°, and when using a multi-layered layer structure of the hydrophobic cationic polyelectrolytes, there were virtually no defect sites. In otherwise similar coatings in which the nitro-fatty acids had a C-chain length of 12 to 20, no defect sites were detectable in either a single-layer or a multi-layer coating with hydrophobic cationic polyelectrolytes.

Thus, in a preferred embodiment, a defect-free surface coating with a layer thickness of 15 nm to 10 μm is preferred.

Accordingly, a preferably biodegradable surface coating is obtainable by a method according to the invention which has a defect-free insulation of a material surface. Accordingly, a preferably biodegradable surface coating is obtainable by a method according to the invention with an electrically insulating material surface.

Therefore, a surface coating according to the invention described herein which is free or nearly free from insulation defects and has a surface contact angle of ≥80° or >80° is preferred.

Particular preference is therefore given to a surface coating according to the invention described herein, wherein a single or multi-layered construction of a hydrophobic cationic polyelectrolyte and a final coating with at least one nitro-fatty acid or a mixture of at least one nitro-fatty acids and at least one non-nitrated fatty acids, wherein the surface coating is free or is approximately free of insulation defects and a surface water contact angle of ≥80° or >80°, more preferably ≥90° or >90°, preferably ≥100° or >100°, more preferably ≥110° or >110° and most preferably ≥120° or >120°.

A preferred embodiment of the present invention is a surface coating for corrosion and/or degradation delay of solid materials comprising or consisting of at least one carboxylic acid, and at least one hydrophobic cationic electrolyte or hydrophobic cationic polyelectrolyte or a mixture comprising at least one hydrophobic cationic electrolyte and at least one hydrophobic cationic polyelectrolyte, wherein the surface coating is prepared in an anhydrous manner, and wherein the surface coating is free or nearly free of insulation defects and has a surface contact angle of >80°.

Further preferred is a method described herein for producing a hydrophobic, biodegradable and insulating surface coating for delay of corrosion and/or degradation of solid materials, wherein the surface coating is free or nearly free of insulation defects and exhibits a surface contact angle of >80°. In addition, a surface coating is preferably obtainable or obtained by the process just described.

Therefore, a preferred embodiment of the present invention is a process for producing a hydrophobic, biodegradable and insulating surface coating for corrosion and/or degradation delay of solid materials comprising the following steps:

a) providing a solid material,

b) wetting of the surface of the solid material under anhydrous conditions with at least one hydrophobic cationic electrolyte or at least one hydrophobic cationic polyelectrolyte or a mixture comprising at least one hydrophobic cationic electrolyte and at least one hydrophobic cationic polyelectrolyte,

c) drying the surface,

d) wetting of the surface under anhydrous conditions with at least one carboxylic acid,

e) rinsing and drying the surface,

f) obtaining the surface coating, and

wherein the surface coating is free or nearly free of insulation defects and has a surface contact angle of >80° or >80°.

Preferred is a process according to the invention described herein for producing a biodegradable surface coating to prevent/delay erosion/corrosion or degradation of solid materials comprising a single or multi-layered construction of a hydrophobic cationic polyelectrolyte and a final coating with nitro-fatty acid or a mixture of nitro-fatty acids and non-nitrated fatty acids by means of a water-free application that is free or almost free of insulation defects and exhibits a surface water contact angle of >80° or >80°. Preferably, a surface coating is available or obtained by the method described above.

Nearly free of insulation defects means that ≤2+/−1 defects per cm².

Further advantageous effects result from the degradation kinetics of the coatings according to the invention.

To examine different biological effects resulting from a coating composition according to the invention consisting of anhydrously applied hydrophobic polyelectrolytes and fatty acids, compared to a coating which was prepared with only non-nitrated or nitrated fatty acids, glass slides were coated accordingly and then fibroblasts were cultured upon these for 12 weeks. Cultures were analyzed at intervals of 14 days for vitality and structural changes of adherent cells. It was found that in a coating which was prepared with only non-nitrated fatty acids, there was a strong proliferation of adherent cells, which made it impossible to continue the cultivation after 4 weeks. When coated with nitro-oleic acid, there was a low proliferation rate of adherent cells within the first 4 weeks. In the further course, the proliferation rate increased and cell multilayers formed. In contrast, although confluency of adherent cells was a more rapidly achieved on a coating according to the invention with nitro-oleic acid as the final carboxylic acid layer, over the long-term there was only a sporadic formation, with completely closed cell coverage of the surface. The cells also differed morphologically: While the cells were predominantly configured in cell cultures in which there was a polygonal/dendritic proliferation tendency, cells adhering to/proliferating on a surface according to the invention had a spindle-shaped to rounded morphology. When examined at the respective time points, the cells were detached from the overlay by the addition of trypsin and the surface properties of the exposed coating areas were investigated. Furthermore, confocal laser microscopy was carried out after staining with a hydrophobic chromophore. It was found that the hydrophobic surface properties that were present after all coatings were originally prepared were no longer present, whereby this was the case after 2 weeks for a coating with non-nitrated fatty acids, after 4 weeks for coatings with nitrated fatty acids and after 10 weeks for a layered construction with a nitro-fatty acid as the outer boundary layer according to the invention. In cells grown on a coating of non-nitrated fatty acids, intracellular vacuoles and structural features of proliferating cells were found. In cells grown on a coating with nitro-oleic acid, such structural features did not exist until week 4 and were significantly less pronounced.

For cells grown on the surface coating of the present invention, such structural features never resulted. It could thus be shown that a layered construction according to the invention persists significantly longer on a surface on which cells adhere than is the case with a coating with carboxylic acids and is degraded in the course of time without triggering any identifiable tissue reaction.

Preferred is a method according to the invention described herein for producing a hydrophobic, biodegradable and insulating surface coating for the corrosion and/or degradation delay of solid materials, wherein a delayed biodegradation of the layer construction takes place without cell activation. Preference is furthermore given to a process for producing a biodegradable surface coating for suppressing/delaying erosion/corrosion or degradation of solid materials, in which a single-layer or multi-layer layer construction consists of a hydrophobic cationic polyelectrolyte and a final coating with a nitro-fatty acid or a mixture of nitro-fatty acids and non-fatty acids by means of a water-free application and in which delayed biodegradation of the layer structure takes place without cell activation.

Thus, biodegradation of the coatings of the invention takes place over time, which allows a subsequent degradation of resorbable materials. The advantage here is that due to the delayed degradation of the coated material, the cell composite is already solidly formed on/around this material and inflammatory processes that take place, for example, during the introduction or during the ingrowth of an implant, are already completed and therefore amplification of an inflammatory tissue reaction by degradation/corrosion products does not take place. Furthermore, it has been shown that the compounds which have been used for the layered construction according to the invention do not cause a recognizable pro-inflammatory effect during their degradation. Thus, in particular for hydrophobic cationic electrolytes applied in multiple layers, there was no sign of increased apoptosis of cells that settled or grew on such coatings. There was a relationship between the presence of an extracellular matrix which developed over time as an intermediate layer and a confluent, preferably single-layered cell structure which formed on a coating. Due to the affinity of cationic electrolytes to proteins, it can be assumed that the hydrophobic cationic electrolytes released or releasable in the context of degradation of the coating are incorporated into or bond to a developing extracellular matrix and become incorporated with it without triggering an immunological reaction.

The same can be assumed for non-nitrified and nitrified fatty acids. However, such fatty acids are also completely metabolized by cells; however, it is known that this likewise does not increase inflammatory processes. The layered construction according to the invention for preventing/delaying the erosion/corrosion or degradation of materials which come into contact with living cells or physiological media is thus biodegradable and biocompatible, with the simultaneous absence of pro-inflammatory effects during biodegradation. Thus, unlike the use of degradable polymers used to retard degradation of degradable materials of the prior art, it is not necessary to inhibit the proinflammatory effects resulting from polymer degradation by the simultaneous release of anti-inflammatory substances. On the other hand, in one embodiment, a layer structure according to the invention may contain substances which reduce or prevent inflammatory processes or cell proliferation. This is useful if delivery of these compounds into adjacent cells/tissues is supposed to take place before the degradation of the coating according to the invention, e.g. to influence the pathophysiological processes that exist through trauma or the underlying immunological processes. Therefore, the biodegradable layer structure according to the invention also ensures the release of biologically active compounds for immunomodulation in adjacent cells/tissues.

Thus, it could be shown that both hydrophilic and lipophilic compounds, which had been incorporated into a layer structure of hydrophobic cationic polyelectrolytes, could diffuse out of it and be detected in adjacent cell layers. It has also been shown that the release kinetics, in particular by the number of charge groups of the hydrophobic cationic polyelectrolytes, and their molecular weight and degree of branching, as well as a multi-layer layered construction can be controlled with a hydrophobic anionic polyelectrolyte. It could be shown that releasability/liberation of compounds which have been incorporated into a hydrophobic polyelectrolytic layered construction is possible without degradation of the layered construction. At the same time, the insulating properties of the coating according to the invention can be maintained. Thus, by the purely electrostatic cohesion of the coating components, a release of compounds and a degradation of the coating structure can be obtained independently of each other over time.

Therefore, a method according to the invention is preferred as described herein for producing a hydrophobic, biodegradable and insulating surface coating for the corrosion and/or degradation delay of solid materials, wherein in step b) at least one supportive compound, at least one active compound or a mixture containing at least one supportive compound and at least one active compound is used for wetting the surface of step a), the at least one supportive compound, the at least one active compound or the mixture containing the at least one supportive compound and the at least one active compound being introduced into the hydrophobic electrolyte layer in such a way that these diffuse out before the biodegradation of the layered construction or that these are released therefrom during degradation. Likewise, a surface coating is available or preferably obtained by the method described above.

At least one supportive and/or the at least one active compound may be admix to the at least one hydrophobic cationic electrolyte and/or to the at least one hydrophobic cationic polyelectrolyte and applied together therewith.

The at least one hydrophobic cationic electrolyte and/or the at least one hydrophobic cationic polyelectrolyte and/or the at least one anionic electrolyte and/or anionic polyelectrolyte may be admixed with the at least one supportive and/or the at least one active compound and applied together therewith.

A preferred embodiment is therefore a process for producing a hydrophobic, biodegradable and insulating surface coating for the corrosion and/or degradation delay of solid materials comprising the following steps:

a) providing a solid material,

b) wetting the surface of the solid material under anhydrous conditions with at least one hydrophobic cationic electrolyte or at least one hydrophobic cationic polyelectrolyte or a mixture comprising at least one hydrophobic cationic electrolyte and at least one hydrophobic cationic polyelectrolyte and further with at least one supportive compound, at least one active compound or a Mixture containing at least one supportive compound and at least one active compound,

c) drying the surface,

d) wetting of the surface under anhydrous conditions with at least one carboxylic acid,

e) rinsing and drying the surface, and

f) obtaining the surface coating.

Further preferred is a surface coating according to the invention described herein, wherein the surface coating further contains at least one supportive compound, at least one active compound or a mixture containing at least one supportive compound and at least one active compound, preferably the at least one supportive compound, at least one active compound or the mixture contains at least one supportive compound and at least one active compound introduced into the hydrophobic electrolyte layer in such a way that they may diffuse therefrom or are released from the layered construction prior to the biodegradation of the layered construction.

Preference is therefore further given to a process for producing a biodegradable surface coating for preventing/delaying erosion/corrosion or degradation of solid materials, in which a single- or multi-layered structure of a hydrophobic cationic polyelectrolyte and a final coating with nitro-fatty acid or a mixture of nitro-fatty acids and non-nitrated fatty acids by means of a water-free application takes place and in which active and/or supportive compounds are introduced into the hydrophobic electrolyte layer, preferably hydrophobic polyelectrolyte layer, and these diffuse/are released therefrom, before the biodegradation of the layer structure has taken place.

However, the incorporation or combination of degraded constituents in/with extracellular matrix proteins and/or their cellular incorporation, which takes place during the biodegradation of a coating according to the invention, also makes it possible to produce two-dimensional tissue composites on artificial material structures. Such properties of biodegradable coatings of implant materials are not known from the prior art.

Preference is given to a process for producing a biodegradable surface coating for preventing/delaying erosion/corrosion or degradation of solid materials, in which a single-layer or multi-layer construction of a hydrophobic cationic electrolyte and/or hydrophobic cationic polyelectrolyte and a final coating with at least one carboxylic acid is preferred with at least one nitro-fatty acid or a mixture of at least nitro-fatty acids and at least one non-nitrated fatty acids by means of an anhydrous application, due to which the compounds that are released during biodegradation are incorporated into an extracellular matrix or incorporated therein and/or are taken up by cells and/or are metabolized.

The coating according to the invention is characterized by its thin layered construction. These cannot be visualized by means of analytical imaging methods, such as scanning electron microscopy. It is also not possible to document their presence by spectral analytical methods. However, a layered structure of the coating according to the invention having a thickness between 5 and 15 nm was measured using EDX. An indirect detection method is confocal laser microscopy, in which the adherence of chromophores to a substrate can be determined at a lateral resolution of 200 nm. As a result, completeness of a coverage can be determined. It was found that the surfaces produced according to the invention were coated free of defects. There was also no defect in coatings prepared under anhydrous conditions with non-nitrated fatty acids after being placed in an aqueous medium for 4 weeks. Furthermore, the absence of defects in coatings made with nitrated fatty acids was significantly prolonged to 8 weeks.

Therefore, an anhydrously prepared nitro-fatty acid coating on anhydrously prepared hydrophobic cationic polyelectrolytes is a particularly preferred method for obtaining biocompatible degradable material/implant surfaces having hydrophobic surface properties.

Surprisingly, there were large differences in the biological effects that came apparent in cell experiments with templates that had been provided with different surface coatings. For this purpose, experiments with fibroblasts and human endothelial cells were carried out on coated planar preparations. Good adhesion of cells to hydrophobic cationic polyelectrolyte coatings prepared under anhydrous conditions was found. Compared to the untreated specimen surface, however, the proliferation rate of both cell lines was significantly increased when grown on a coating with hydrophobic cationic polyelectrolytes. In addition, the number of dead cells that were found after 2 days on the surfaces coated in this manner was significantly higher. Surfaces coated with hydrophobic cationic polyelectrolytes and non-nitrated fatty acids coated thereon had a moderate adhesion rate of the two cell lines. The influence of the C-chain length of the fatty acids was moderate. Proliferation of the cells corresponded to that achieved with the hydrophobic cationic polyelectrolyte surface coatings, with a significantly lower rate of cell death. In contrast, attachment of cells to surfaces made according to this invention in which nitro-fatty acids had been used was significantly increased.

However, the proliferation rate was significantly lower compared to a coating with non-nitrated fatty acids. The number of dead cells was also lower. It could further be shown that in a layered construction consisting of a hydrophobic cationic polyelectrolyte applied anhydrously, followed by an adsorbed monolayer of nitro-oleic acid applied anhydrously, a monolayer of endothelial cells that extended over the entire surface was present after 2 weeks, which did not proliferate further over the course of time. Such a result could not be achieved in a similar layered construction that was prepared with non-nitrated fatty acids, as the cells continued to proliferate in this setting.

Therefore, a layered construction/coating composition consisting of an anhydrous applied layer of hydrophobic cationic polyelectrolytes, followed by an application of fatty acids under anhydrous conditions, is a particularly preferred embodiment for obtaining a biocompatible surface for cell adherence. Particularly preferred is the use of nitro-fatty acids for the production of a biocompatible surface for adherence and controlled growth of cells on foreign surfaces.

Preferred is a process for producing a biodegradable surface coating for inhibiting/delaying erosion/corrosion or degradation of solid materials, comprising a single or multi-layered layered construction of a hydrophobic cationic polyelectrolyte and a final coating with nitro-fatty acid or a mixture of nitro-fatty acids and non-nitrated fatty acids by means of an anhydrous application takes place, whereby an adherence of cells with confluent planar tissue is made possible.

In further cell culture studies it could be shown that in the presence of cytokines which lead to cell proliferation under physiological and pathophysiological conditions, proliferation of fibroblasts and endothelial cells adhering to a coated material surface according to the invention was considerably lower than was in the case using a coating in which a non-anhydrous application of the hydrophobic cationic polyelectrolyte and/or the fatty acids had taken place or a hydrophilic cationic polyelectrolyte had been used. In an anhydrous application according to the invention of a hydrophobic cationic polyelectrolyte and of fatty acids, cell growth on such coated surfaces was significantly different if a non-nitrated or a nitrated fatty acid was used. When nitro-fatty acids were used, stimulation with cytokines resulted in a closed single-layered cell structure which did not proliferate any further.

In contrast, when using non-nitrated fatty acids, multilayer formation and uncontrolled cell proliferation occurred. Therefore, a coating according to the invention using nitro-fatty acids is a particularly preferred application for the formation of a monolayer full-surface cell assembly coverage.

Preference is given to a process for producing a biodegradable surface coating for preventing/delaying erosion/corrosion or degradation of solid materials, in which a single- or multi-layered construction of a hydrophobic cationic polyelectrolyte and a final coating with nitro-fatty acid or a mixture of nitro-fatty acids and non-nitrated fatty acids by means of a water-free application takes place, whereby the formation of a monolayer full-surface cell assembly of endothelial cells is made possible.

In further cell studies in which the culture medium contained blood serum, it was found that an extracellular matrix formed over a period of 4 weeks between a coated surface of the invention and the adjacent cell layer when using nitro-fatty acids coated on hydrophobic cationic electrolytes. This was not the case if the coated materials did not provide adequate protection against erosion/corrosion, and corrosion/erosion products could penetrate the coating. After being cultured for 10 weeks on a material surface coated with nitro-fatty acids according to the invention, no preparative separation of the extracellular matrix and compounds of the coating could be accomplished; however, although it was not possible to detect substances used for the coating, it must be assumed that these components have become part of the extracellular matrix and/or have been degraded and metabolized or have diffused into the aqueous nutrient medium. At this point, erosion/corrosion of the template materials was found if they consisted of a magnesium alloy or PLA. In further investigations, it has been found that if an extracellular matrix has formed between a surface of a degradable material coated according to the invention and a non-proliferating cell layer, progressive dissolution, for example of the magnesium alloy and the lactic acid polymer, occurs in the further course; it was found that in this situation there is no change in the phenotype of the adherent cells.

It was thus possible to show that a coating according to the invention prevents initial erosion/corrosion processes which otherwise occur on contact with water in the case of erodible/corrosion-sensitive materials and lead to corrosion products to remain locally which, after coating, favor permeability of those products and/or of water.

It has also been shown that very stable surface coatings can be produced with the coating method according to the invention, which have a high cohesion in an aqueous medium over 4 weeks and prevent penetration of water, whereby the surfaces remain hydrophobic and have good electrical insulation. Hydrophobic in this context means a water contact angle of >80° or ≥80°. Therefore, over a period of 4 weeks, there is no erosion/corrosion of degradable materials that are coated according to the invention. At the same time, the surface properties of materials coated according to the invention enable good cell adhesion, but a confluent cell layer in which there is no further proliferation of the cells is accomplished only with the use of nitro-fatty acids. Such a coating also allows formation of an extracellular matrix, during which degradation of the coating compounds also occurs. Furthermore, a significantly delayed degradation of degradable materials can be established by a coating method according to the invention. If a fused (closed) cell layer has already formed, degradation of metal alloys or organic polymers proceed slowly without relevant activation of the cells in contact with the degradation process. It has been found that these beneficial biological effects are dependent on the delayed degradation/corrosion, the stability of the hydrophobic surface in an aqueous medium, and the electrical insulation achieved with the coating.

These surface properties of materials coated according to the invention have further extremely advantageous effects. For example, it has been shown that virtually no activation of the human coagulation system occurs upon contact with a surface coated according to the invention. For this purpose, experiments with coagulation factors and whole blood were carried out under flow conditions. There was no relevant attachment of coagulation factors and/or platelets to the exposed surfaces even after 2 days of exposure. On the other hand, surfaces which had been coated only with cationic polyelectrolytes or only with fatty acids were clearly covered with blood constituents. For degradable coated materials, the surface coverage with blood components was also associated with the ion permeability of the coating. Therefore, prevention of the formation and/or passage of corrosion/erosion products of degradable materials is particularly preferred in order to reduce adhesion/aggregation of coagulation factors and/or platelets or to completely inhibit this, by provision of a surface coating according to the invention.

Preferred is a surface coating of the invention described herein for delayed corrosion and/or degradation of solid materials, whereby a complete or almost complete suppression of adhesion/aggregation of coagulation factors and/or platelets is achieved.

Further preferred is a process for producing a biodegradable surface coating to prevent/delay erosion/corrosion or degradation of solid materials, whereby complete or nearly complete suppression of adhesion/aggregation of coagulation factors and/or platelets is achieved.

Further preferred is a process for producing a biodegradable surface coating for inhibiting/delaying erosion/corrosion or degradation of solid materials, wherein a single or multi-layered construction of a hydrophobic cationic polyelectrolyte and a final coating with nitro-fatty acid or a mixture of nitro-fatty acids and not nitrated fatty acids by means of a water-free application, whereby a complete or almost complete suppression of adhesion/aggregation of coagulation factors and/or platelets is achieved.

Furthermore, it has been found that the electrical insulation of metallic surfaces can also be used to prevent the formation of a galvanic voltage, or a current flow. Thus, an electrical insulation and hydrophobization (water contact angle>90°) of a copper and a zinc element, in which an inventive application of an alkylsilane, followed by a coating with a hydrophobic cationic polyelectrolyte and subsequent deposition of nitro-erucic acid was carried out, can be achieved (>1,000 ohms/cm²). The elements were connected to a voltmeter via an electrical connection outside the water bath and were stored in an electrolytic bath for 4 weeks. During the course of this process, no current between the metal elements could be measured.

Preferred is a process for producing a biodegradable surface coating to prevent/delay erosion/corrosion or degradation of solid materials, thereby achieving electrical insulation.

Preference is furthermore given to a process for producing a biodegradable surface coating for preventing/delaying erosion/corrosion or degradation of solid materials, in which a single-layer or multi-layered construction comprising a hydrophobic cationic polyelectrolyte and a final coating with nitro-fatty acid or a mixture of nitro-fatty acids and non-fatty acids is used, which is applied by means of a water-free application method, whereby an electrical insulation is achieved.

In one embodiment, the surface to be coated is first rendered hydrophobic so as to be particularly effective on highly hydrophilic surfaces, such as glass, to increase the bonding stability to the hydrophobic cationic polyelectrolytes to be applied later. In one embodiment, this is done by methods of the prior art, such as by covalent or adsorptive bonding of the mostly amphiphilic compounds. Thus, in one embodiment, silanization, e.g. with an alkyl silane, takes place. Furthermore, in other embodiments, biogenic compounds such as dopamine may be physiosorptively adhered to a surface. The compounds used for the initial hydrophobization are preferably dissolved in an organic solvent and separated from the solvent phase on the surface to be coated. This can be done by known methods such as dip-coating or spray-coating. But there are also other forms of surface coating, which can be anhydrous, conceivable, such as deposition processes from a gas or vapor phase, which can be carried out with methods such as ALD or CVD. Preferred is the application of a monolayer. The application of a layer that is rendered hydrophobic can increase the adherence of hydrophobic cationic polyelectrolytes and at the same time lead to an improvement of the electrical insulation in a combined layer structure. On the other hand, it is also possible to apply compounds having positive and/or negative charge groups with a layer that is rendered hydrophobic in order, for example, to bind further compounds having opposite charge groups, preferably from an anhydrous solution. This can be particularly advantageous in particular when compounds are to be introduced into the layer structure, which are then to be released with great delay and as a result of degradation of the coating. In this case, the loading layer is applied directly to the layer that is rendered hydrophobic. It is preferred that this is likewise performed in an anhydrous manner, for example from a solvent phase. A procedure to render a surface that is hydrophobic is particularly preferred when the material surface is hydrophilic.

Therefore, it is preferable to render a surface hydrophobic if the water contact angle at the material surface is <40°, more preferably is <30°, and more preferably is <20° Preferred is a process for producing a biodegradable surface coating for inhibiting/delaying erosion/corrosion or degradation of solid materials, comprising a single or multi-layered layer structure of a hydrophobic cationic polyelectrolyte and a final coating with nitro-fatty acid or a mixture of nitro-fatty acids and non-nitrated fatty acids by means of a water-free application, in which the material surface is first rendered hydrophobic.

Therefore, preferably the processes according to the invention described herein after step a) comprise a step a2) cleaning, hydrophobizing or cleaning and hydrophobizing the surface of the solid material.

In a preferred embodiment of the present invention, the process for producing a hydrophobic, biodegradable and insulating surface coating for corrosion and/or degradation retardation of solid materials comprises the following steps:

a) providing a solid material;

a2) cleaning, hydrophobizing or cleaning and hydrophobizing the surface of the solid material;

b) wetting of the surface of the solid material under anhydrous conditions with at least one hydrophobic cationic electrolyte or at least one hydrophobic cationic polyelectrolyte or a mixture comprising at least one hydrophobic cationic electrolyte and at least one hydrophobic cationic polyelectrolyte,

c) drying the surface,

d) wetting of the surface under anhydrous conditions with at least one carboxylic acid,

e) rinsing and drying the surface,

f) obtaining the surface coating.

Accordingly, the inventive processes described herein preferably comprise after step a) a step a2) cleaning, hydrophobizing or cleaning and hydrophobizing the surface of the solid material if the water contact angle at the material surface is <40°, more preferably <30° and more preferably <20°.

In another embodiment, the application of one or more loading layer (s) with supportive and/or active compounds is carried out by first preparing a surface coating under anhydrous conditions with hydrophobic cationic polyelectrolytes and then applying thereto one or more compound(s) thereto.

An application of a mixture of one or more hydrophobic cationic polyelectrolytes together with an active and/or supportive compound can also be configured in a preferred embodiment. Furthermore, a layer structure which contains compounds to be released can also be achieved by means of an alternating layer-wise deposition of hydrophobic cationic polyelectrolytes and compounds that are to be released. In one embodiment, such a layered construction is achieved by the alternating application of hydrophobic cationic and hydrophobic anionic polyelectrolytes.

It has furthermore been found that controllable release systems for organic or inorganic compounds can also be prepared with a layered construction according to the invention. In one embodiment, hydrophobic compounds are used for this purpose, either directly or applying these to a surface that has been rendered hydrophobic (for example with dopamine or an alkylsilane) and then coating and/or after application of a hydrophobic cationic or anionic polyelectrolyte and alternately with a hydrophobic cationic and/or anionic polyelectrolytes, followed by deposition of fatty acids. The deposition of nitro-fatty acids is preferred. The compounds to be introduced into the layer structure are preferably hydrophobic and can be dissolved in an organic solvent and applied to the substrate surface from the solvent phase using known techniques, such as dip-coating or spray-coating. In another preferred embodiment, the compounds to be delivered are dissolved or suspended in one of the hydrophobic cationic or anionic polyelectrolytes and applied together with these on the surface by one of the methods described herein. In a preferred embodiment, the coating takes place according to the layer-by-layer process technology. In a further preferred embodiment, free fatty acids dissolved in an anhydrous medium are used for solubilizing compounds to be applied. This is particularly preferred if controlled release of supportive and/or active compounds is to take place with the layered construction according to the invention.

Preference is furthermore given to a biodegradable surface coating according to the invention, obtainable or obtained by a process described herein with controllable release of supportive compounds and/or active compounds.

It is preferred that the last coating layer, which prior to deposition of fatty acids on a compound-loaded surface, consists entirely or predominantly of a hydrophobic cationic electrolyte and/or hydrophobic cationic polyelectrolyte. Particularly preferred is the deposition of nitro-fatty acids as the final layer of a layered construction according to the invention for the release of compounds.

In a further process of the invention, the surface is wetted in a further step under anhydrous conditions with at least one hydrophobic anionic electrolyte and/or at least one hydrophobic anionic polyelectrolyte or a mixture comprising at least one hydrophobic anionic electrolyte and at least one hydrophobic anionic polyelectrolyte. In principle, this wetting can take place directly or indirectly on the surface of the solid material. Indirect means that the wetting is carried out on an already applied layer.

The steps b) to c) between the steps c) and d) can be performed twice or several times.

Preferred is a method according to the invention described herein, wherein between steps c) and d) the following steps b2) and c2) are carried out:

b2) water-free wetting of the surface with at least one hydrophobic anionic electrolyte and/or at least one hydrophobic anionic polyelectrolyte or a mixture comprising at least one hydrophobic anionic electrolyte and at least one hydrophobic anionic polyelectrolyte; c2) drying the surface.

The steps b2) to c2) between the steps c) and d) can be performed twice or several times.

Therefore, a preferred embodiment of the present invention is a process for producing a hydrophobic, biodegradable and insulating surface coating for corrosion and/or degradation retardation of solid materials comprising the following steps:

a) providing a solid material,

b) wetting of the surface of the solid material under anhydrous conditions with at least one hydrophobic cationic electrolyte or at least one hydrophobic cationic polyelectrolyte or a mixture comprising at least one hydrophobic cationic electrolyte and at least one hydrophobic cationic polyelectrolyte,

c) drying the surface,

b2) wetting of the surface under anhydrous conditions with at least one hydrophobic anionic electrolyte and/or at least one hydrophobic anionic polyelectrolyte or a mixture comprising at least one hydrophobic anionic electrolyte and at least one hydrophobic anionic polyelectrolyte;

c2) drying the surface;

d) wetting of the surface under anhydrous conditions with at least one carboxylic acid,

e) rinsing and drying the surface, and

f) obtaining the surface coating, Preferably, steps b) to c2) are carried out twice or several times.

Preference is furthermore given to a method according to the invention described herein, wherein in step b2) at least one supportive, at least one active compound or a mixture containing at least one supportive and at least one active compound is further used for wetting the surface.

Surprisingly, an inventive layered construction with supportive and/or active compounds provides that in particular hydrophobic compounds are dissolved out of the layered construction and released into a surrounding medium. It was particularly surprising that many of the hydrophobic but also hydrophilic compounds could be very well dissolved and formulated in an anhydrous solution of hydrophobic polyelectrolytes, so that in this way a well-adhering application of the compounds was made possible. Surprisingly, it has been found that the solubility of many supportive and active compounds can be significantly improved by the addition of nitro fatty acids in organic solvents. This has been true, for example, for active compounds, such as paclitaxel and everolimus, as well as for supportive compounds, such as cholesterol or carotenoids.

Surprisingly, nitro-fatty acids were also superior to non-nitrated fatty acids in dissolving compounds which can be completely dissolved in a solution thereof, when the melting point of the corresponding nitrated fatty acid was significantly higher than that of the non-nitrated fatty acid. The formulations prepared with nitro-fatty acids could be applied very uniformly in ultra-thin layers and could not be subsequently removed by aqueous or alcoholic solutions. In the case of a layered construction according to the invention, the release kinetics of compounds/active ingredients included herein correlated with that of the nitro fatty acids used, as well as with the concentration nitro fatty acids.

The release kinetics were also dependent on the concentration of the nitro-fatty acids used to solubilize the compounds/agents and on the number of layers of hydrophobic cationic and/or anionic polyelectrolytes. Surprisingly, despite the release of the compounds/active substances from the layered construction, there were no relevant changes in the physical properties of the coating: Surface hydrophobicity with a contact angle of >80° was still present even when more than half of the compounds/active substances had been released to the surrounding medium. The electrical conductivity did not change either. It must be assumed that at least part of the nitro-fatty acids incorporated into the layered construction for solubilization of compounds/agents have migrated to the outer layer consisting of fatty acids or nitro-fatty acids. Thus, in one embodiment, a coating method can be provided for uptake of compounds/active drugs with adjustable and controllable diffusion/release of these compounds/agents.

Preference is given to a process for producing a biodegradable surface coating for preventing/delaying erosion/corrosion or degradation of solid materials, in which a single- or multi-layered layered construction of a hydrophobic cationic polyelectrolyte and a final coating with nitro-fatty acid or a mixture of nitro-fatty acids and non-nitrated fatty acids by means of a water-free application takes place and at the same time supportive and/or active compounds can be incorporated in/on one or more layers of the hydrophobic cationic polyelectrolyte, whereby the compounds can be diffusively released.

Preference is given to a process in which hydrophilic and/or lipophilic supportive and/or active compounds with at least one carboxylic acid, preferably with at least one nitro-fatty acid or a mixture comprising or consisting of at least one nitro-fatty acid and at least one non-nitrated fatty acid, are mixed and thereby dissolved.

A preferred embodiment of the underlying invention therefore comprises methods for producing a hydrophobic, biodegradable and insulating surface coating for corrosion and/or degradation delay of solid materials comprising the following steps:

a) providing a solid material,

b) wetting of the surface of the solid material under anhydrous conditions with at least one hydrophobic cationic electrolyte or at least one hydrophobic cationic polyelectrolyte or a mixture comprising at least one hydrophobic cationic electrolyte and at least one hydrophobic cationic polyelectrolyte,

c) drying the surface,

d1) dissolving at least one supportive and/or at least one active compound in at least one carboxylic acid, preferably in at least one nitro-fatty acid or a mixture comprising or consisting of at least one nitro-fatty acid and/or at least one non-nitrated fatty acid,

d) wetting of the surface under anhydrous conditions with the solution from step d1),

e) rinsing and drying the surface,

f) obtaining the surface coating.

Preference is given to a process in which hydrophilic and/or lipophilic supportive and/or active compounds which are present in dissolved form together with nitro-fatty acids, are applied as a layer onto a coating comprising at least one hydrophobic cationic electrolyte or at least one hydrophobic cationic polyelectrolyte or a mixture comprising at least a hydrophobic cationic electrolyte and at least one hydrophobic cationic polyelectrolyte and/or are applied to a material with at least one hydrophobic cationic electrolyte or at least one hydrophobic cationic polyelectrolyte or a mixture comprising at least one hydrophobic cationic electrolyte and at least one hydrophobic cationic polyelectrolyte coating layer.

A release system of a surface coating for supportive and/or active compounds is preferred in which the diffusive release kinetics is adjusted by the quantitative ratio between the compound to be liberated and a nitro-fatty acid used for solubility/solubilization.

Surprisingly, it has been found that the duration of electrical insulation and/or prevention or retardation of erosion/degradation of a material surface coated according to the invention can be further extended by nitro-fatty acids applied in one or more layers consisting of hydrophobic cationic polyelectrolytes; this can be done simultaneously or the layers are applied separately from one another onto a layer of the layered construction.

In one embodiment, hydrophobic cationic polyelectrolytes are mixed with nitro-fatty acids. This is performed anhydrous and preferably in an organic solvent such as dichloromethane or pentane. The compound mixture is then applied in the solvent phase. After drying, the process can be repeated as often as desired. Even when hydrophobic cationic polyelectrolytes with a molecular weight of <5 kDa are used, coatings which are very stable and cannot be removed with water or with an alcohol are formed. In a preferred embodiment, the last layer is applied with a hydrophobic cationic polyelectrolyte in which no nitro-fatty acids are present. The final (outermost) layer is then applied by deposition of nitrated and/or non-nitrated fatty acids. The “final layer”, as defined herein, refers to the last applied layer. Starting from the surface to be coated, it is thus the outermost or last layer. Preferably, the surface coating comprises as the outermost layer carboxylic acid, preferably fatty acids, more preferably nitrated fatty acids, and further preferably a mixture comprising or consisting of nitrated fatty acids and carboxylic acids.

Evidence was provided that nitro-fatty acids, which had been used together with one of the compounds and the hydrophobic cationic polyelectrolyte to build up the layered construction, diffuse and adhere to the surface of the layer system, as nitro-fatty acids could be detected on the surfaces of a corresponding coating construction in which the final external surface covering was done exclusively with non-nitrated fatty acids. Thus, in one embodiment, a method can be provided wherein diffusion of the incorporated nitro fatty acids to the coating surface occurs by incorporation of nitro-fatty acids into a layered construction, with a hydrophobic cationic polyelectrolyte. Thus, a reservoir for mobile or mobilizable nitro-fatty acids can be prepared by introducing them alone or together with hydrophobic cationic polyelectrolytes in a coating composition, so that a redistribution of the nitro-fatty acids can take place via diffusive processes or concentration gradients. This is particularly advantageous since it allows defects in the outer layer, concerning fatty acids, to be compensated/replaced for/by nitro-fatty acids diffusing to the surface. Thus, a quasi self-healing hydrophobic surface coating can be provided. Of course, the fatty acids used for reservoir formation may also contain non-nitrated carboxylic acids.

Thus, “self-healing” or “quasi self-healing” as used herein means the ability of a coating comprising a plurality of layers to repair defects in the outermost layer by compounds from an inner layer by diffusion.

Thus, a preferred embodiment of the present invention is directed to a surface coating wherein the surface coating is self-healing.

Therefore, the object of the invention is also directed to processes for prolonging the provision of carboxylic acids/nitro-carboxylic acids on the coated material surface. As a result, both the degradation of the coating itself, as well as the degradation/erosion of the coated material can be further delayed. In addition, this also results in a facilitated transport of compounds from the coating structure to the coating surface and their delivery to a surrounding medium, as well as a provision of supportive compounds that counteract or prevent bio-film formation. Thus, the methods are also directed to reservoir formation, in particular for carboxylic acids and nitro-carboxylic acids, within the coating structure according to the invention.

Preferably, in a process according to the invention described herein, in step b) a mixture containing at least one carboxylic acid is preferably at least one fatty acid which is nitrated and/or non-nitrated and at least one hydrophobic cationic electrolyte, at least one hydrophobic cationic polyelectrolyte or a mixture of at least one hydrophobic cationic electrolyte and at least one hydrophobic cationic polyelectrolyte is used.

In one embodiment of the present invention, the process for producing a hydrophobic, biodegradable and insulating surface coating for corrosion and/or degradation retardation of solid materials comprises the following steps:

a) providing a solid material,

b) wetting the surface of the solid material under anhydrous conditions with a mixture containing at least one fatty acid which is nitrated and/or non-nitrated and at least one hydrophobic cationic electrolyte, at least one hydrophobic cationic polyelectrolyte or a mixture of at least one hydrophobic cationic electrolyte and at least one hydrophobic cationic polyelectrolyte,

c) drying the surface,

d) wetting of the surface under anhydrous conditions with at least one carboxylic acid,

e) rinsing and drying the surface, and

f) obtaining the surface coating.

In a further preferred process, in one of the processes according to the invention, in step b) fatty acids are mixed with the hydrophobic cationic electrolytes and/or hydrophobic cationic polyelectrolytes and applied and/or applied separately in a sequential manner to produce a reservoir formation for fatty acids and/or to solve supportive and/or active connections and to integrate them into the layer structure.

In a further preferred method, in one of the methods of the invention, in step b) fatty acids are mixed with the hydrophobic cationic electrolytes and/or hydrophobic cationic polyelectrolytes and applied and/or applied separately in sequential order to produce a reservoir for fatty acids and/or supportive and/or active compounds to dissolve and integrate into the layer structure and this process step is repeated 2 or more times and/or combined with other process steps.

Preference is given to a process for producing a biodegradable surface coating for suppressing/delaying erosion/corrosion or degradation of solid materials, in which a single- or multi-layered layered construction of a hydrophobic cationic and/or hydrophobic cationic polyelectrolyte and a final coating with carboxylic acid, preferably with at least a nitro-fatty acid or a mixture of at least one nitro-fatty acids and at least one non-nitrated fatty acid is/are introduced, by means of an anhydrous application and a reservoir for carboxylic acids in the layered construction is formed by the one or more carboxylic acids together with and/or on a layer of a hydrophobic cationic polyelectrolyte. Particularly preferred for reservoir formation is the use of at least one nitro-fatty acid.

Thus, in a method according to the invention described herein, the coating steps are chosen such that the reservoir is formed using carboxylic acids. For this purpose, after the application of the hydrophobic cationic layer and the layer of at least one carboxylic acid, a further layer sequence of the said layers can take place.

Furthermore, it is possible for a fatty acid to be applied together with the at least one hydrophobic cationic electrolyte and/or hydrophobic cationic polyelectrolyte and upon/above this a further layer comprising the at least one carboxylic acid or at least one further carboxylic acid is applied.

Surprisingly, it was found that nitro-fatty acids can be introduced in the layered construction in order to achieve formation of a reservoir within the organic polymers. For example, it has been shown that nitro-oleic acid which has been mixed anhydrously with a low molecular weight polyelectrolyte and dissolved in polyethylene glycol exhibited better dispersability, and can be incorporated homogeneously into the melt of polypropylene during an extrusion process and subsequently dried in the extruded product (e.g. thread material). After extrusion, nitro-fatty acids were detected at the surface. After intensive cleaning of the surfaces with organic solvents, it was no longer possible to detect nitro-fatty acids on the cleaned surfaces; however, after 2 days nitro-fatty acids could be detected again on the surface. Thus, in one embodiment, a reservoir for mobile nitro-fatty acids which can be incorporated onto and/or into organic polymers can be prepared under anhydrous conditions using a hydrophobic cationic polyelectrolyte with a nitro fatty acid and, thus, ultimately allowing diffusion of the nitro fatty acids from the organic polymers. It has also been shown that this reservoir formation can be used for carboxylic acids in order to prevent biofilm formation since the carboxylic acids present on the material surface and in particular the nitro-carboxylic acids have an anti-bacterial effect. Thus, it has also been possible to show that carboxylic acids, and in particular nitro-fatty acids, introduced together with a hydrophobic cationic polyelectrolyte into/onto a starting material, such as PP or PU, which is subsequently formed (thermally or mechanically) into a solid material, remain mobile/diffusible and reach the surface of the material by means of diffusion, where they arrange themselves. In a preferred embodiment, incorporation of a hydrophobic cationic electrolyte and/or hydrophobic cationic polyelectrolyte or a mixture thereof containing a carboxylic acid into/on a starting material/starting compound is converted into a solid piece of material by transformation and/or polymerization, whereby the introduced carboxylic acids/nitro-carboxylic remain mobile/diffusible within the solid material. Preference is given to formation of a reservoir in the solid material for carboxylic acids/nitro-carboxylic acids, which enables substitution of detached carboxylic acids/nitro-carboxylic acids by carboxylic acids/nitro-carboxylic acids diffusing (migrating) to the material surface.

Preference is given to a method for introducing carboxylic acids/nitro-carboxylic acids into a solid material which takes place during the production process and which ensures diffusion of the introduced carboxylic acids/nitro-carboxylic acids to the material surface.

Also preferred is a surface coating having a multilayered layer structure of a hydrophobic cationic electrolyte and/or hydrophobic cationic polyelectrolyte and a final layer comprising or consisting of at least one carboxylic acid, preferably at least one nitro-fatty acid or a mixture of at least one nitro-fatty acid and/or at least one non-nitrated fatty acid, wherein the application or layer formation takes place in each case in an anhydrous manner.

Carboxylic acids which have been applied as a surface coating on a medical device may diffuse into a body fluid or e.g. in vascular grafts or ingested or metabolized by cells, decreasing the number of carboxylic acids over time. By means of a surface coating containing a carboxylic acid reservoir, the carboxylic acids herein contained can be replenished to outermost layer once they are depleted therefrom. Furthermore, by using a carboxylic acid reservoir, the amount of carboxylic acids on the surface of a coating can be controlled or metered.

It has been found that the mixture of a hydrophobic cationic electrolyte or hydrophobic cationic polyelectrolyte with a carboxylic acid having a C-chain length of ≥6 carbon atoms results in a significant increase in the hydrophobicity of the mixture over that of the starting compound. This is recognizable, e.g. by a hydrophobic cationic electrolyte or polyelectrolyte that was previously soluble in an alcohol, which precipitated after addition of a carboxylic acid. Such mixtures can then be dissolved only in apolar solvents, such as toluene or pentane. This opens up further highly advantageous embodiments of the coating process. Thus, it has been found that multiple coatings can be made very effectively by coating as previously described with a hydrophobic cationic electrolyte or hydrophobic cationic polyelectrolyte alternating the coating procedure with a mixture of a hydrophobic cationic electrolyte or hydrophobic cationic polyelectrolyte and at least one carboxylic acid. In this case, the hydrophobic cationic electrolyte or hydrophobic cationic polyelectrolyte and the mixture of a hydrophobic cationic electrolyte or a hydrophobic cationic polyelectrolyte and the at least one carboxylic acid are each dissolved in a solvent having different polarity, such as methanol and pentane.

It has been found that, in contrast to a multiple deposition of a hydrophobic cationic electrolyte or hydrophobic cationic polyelectrolyte, in which the same organic solvent has been used each case, a significantly higher layer structure can be achieved with the same number of coating cycles, if a deposition of a hydrophobic cationic electrolyte or hydrophobic cationic polyelectrolyte and a mixture of a hydrophobic cationic electrolyte and on the other hand a hydrophobic cationic polyelectrolyte and at least one carboxylic acid, has been carried out alternately where the substances for the individual layer deposition have been dissolved in solvents of different polarity in each case. It is assumed that using an identical organic solvent for coating of at least one more coating cycle can lead to at least partial dissolution and detachment of already applied compounds. This is not the case if the deposition is carried out using a solvent mixture of different polarity.

Preference is given to a process in particular according to Example 2 in which a hydrophobic cationic electrolyte or a hydrophobic cationic polyelectrolyte and a mixture of a hydrophobic cationic electrolyte or hydrophobic cationic polyelectrolyte and at least one carboxylic acid is provided and in process step b), a hydrophobic cationic electrolyte or hydrophobic cationic polyelectrolyte or alternatively or in alternating sequence, a mixture of a hydrophobic cationic electrolyte or hydrophobic cationic polyelectrolyte and at least one carboxylic acid is used.

The order of application is in principle arbitrary, so that the hydrophobic cationic electrolyte or hydrophobic cationic polyelectrolyte or the mixture of a hydrophobic cationic electrolyte or alternatively a hydrophobic cationic polyelectrolyte and the at least one carboxylic acid can be deposited as a first layer on a surface to be coated. Preferably, another layered construction is applied in an alternating manner, but a layered construction is also possible in which first several layers of a hydrophobic cationic electrolyte or a hydrophobic cationic polyelectrolyte or a mixture of a hydrophobic cationic electrolyte or hydrophobic cationic polyelectrolyte and the at least one carboxylic acid are applied. This may be advantageous in particular when introducing active or supportive compounds. It has also been found that deposition of carboxylic acids, including nitro-fatty acids onto a coating using a hydrophobic cationic electrolyte or hydrophobic cationic polyelectrolyte have higher stability against dissolution and removal of the hydrophobic cationic electrolyte or hydrophobic cationic polyelectrolyte from the surface upon bringing the surface in contact with an apolar solvent.

Therefore, in a further preferred embodiment, following a deposition of a hydrophobic cationic electrolyte or a hydrophobic cationic polyelectrolyte in process step b) and drying in process step c), a deposition of at least one carboxylic acid, including nitro-fatty acids, takes place in process step b3). The carboxylic acid can in principle be applied in concentrated form, but for a single-layered construction solution in an organic solvent is preferred. Preferred organic solvents which are used in process step b3) are alcohols. Following process step b3), drying takes place. If a further coating is desired, a further coating cycle is carried out with a mixture of a hydrophobic cationic electrolyte or a hydrophobic cationic polyelectrolyte and at least one carboxylic acid with the mixture preferably being present in an apolar solvent.

Preference is given to a process in particular according to Example 2 in which, in process step b), a hydrophobic cationic electrolyte or a hydrophobic cationic polyelectrolyte or a mixture of a hydrophobic cationic electrolyte or hydrophobic cationic polyelectrolyte is used for wetting the surface of the solid material and then in step c) drying takes place and subsequently, in step b3), a wetting of the surface, obtainable after step c), takes place with a nitrated and/or non-nitrated carboxylic acid.

In accordance with the above observations that carboxylic acids, including nitro-fatty acids introduced for the purpose of reservoir formation as an intermediate layer alone or together with an active or supportive compound diffuse to the surface of the coating very likely due to a diffusion gradient, in a layered construction in which the fatty acid has been applied either in the form of one of the aforementioned mixtures or as a single layer, whereby over time these fatty acids are present on the coating surface. Thus, in principle, a final coating layer, performed with a hydrophobic cationic electrolyte or a hydrophobic cationic polyelectrolyte and with the at least one carboxylic acid according to step d), can be dispensed with provided that at least one carboxylic acid has been introduced in at least one layer in the overall coating construction procedure.

However, in order to guarantee an immediate provision of the at least one carboxylic acid, in particular nitro-fatty acids, on the surface of the coating, the embodiment of step d) according to the invention is advantageous. It has been shown that it is advantageous if the last layer before the execution of step d) is a hydrophobic cationic electrolyte or a hydrophobic cationic polyelectrolyte and a mixture of a hydrophobic cationic electrolyte or a hydrophobic cationic polyelectrolyte without an addition of a carboxylic acid.

It has furthermore been found that the hydrophobic cationic electrolyte or a hydrophobic cationic polyelectrolyte and mixtures of these tend to form clusters on surfaces. This tendency is more pronounced on hydrophilic surfaces. The clustering can lead to shrinkage phenomena of the coating having a map-like configuration, which can cause defects in the coating. This is the case in particular when using high molecular weight polyelectrolytes and high concentrations of these. It has been found that such cluster formation can be counteracted by various measures. Thus, on the one hand, the surface to be coated can first be rendered hydrophobic. Further, a low concentration and a multi-layer deposition of a hydrophobic cationic electrolyte or hydrophobic cationic polyelectrolyte may be used. An alternating coating with a hydrophobic cationic electrolyte or a hydrophobic cationic polyelectrolyte with a mixture of a hydrophobic cationic electrolyte or hydrophobic cationic polyelectrolyte and at least one carboxylic acid also results in a more uniform surface distribution. Furthermore, however, a subsequent spreading of clustered compounds can also be achieved. For this purpose, methods of the prior art can be used. It could be shown that spreading is possible by treatment with oxygen or argon plasma. An improvement of the spread can be recognized in that the scattering is significantly reduced in repeated water contact angle measurements. But also by means of electro-chemical determination of defects, the number of defects is reduced. Spreading can also be carried out by thermal treatment following drying and by ultrasound treatment that can be performed after application.

Surprisingly, a surface coating according to the invention has an antimicrobial effect. Thus, it was found that bacteria which are very often causative for the formation of an infectious deposit on implants (biofilm) neither adhered to this nor grew on it despite immediate exposure to the coated material surfaces, as long as there was no evidence of incipient degradation of coated samples.

Repeated studies over longer periods of time have found that nitro-fatty acid coatings provide significantly longer freedom from bacterial colonization than coatings in which the corresponding native fatty acid has been use. It has further been documented that incorporation of carboxylic acids into a coating layer consisting of a hydrophobic cationic electrolyte or polyelectrolyte results in formation of a reservoir of this fatty acid, allowing it to distributed through the coating layers and to be deposited on the surface of the coating. Thus, there is a reoccupation of a surface cationic binding site of the cationic electrolyte, as the carboxylic acids are released into a surrounding medium. Since it could be shown that nitro-fatty acids have a significantly greater antibacterial effect than the corresponding native fatty acid, it is thus possible to provide a surface coating in which anti-microbially active carboxylic acids and, in particular, nitro-carboxylic acids are provided, which are introduced in the form of a reservoir into the coating and thus ensure prolonged freedom from biofilm formation.

Preference is given to a process for surface coating in which a reservoir formation for anti-microbially active carboxylic acids/nitro-carboxylic acids is formed during the production process.

A surface coating with a long-term anti-microbial effect is preferred.

Preference is given to a surface coating which has an anti-microbial long-term effect. Preferred is a surface coating in which a long-term anti-microbial effect is achieved by carboxylic acids/nitro-carboxylic acids within the coating that can diffuse to the material surface.

The embodiments of the method according to the invention make it possible to shield a material from an aqueous medium in a very simple and cost-effective manner. In this case, an initialization of corrosive processes by contact with water can be completely prevented even during the application. With the compounds that can be used, a long-lasting sealing of surfaces which is biodegradable can be ensured, and by choosing suitable and biodegradable compounds of the hydrophobic cationic polyelectrolytes and the carboxylic acids, the time of dissolution of the surface coating can be controlled.

This degradation does not lead to unwanted cell/tissue reactions. This is also due to the low thickness of the surface coating according to the invention, which also causes an improved advanceability (due to a lower frictional resistance at the surface), in particular of vascular implants in the vascular system. The non-covalent layer structure allows the greatest possible flexibility between the bonds or the layers with one another, so that it is possible even with a geometric change of the coated material or its expansion that the compounds used for the coating adapt to the changed shape or dimensions without defects. The surfaces coated according to the invention allow very good adherence of cells and are therefore suitable for cell homing. The use of nitro-fatty acids causes controlled growth of adherent cells and allows formation of a monolayer confluent cell/tissue network. Therefore, surface coatings according to the invention using nitro-fatty acids are also very suitable for applications e.g. in the field of “tissue engineering” or endothelialization of endovascular implants or endothelialization of surfaces in plastic surgery. Further, a multi-layered layered construction with hydrophobic polyelectrolytes is possible, which allows the introduction of other compounds, such as hormones, antioxidants or drugs, which can be electrostatically bound via the selection of suitable hydrophobic cationic and/or anionic polyelectrolytes and thus diffusely released in a controlled manner. Furthermore, water-free mixtures of hydrophobic cationic polyelectrolytes and nitro-fatty acids can be used to prepare reservoir systems which enable a diffusive distribution and release of nitro-fatty acids and can be incorporated into a superficial layer structure or into polymer structures. Thus, the methods of the invention also provide a controllable release system for compounds/drugs/nitro-fatty acids from surface coatings. This reservoir formation can be used, in particular, for loading anti-microbially active carboxylic acids/nitro-carboxylic acids, in order to ensure a long-lasting replenishment of carboxylic acids/nitro-carboxylic acids that diffuse out of the surface layer, thereby enabling long-lasting protection against biofilm formation.

Thus, a surface coating of the invention described herein is obtainable or obtained by a method of the invention for use in cell homing and for confluent cell growth.

### Definitions

### Anhydrous

“Anhydrous” means that the at least one hydrophobic cationic electrolyte to be applied and/or at least one hydrophobic cationic polyelectrolyte or the at least one nitro-fatty acid or at least one non-nitrated fatty acid or a mixture of the at least one nitro-fatty acid or at least one non-nitrated fatty acid and their solutions has a maximum total water content of 1000 ppm. If, for example, a solution of the hydrophobic cationic polyelectrolyte is prepared with a solvent, the solution is anhydrous if at most 1,000 ppm of water is present in the solution.

Preferably, the total amount of water is at most 750 ppm, more preferably at most 500 ppm, more preferably at most 250 ppm, more preferably at most 150 ppm, more preferably at most 100 ppm, more preferably at most 90 ppm, more preferably at most 80 ppm, more preferably of at most 70 ppm, more preferably at most 60 ppm, more preferably at most 50 ppm, more preferably at most 40 ppm, more preferably at most 30 ppm, more preferably at most 20 ppm and most preferably at most 10 ppm.

### Erosion/Corrosion/Degradation

The terms “erosion”, “corrosion” and “degradation”, which are also used interchangeably, refer to one or more subsequent and/or parallel processes in which, due to hydrolysis and/or oxidation, they increase a dissolution of atoms/ions and/or molecules and/or particles from a composite material and/or in which atoms/ions and/or compounds are changed by a physico-chemical/chemical reaction as a result of contact with an aqueous medium. As a result, reaction and/or degradation products may arise, which are in the form of a loose composite and/or diffuse into a surrounding medium. Examples include the oxidation of iron with the formation of iron ions and iron oxides or the hydrolytic cleavage of lactic acid polymer chains to release lactic acid molecules or the hydrolytic release of magnesium ions from a metal lattice structure with the release of hydrogen. Consequently, the erosion, corrosion or degradation products are those elements, ions or compounds which are formed or released during an erosion, corrosion or degradation process.

### K_{ow}

The term K_{ow} as used herein means the distribution quotient of a compound between an octanol and a water phase in a non-ionized state.

The K_{ow} or P value refers only to one species of a substance:

$${K}_{ow} = P = \frac{{c}_{o}^{{S}_{i}}}{{c}_{w}^{{S}_{i}}}$$

Whereas: cₒ^{s}^{ᵢ} represents the concentration of the species/substance in the octanol-rich phase and

c_{w}^{s}^{ᵢ} represents the concentration of the species/substance in the water-rich phase.

### Biodegradable

The terms “resorbable” or “degradable” or “biodegradable” or “bio-degradable” or biologically degradable refer to the fact that the human or an animal organism is able to dissolve the surface coating or the solid material within a certain period of time, so that atoms, ions or molecules that become present, which are dissolved in the blood or other body fluids can be metabolized and/or excreted.

Freedom from Degradation.

Stability/resistance from degradation can be arbitrarily designed by the methods disclosed herein. In the absolute sense, a freedom from degradation is achieved over a set period of time. The term “freedom from degradation” as defined herein means that there is no change in at least 2 of the following properties over a defined period of time: surface hydrophobicity, adhesion behavior of living cells, adhesion behavior of plasmatic blood components, leaching of corrosion/corrosion products of the coated material into a surrounding aqueous medium, electrical insulation, mechanical integrity/stability of the coated material.

The properties are tested by placing the coated material in a suitable aqueous medium for the period to be tested. Electrical insulation is present at an electrical resistance of >200 ohm/cm², preferably >300 ohm/cm², more preferably >400 ohm/cm², more preferably ≥500 ohm/cm², even more preferably >1,000 ohm/cm², more preferably >1,100 ohm/cm², even more preferably >1,200 ohm/cm², even more preferably >1,300 ohm/cm², even more preferably >1,400 ohm/cm², even more preferably >1,500 ohm/cm², even more preferably >1,600 ohm/cm², even more preferably >1,800 ohm/cm², even more preferably >1,900 ohm/cm², even more preferably >2,000 ohm/cm², even more preferably >2,500 ohm/cm², and most preferably even more ≥3,000 ohm/cm².

The change in the abovementioned properties is preferably <10%, more preferably <5%, more preferably <3% and more preferably <1%, based on the starting state before application or the test investigation. In this case, there is no degradation preferably over a period of 4 weeks, more preferably 8 weeks, and even more preferably 12 weeks.

In this respect, the degradation stability also causes a degradation delay/retardation, which can be quantified using the aforementioned parameters. On the one hand a delay of degradation of the surface coating itself is meant. This refers to the loss/release of carboxylic acids/nitro-carboxylic acids as compared to a single surface coverage on a material. However, the term “degradation delay” also refers to the erosion/corrosion of the coated material, which occurs with a time delay compared to a sole coating with a carboxylic acid/nitro-carboxylic acid. Preferred is a time delay of more than 14 days, more preferably >30 days, more preferably >45 days and even more preferably >60 days, compared to a sole surface coating with the corresponding fatty acid/nitro-fatty acid.

### Medical Instruments, Implant Materials and Wound Materials

The terms “medical device” or “medical devices” are used herein as generic terms including any implants, natural and artificial grafts, sutures and bandage materials, as well as parts of medical devices such as catheters. Medical products, which also include cosmetic or partially cosmetic and partially medical implants, are preferably medical devices, more preferably implants that are temporarily or permanently introduced into the organism and medical objects that come into contact with cells/tissues, such as wound materials, suture materials, wound and body cavity closure systems, biological grafts, artificial grafts, biological implants, artificial implants, artificial blood vessels, natural blood vessels, blood conductors, blood pumps, dialyzers, dialysis machines, vascular prostheses, vascular supports, heart valves, artificial hearts, vascular clamps, autologous implants, bone implants, intraocular lenses, shunts, dental implants, infusion tubes, medical cuffs, bandages, medical clamps, pumps, pacemakers, laboratory gloves, medical scissors, medical cutlery, needles, cannulas, endoprostheses, exoprostheses, scalpels, lancets, soft tissue implants, breast implants, facial implants, catheters, guide wires, ports, stents, catheter balloons and catheter balloons with attached stent, and vascular grafts. The surface coatings according to the invention are thus suitable for all medical devices or medical devices that come into contact with cells/tissues/organs temporarily or permanently and that this contact may lead to irritation of cells/tissues/organs as a result of this contact, causing undesirable reactions (as described below or on page 8 above) of said structures. This includes medical or cosmetic procedures that have similar properties.

The medical instruments, implant materials and wound materials referred to herein are all made of solid materials, of which at least a part of the surface(s) are or may be in temporary and/or permanent contact with cells/tissues/fluids of a human or animal body/organism or also with ex vivo tissue associations or contact individual cells or can get in contact.

In the methods of the invention described herein, the solid materials are preferably a medical device, particularly a medical device that may come into contact with cells, tissues, biological fluids, or a combination of at least two of these.

Furthermore, another aspect of the present invention is a medical product having a coating comprising or consisting of at least one carboxylic acid; and at least one hydrophobic cationic electrolyte or hydrophobic cationic polyelectrolyte or a mixture comprising at least one hydrophobic cationic electrolyte and at least one hydrophobic cationic polyelectrolyte, wherein the coating is prepared under anhydrous conditions.

Thus, another aspect of the underlying invention is a medical product having a hydrophobic, biodegradable, and insulating surface coating obtainable by a method according to the invention described herein. The medical products can be implants, in particular long-term or short-term implants. It is also possible to equip permanent long-term implants with a surface coating according to the invention. For example, Co/Ni stainless steel alloys can be provided with a surface coating according to the invention.

This prevents the diffusion of allergenic components, especially nickel, and thus an allergic reaction. Even in the case of permanent long-term implants in which a bioresorbable surface coating does not last the entire life of said implant, it is important to prevent additional exposure to allergy-causing substances in addition to the inflammatory influences in the first weeks after implantation.

The solid materials referred to herein may be hard or soft, solid or flexible and formable (moldable). This does not include materials that are liquid, gelatinous or pasty. These include, but are not limited to:

Soft tissue implants such as silicone implants, joint implants, cartilage implants, natural or artificial (e.g. Dacron) tissue implants and grafts, autogenous tissue implants, intraocular lenses, surgical adhesion barriers, nerve regeneration channels, birth control devices, shunts, tissue scaffolds such as pericardial tissue, tissue-based matrices, dental devices and dental implants, drug infusion tubes, cuffs, drainage devices (e.g. for eyes, lungs, abdomen, urine, jaw), tubes (endotracheal, tracheostomy) and tubing (also for extracorporeal circulation), surgical mesh, ligatures, sutures, staples, wires, pins, nails and screws, protective material, foams, pedicles, films, implantable electrical stimulators, pumps, ports, reservoirs, injection catheters or stimulation or sensing electrodes/probes, wound coatings/dressings, sutures, membranes, rings or sleeves, surgical instruments such as scalpels, lancets, scissors, tweezers or hooks, clinical gloves, hypodermic needles, endoprostheses and exoprostheses. Vascular implants, arterial stents, including scaffolds, stents and flow diverts, as well as coils. Furthermore, osteosynthetic materials (materials for osteosynthesis), catheters (including infusion cannulas), wound dressings or wound dressings, foams, absorbers, gauze, bandages. Wound closing material such as sutures, filaments, clips, wires and the like. Also orthopedic prostheses, dental implants, fixators and drains. A particularly preferred embodiment of the present invention relates to a method according to the invention, wherein the solid material are preferably arterial support materials, in particular stents.

A further embodiment of the present invention is thus an arterial stent with a surface coating according to the invention, which is preferably biodegradable, obtainable by a method according to the invention described herein.

A further embodiment of the present invention is thus an arterial stent with a hydrophobic, biodegradable and insulating surface coating, obtainable by a method according to the invention described herein.

A further embodiment of the present invention is thus a stent having a surface coating according to the invention preferably biodegradable obtainable by a method according to the invention described herein.

A further embodiment of the present invention is thus a stent with a hydrophobic, biodegradable and insulating surface coating obtainable by a method according to the invention described herein.

A further preferred embodiment is directed to an arterial stent having a surface coating comprising at least one carboxylic acid; and at least one hydrophobic cationic electrolyte or a hydrophobic cationic polyelectrolyte or a mixture comprising at least one hydrophobic cationic electrolyte and at least one hydrophobic cationic polyelectrolyte, wherein the surface coating is made under anhydrous conditions.

A further preferred embodiment is directed to a stent having a surface coating comprising at least one carboxylic acid; and at least one hydrophobic cationic electrolyte or a hydrophobic cationic polyelectrolyte or a mixture comprising at least one hydrophobic cationic electrolyte and at least one hydrophobic cationic polyelectrolyte, wherein the surface coating is made under anhydrous conditions.

Therefore, one embodiment of the underlying invention is directed to a medical product having a coating comprising or consisting of at least one carboxylic acid; and at least one hydrophobic cationic electrolyte or hydrophobic cationic polyelectrolyte or a mixture comprising at least one hydrophobic cationic electrolyte and at least one hydrophobic cationic polyelectrolyte, wherein the coating is prepared under anhydrous conditions, wherein the medical product is a soft tissue implant, such as silicone implants, joint implants, cartilage implants, natural or artificial (e.g. Dacron) tissue implants and grafts and vascular prostheses, autogenous tissue implants, intraocular lenses, surgical adhesion barriers, nerve regeneration conducts, birth control devices, shunts, tissue scaffolds such as pericardial tissue, tissue-based matrices, dental devices and dental implants, drug infusion tubes, cuffs, drainage devices (e.g. for eye, lung, abdomen, urine, jaw) tubes (endotracheal, tracheostomy) and tubes (also for extracorporeal circulation), surgical meshes, ligatures, sutures, staples, wires, pins, nails and screws, masking material, foams, pedicles, films, implantable electrical stimulators, pumps, ports, reservoirs, injection catheters or stimulation or sensing electrodes/probes, wound coatings, sutures, membranes, rings or sleeves, surgical instruments, such as scalpels, lancets, scissors, tweezers or hooks, clinical gloves, hypodermic needles, endoprostheses and exoprostheses, vascular implants, including scaffolds, stents and flow diverts, and coils, osteosynthetic materials (materials for osteosynthesis), catheters (including infusion cannulas), wound dressings, foams, absorbers, gauze, bandages. Suture materials such as sutures, filaments, clips, wires and the like or orthopedic prostheses, dental implants, fixators and drains. Preference is given to vascular implants, in particular a stent in the sense of a vascular support and a so-called scaffold, which is generally bioresorbable. However, this also includes so-called flow diverter and implantable sleeves.

### Substrate Materials

The terms “substrate materials”, “materials”, “solid materials” and “templates” are used interchangeably herein and refer to the material/work piece that is to be surface-coated.

In principle, all materials available from the prior art can be used. However, it is preferred that the materials are materials used in medical products, most preferably materials used for medical instruments, implant and wound materials. These may be inorganic or organic as well as composite materials. The inorganic materials may be, for example, metals or metal alloys, sintered silicon, zirconium, aluminum compounds, as well as light metals such as aluminum, including their oxide forms.

Metal alloys, from which vascular implants, such as stents are made, for example, 316L stainless steel based. “316L” is a name of the American Iron and Steel Institute (ASI). This alloy is further subdivided in Europe according to EN10088 into three alloys: X2CrNiMo17-12-2, X2CrNiMo18-14-3 and X2CrNiMo17-12-3. They differ in their content of molybdenum, which varies between 2% and 2.5%.

The alloy consists mainly of iron (base), chromium (18%), nickel (8%) and molybdenum (2-2.5%). A further development of stent materials include cobalt-chromium alloys, such as L605. This alloy consists of cobalt (base), chromium (19-21%), tungsten (14-16%), nickel (9-11%), iron (max 3%) and manganese (1-2%) and low amounts of silicon (max 0.4%), carbon (0.05-0.15%) and sulfur (max 0.03%). The DIN designation of the alloy is CoCr20W15Ni, and Co-20Cr-15 W-10Ni. Furthermore magnesium alloys are also particularly preferred. Such an alloy is known e.g. as Resoloy.

Furthermore, inorganic compounds also include silicon-based materials, such as silicone. The organic materials are carbon-based compounds which are in the form of natural polymers such as cellulose or latex or in the form of synthetic polymers such as polyurethane, polypropylene, acrylates, polyamides or polyesters.

The preferred materials can be durable and non-susceptible to corrosion, easily degradable and self-dissolving.

In the inventive methods and coated medical products described herein, it is particularly preferred that the solid material is a corrodible and/or degradable material.

Also particularly preferred is a medical product according to the invention with a coating according to the invention, wherein the medical product consists of a corrodible and/or degradable material.

The corrodible and/or degradable materials include, but are not limited to, polymers such as PLLA (poly-L-lactide) and polycarbonates comprising iodinated desaminotyrosyl-tyrosine-ethylester.

Biodegradable Polymers can be Classified into

(i) natural polysaccharides (e.g. starch, cellulose, chitin, chitosan, hyaluronan, pectin) and naturally occurring proteins (e.g. fibrin, gelatin, collagen, laminin and fibronectin) or coupling products of natural materials with synthetically produced compounds such as synthetic polymers (e.g. poly (vinyl alcohol), poly (ethylene oxide), poly (vinyl pyrrolidone))

(ii) semisynthetic (e.g., chemically modified natural polymers such as chitosan), and

(iii) synthetic [e.g. poly (lactic acid) (PLA), poly (glycolic acid) (PGA), poly (lactic-co-glycolic acid) (PLGA), poly (ε-caprolactone) (PCL), poly (dioxanone) (PDO), poly (anhydride), poly (trimethylene carbonate), poly (ortho ester), poly (phosphazene), poly (cyano acrylate) and poly (hydroxyl butyrate).

The materials may be solid or flexible or soft as long as it is a solid material. The outer shape is irrelevant, as long as it is possible to ensure a coating that is closed on all sides using one of the disclosed coating methods. The materials are nonimpregnable or nonabsorbent. Absorbent tissues and membranes can also be used.

### Hydrophobic Cationic Electrolytes and Hydrophobic Cationic Polyelectrolytes

Polyelectrolytes are polymers which carry many ionic or ionizable groups. Depending on the nature of the ionizable groups, they may be present as polybase or polycation and as polyacid or polyanion. Cationic polyelectrolytes are thus polymers which carry ionic or ionizable groups, i.e., they are present either as polybase or polycation. Accordingly, electrolytes are compounds which carry one or more ionic or ionizable groups and, depending on the nature of the ionizable groups, are present as a base or cation. The hydrophobic cationic electrolytes and polyelectrolytes according to the invention are characterized by one or more cationic charged groups which are ionizable or by one or more basic groups which are ionizable, a carbon compound which contains no or only a very small proportion (<2% by weight) of hydrophilic groups (e.g. OH groups) and has a K_{ow} of >0.3 or ≥0.3. A polyelectrolyte carries at least two cationic charged groups or at least two basic groups which are ionizable and preferably a carbon-containing quaternary nitrogen compound. Preferably, at most 90% of the nitrogens are quaternary nitrogens, more preferably at most 80% of the nitrogens, further preferably at most 70%, further preferably at most 60%, further preferably at most 50%, even more preferably at most 40%, even more preferably at most 30%, even more preferably at most 20%, even more preferably at most 10%, and most preferably at most 5%. Preferably, the polyelectrolyte has a K_{ow} of >0.4 or ≥0.4, more preferably a K_{ow} of >0.5 or ≥0.5, more preferably >0.5 or ≥0.5, more preferably >0.6 or >0.5, more preferably >0.7 or ≥0.7, more preferably >0.8 or ≥0.8, even more preferably >0.9 or >0.9, still more preferably >1.0 or >1.0, even more preferably >1.5 or >1.5, even more preferably >2.0 or >2.0, even more preferably >2.5 or >2.5 even more preferably >3.0 or >3.0, more preferably >3.5 or >3.5, even more preferably >4.0 or >4.0, even more preferably >5.0 or >5.0, even more preferably >6.0 or ≥, 6.0 even more preferably 7.0 or 27.0, even more preferably 8.0 or 28.0, and most preferably >9.0 or 29.0.

In one embodiment of the present invention, the process for producing a hydrophobic, biodegradable and insulating surface coating for corrosion and/or degradation retardation of solid materials comprises the following steps:

a) providing a solid material,

b) wetting the surface of the solid material under anhydrous conditions with at least one hydrophobic cationic electrolyte or at least one hydrophobic cationic polyelectrolyte or a mixture comprising at least one hydrophobic cationic electrolyte and at least one hydrophobic cationic polyelectrolyte, wherein the hydrophobic electrolyte or cationic polyelectrolyte has a K_{ow}>0.3.

c) drying the surface,

d) wetting of the surface with at least one carboxylic acid under anhydrous conditions,

e) rinsing and drying the surface, and

f) Obtaining the surface coating.

A further embodiment of the present invention directed to a process for producing surface coating comprises the following steps:

a) providing a solid material,

b) wetting the surface of the solid material under anhydrous conditions with at least one hydrophobic cationic electrolyte or at least one hydrophobic cationic polyelectrolyte or a mixture comprising at least one hydrophobic cationic electrolyte and at least one hydrophobic cationic polyelectrolyte, wherein the hydrophobic cationic electrolyte or the hydrophobic cationic polyelectrolyte at least two cationic Carries charge groups or at least two basic groups and is a carbon-containing quaternary nitrogen compound,

c) drying the surface,

d) wetting of the surface with at least one carboxylic acid under anhydrous conditions,

e) rinsing and drying the surface, and

f) Obtaining the surface coating.

One embodiment of the present invention relates to a surface coating comprising or consisting of at least one carboxylic acid; and at least one hydrophobic cationic electrolyte or hydrophobic cationic polyelectrolyte or a mixture comprising at least one hydrophobic cationic electrolyte and at least one hydrophobic cationic polyelectrolyte, wherein the surface coating is prepared under anhydrous conditions, wherein the hydrophobic cationic electrolyte or the hydrophobic cationic polyelectrolyte has at least two cationic charged groups or at least two basic groups and is a carbon-containing quaternary nitrogen compound, wherein the hydrophobic cationic electrolyte or the hydrophobic cationic polyelectrolyte has been rendered hydrophobic by the addition of a carboxylic acid/nitro-carboxylic acid.

Preferably, the at least one hydrophobic cationic electrolyte or hydrophobic cationic polyelectrolyte or a mixture comprising at least one hydrophobic cationic electrolyte and at least one hydrophobic cationic polyelectrolyte is rendered hydrophobic by adding a carboxylic acid/nitro-carboxylic acid.

One embodiment of the present invention relates to a surface coating comprising or consisting of at least one carboxylic acid; and at least one hydrophobic cationic electrolyte or hydrophobic cationic polyelectrolyte or a mixture comprising at least one hydrophobic cationic electrolyte and at least one hydrophobic cationic polyelectrolyte, wherein the surface coating is prepared under anhydrous conditions, wherein the hydrophobic cationic electrolyte or the hydrophobic cationic polyelectrolyte has at least two cationic charged groups or at least two basic groups and is a carbon-containing quaternary nitrogen compound.

These may be hydrocarbon compounds which have an unbranched or branched carbon chain. It is also possible that there is more than one branch. The molecular weight may be between 200 and 500,000 Da, more preferably between 1,000 and 250,000 Da, and more preferably between 5,000 and 125,000 Da. The consistency can be firm to liquid. In the case of solid or highly viscous cationic polyelectrolytes, they can be dissolved in an organic solvent. The cationic groups may be present on one or more nitrogen atoms which are terminal or a link in a compound. It is also possible for 2 or more nitrogen atoms together or even together with other elements to provide positive charge groups.

The hydrophobic cationic electrolyte or the hydrophobic cationic polyelectrolyte is preferably a carbon-containing compound having a molecular weight between 200 and 500,000 Da, which carries at least two cationic charged groups or at least two basic groups which are ionizable and which has a K_{ow} of >0.3.

Another embodiment of the present invention is directed to a process for producing a surface coating comprising the following steps:

a) providing a solid material,

b) wetting the surface of the solid material under anhydrous conditions with at least one hydrophobic cationic electrolyte or at least one hydrophobic cationic polyelectrolyte or a mixture comprising at least one hydrophobic cationic electrolyte and at least one hydrophobic cationic polyelectrolyte, wherein the hydrophobic cationic electrolyte or the hydrophobic cationic polyelectrolyte comprises a carbon-containing compound having a molecular weight of between 200 and 500,000 Da which carries at least two cationic charged groups or at least two basic groups which are ionizable and which has a K_{ow} of >0.3,

c) drying the surface,

d) wetting of the surface with at least one carboxylic acid under anhydrous conditions,

e) rinsing and drying the surface, and

f) Obtaining the surface coating.

Therefore, another embodiment of the present invention relates to a surface coating comprising or consisting of at least one carboxylic acid; and at least one hydrophobic cationic electrolyte or hydrophobic cationic polyelectrolyte or a mixture comprising at least one hydrophobic cationic electrolyte and at least one hydrophobic cationic polyelectrolyte, wherein the surface coating is anhydrously prepared, wherein the hydrophobic cationic electrolyte or the hydrophobic cationic polyelectrolyte comprises a carbon-containing compound having a molecular weight between 200 and 500,000 Da which has at least two cationic charged groups or at least two basic groups which are ionizable and which has a K_{ow} of >0.3.

Nitrogen compounds which provide a cationic charged group or can be converted into cationic charged groups can be, for example, the following groups: amines, amides, ammonium, imines, azanes, triazines, tetrazanes, nitrones, guanidine, amidine, pyrrolidine, piperidine, piperazine, morpholine, N-heteroaromatics, such as imidazole, pyrazole, pyridine, pyrazine, pyrimidine, pyridazine,

isoquinoline, quinoline, benzimidazole, purine, thiazole or oxazole. It is preferred if the cationic charged group consists of a quaternized nitrogen compound. Furthermore the organic polycation has preferably quaternary cationic groups, preferably quaternary amines selected from the group comprising or consisting of ammonium, guanidinium, amidinium, imidazolium, pyrrolidinium, pyrazinium, piperidinium, pyrimidinium, pyridazinium, pyrazolium, isoquinolinium, quinolinium, purinium, benzimidazolium, thiazolinium, oxazolinium, phosphonium and sulfonium ions.

Particular preference is given to quaternized nitrogen atoms or compounds having quaternized nitrogen atoms as cationic charged group carriers.

The heterocyclic aromatics or aliphatic heterocycles may also carry additional amine groups.

Another embodiment of the present invention is directed to a process for producing surface coating comprising the following steps:

a) providing a solid material,

b)) wetting the surface of the solid material under anhydrous conditions with at least one electrolyte or at least one hydrophobic cationic polyelectrolyte or a mixture comprising at least one hydrophobic cationic electrolyte and at least one hydrophobic cationic polyelectrolyte,

c) drying the surface,

d) wetting of the surface with at least one carboxylic acid under anhydrous conditions,

e) rinsing and drying the surface, and

f) obtaining the surface coating, and

wherein the hydrophobic cationic electrolyte or the hydrophobic cationic polyelectrolyte carries at least two cationic charged groups or at least two basic groups selected from the list consisting of or including ammonium, guanidinium, amidinium, imidazolium, pyrrolidinium, pyrazinium, piperidinium, pyrimidinium, pyridazinium, pyrazolium, isoquinolinium, quinolinium, purinium, benzimidazolium, thiazolinium, oxazolinium, phosphonium and sulfonium.

One embodiment of the present invention relates to a surface coating comprising or consisting of at least one carboxylic acid; and at least one hydrophobic cationic electrolyte or hydrophobic cationic polyelectrolyte or a mixture comprising at least one hydrophobic cationic electrolyte and at least one hydrophobic cationic polyelectrolyte, wherein the hydrophobic cationic electrolyte or the hydrophobic cationic polyelectrolyte carries at least two cationic charged groups or at least two basic groups selected from a list comprising or including of ammonium, guanidinium, amidinium, imidazolium, pyrrolidinium, pyrazinium, piperidinium, pyrimidinium, pyridazinium, pyrazolium, isoquinolinium, quinolinium, purinium, benzimidazolium, thiazolinium, oxazolinium, phosphonium and sulfonium.

The hydrophobic cationic electrolytes and polyelectrolytes may also be condensation or polymerization products of hydrophobic carbon compounds and compounds having cationic charged groups or hydrophilic cationic electrolytes which are covalently linked to hydrophobic carbon compounds or covalently bound to each other and to each other by polymerization. Furthermore, they may be hydrophilic cationic polyelectrolytes, which are rendered hydrophobic with hydrophobic compounds (see also the procedures hereafter). It has been found that the cationic groups are very well suited to bind to hydrophobic groups. As a result, the number of charged carriers decreases and the hydrophobicity is increased. It has been found that the saturation of the cationic groups, or the increase of the hydrophobicity, is accompanied by an increase of the degradation stability. This also applies to a saturation of the cationic groups by carboxylic acids which are bound electrostatically. It is believed that cationic groups that do not have hydrophobic shielding promote water ingress/penetration that occurs during degradation. Therefore, cationic electrolytes and polyelectrolytes are preferred in which there is a hydrophobic shielding of the cationic groups by means of electrostatically bound carboxylic acids and/or covalently bound hydrophobic residues, preferably >50%, more preferably >75%, more preferably >90% and most preferably >98% of the cationic groups.

Preference is given to a process in which hydrophobic cationic electrolytes and/or hydrophobic cationic polyelectrolytes are prepared and used for surface coating in which >50% of the cationic groups are replaced by hydrophobic groups.

The hydrophobic cationic polyelectrolytes may comprise amino acids. Suitable are homologous organic polycations, such as polylysine, polyarginine, polyornithine, polyhistidine or heterologous polycations having two or more different amino acids, wherein at least one amino acid can be converted into a positively charged group preferably via a nitrogen unit.

Preferably, the polyamino acids are rendered hydrophobic. In one embodiment of the present invention, an amino acid is copolymerized with an amino acid selected from the group consisting of phenylalanine, lysine, arginine, histidine, ornithine or a mixture of at least two of these amino acids. The said amino acids can be previously rendered hydrophobic or the resulting copolymer is rendered hydrophobic.

Monomeric amino acids can be protected from polymerization. As the protecting group for the functionality to be protected it is preferable to use fluorenylmethoxycarbonyl (Fmoc), tert-butyloxycarbonyl (Boc), benzyloxycarbonyl (Cbz, Z) or acetyl groups, further preferred are triphenylmethyl (Trt), benzyloxymethyl (Bom), benzyl-, phenyl, dinitrophenyl (Dnp), toluenesulfony-I (Tos), mesitylenesulfonyl (Mts), acetamidomethyl (Acm), tert-butylmercapto groups (tBum). Methods are known in the prior art with which the protective groups can be removed again following polymerization. However, the protecting groups can frequently also be reused to produce the desired hydrophobic character of the polyelectrolytes. It has been found that arginine in its protected form already has the desired hydrophobic character of the hydrophobic cationic electrolyte. Particularly preferred is e.g. an arylsulfonyl protecting group. It is also possible via the protecting groups to introduce the desired carbon chain lengths as disclosed herein prior to polymerization. It would also be conceivable to polymerize monomeric amino acid with protective groups having different carbon chain lengths. Alternatively, it would also be possible to only partially deprotect the protected polyamino acid.

In a preferred embodiment, copolymerization with phenylalanine, benzylglutamate or with a mixture comprising or consisting of phenylalanine and benzylglutamate is carried out together with an amino acid selected from the group consisting of lysine, arginine, histidine or a mixture of at least two of these amino acids.

One embodiment of the present invention relates to a surface coating comprising or consisting of at least one carboxylic acid; and at least one hydrophobic cationic electrolyte or hydrophobic cationic polyelectrolyte or a mixture comprising at least one hydrophobic cationic electrolyte and at least one hydrophobic cationic polyelectrolyte, wherein the surface coating is prepared under anhydrous conditions, and wherein the hydrophobic cationic electrolyte or the hydrophobic cationic polyelectrolyte comprises a polymer and/or copolymer or consists of at least one amino acid phenylalanine, lysine, arginine, histidine and/or ornithine.

One embodiment of the present invention relates to a surface coating comprising or consisting of at least one carboxylic acid; and at least one hydrophobic cationic electrolyte or hydrophobic cationic polyelectrolyte or a mixture comprising at least one hydrophobic cationic electrolyte and at least one hydrophobic cationic polyelectrolyte, wherein the surface coating is prepared under anhydrous conditions, and wherein the hydrophobic cationic electrolyte or the hydrophobic cationic polyelectrolyte polyphenylalanine, polylysine, polyarginine, polyornithine, polyhistidine or a compound containing or consisting of the amino acids lysine, arginine, histidine, phenylalanine and/or ornithine.

Therefore, one embodiment of the present invention is directed to a method of making surface coatings comprising the following steps:

a) providing a solid material,

b) wetting the surface of the solid material under anhydrous conditions with at least one hydrophobic cationic electrolyte or at least one hydrophobic cationic polyelectrolyte or a mixture comprising at least one hydrophobic cationic electrolyte and at least one hydrophobic cationic polyelectrolyte, wherein the hydrophobic cationic electrolyte or the hydrophobic cationic polyelectrolyte phenylalanine, polylysine, Polyarginine, polyornithine, polyhistidine or a compound containing or consisting of the amino acids phenylalanine, lysine, arginine, histidine and/or ornithine,

c) drying the surface,

d) wetting of the surface with at least one carboxylic acid under anhydrous conditions,

e) rinsing and drying the surface, and

f) obtaining the surface coating.

Preferably, polymers are obtained by first functionalizing the monomers with a reactive group which preferably reacts with the carboxyl group, such as N-carboxyanhydrides (NCA).

In this case, further functional groups of the compounds which interfere with a polymerization reaction must be protected by known processes.

With these compounds, reaction activation is started/initiated by the addition of a reaction-promoting solvent or a nucleophile such as an amine, e.g. by using a suitable concentration ratio in a solvent phase, such as THF. By “reaction activation” is meant in this context the initiation of the reaction or the initiation of the polymerization. The polymerization reaction can be terminated by known methods. The polymerization products may, if necessary, be fractionated according to their molecular weight. For compounds containing protective groups of cationic functional groups, these should be separated. Thus, if functional groups that can be converted into a cationic group have a protecting group, these are removed in a further step.

If the obtainable hydrophobic cationic polypeptides have the specifications according to the invention, they can be deposited on native or material surfaces that are rendered hydrophobic in a manner analogous to the described surface deposition methods.

State-of-the-art polycationic compounds that do not have the required hydrophobicity can be made by mixing them with non-polar and/or poorly polar compounds. Such methods are known in the art.

In a preferred embodiment, the cationic electrolytes are made hydrophobic according to one of the following methods or according to a method of the prior art.

To achieve a hydrophobic compound, it is preferred that the hydrogen atoms of primary and secondary amino groups be in part replaced by linear or branched alkyl, alkenyl, alkynyl, hydroxyalkyl or alkylcarboxyl radicals having from 6 to 24 carbon atoms, preferably from 8 to 24 carbon atoms, preferably from 10 to 22 carbon atoms, preferably with 12 to 20 carbon atoms, even more preferably with 14 to 18 carbon atoms in the alkyl radical, which may carry further substituents such as alkyl, alkene, alkyne, hydroxy groups, amino groups, halogens, sulfide groups or carboxyl groups.

The reaction is preferably carried out with linear or branched carboxylic acids having 10 to 22 carbon atoms, preferably having 14 to 20 carbon atoms, more preferably 12 to 20 carbon atoms, even more preferably having 14 to 18 carbon atoms in the alkyl or alkylene radical, such as capric acid, undecanoic acid, lauric acid, tridecanoic acid, myristic acid, pentadecanoic acid, palmitic acid, margaric acid, stearic acid, nonadecanoic acid, arachic acid, behenic acid, palmitoleic acid, oleic acid, linoleic acid, linolenic acid, arachidonic acid and their mixtures, preferably stearic acid, palmitic acid and oleic acid, or the acid chlorides, esters or anhydrides of said carboxylic acids and/or linear or branched alkyl halides having 10 to 22 carbon atoms, preferably having 12 to 20 carbon atoms, even more preferably having 14 to 18 carbon atoms, such as tetradecyl chloride, hexadecyl chloride, octadecyl chloride and mixtures thereof and/or

alkylepoxides having 10 to 22 carbon atoms, preferably having 12 to 20 carbon atoms, even more preferably having 14 to 18 carbon atoms, such as hexadecenyl oxide and octadecenyl oxide and mixtures thereof and/or

alkyl chain dimers having 10 to 22 carbon atoms, preferably 12 to 20 carbon atoms, even more preferably 14 to 18 carbon atoms in the alkyl radical, such as lauryl chain, palmityl chain, stearyl chain and oleyl chain dimers and mixtures thereof and/or cyclic dicarboxylic anhydrides, in particular alkyl-substituted succinic anhydrides having 10 to 22 carbon atoms, preferably having 12 to 20 carbon atoms, even more preferably having 14 to 18 carbon atoms in the alkyl radical, such as dodecenylsuccinic, tetradecylsuccinic acid anhydride, hexadecylsuccinic acid anhydride and mixtures thereof and/or alkyl isocyanates having 10 to 22 carbon atoms, preferably 14 to 18 carbon atoms in the alkyl radical, such as tetradecyl isocyanate, hexadecyl isocyanate, octadecyl isocyanate and mixtures thereof and/or chloroformates of fatty alcohols having 10 to 22 carbon atoms, preferably with 12 to 20 carbon atoms, more preferably with 14 to 18 carbon atoms.

In this case, the degree of hydrophobization (the degree of modification) is preferably 0.1 wt % to 100 wt %, more preferably 0.2 wt % to 80 wt %, more preferably 0.3 wt % to 60 wt %, further preferably 0.4 to 50% by weight, more preferably 0.5 to 40% by weight, more preferably 0.6 to 30% by weight, further preferably 0.7 to 20% by weight, further preferably. 9 to 10 wt % and particularly preferably 1 to 7 wt % of the above hydrophobizing units, based on the weight of the finished product.

Preferably, the at least one hydrophobic cationic electrolyte and/or hydrophobic cationic polyelectrolyte is rendered hydrophobic.

Also preferred to make compounds hydrophobic are epoxy groups. In a reaction of primary amino groups with epoxy groups (e.g. epoxyethane, epoxypropane, epoxybutane, epoxypentane, epoxyhexane, epoxyheptane, epoxyoctane, epoxynonane or epoxydecane), the H atom of the amino group is replaced by the carbon of the epoxide building block.

Also preferred are lipophilic polyalkylenepolyamines. These compounds can be obtained according to the prior art, for example by alcohol amination in which aliphatic amino alcohols are reacted with each other or aliphatic diamines or polyamines with aliphatic diols or polyols with elimination of water in the presence of a homogeneous catalyst. At least one of the starting materials has an alkyl or alkylene group containing five or more carbon atoms.

Surprisingly, the hydrophobicity can be selected depending on the hydrophobizing agents used and/or the degree of alkylation of the cationic centers of a cationic electrolyte or polyelectrolyte, whereby a different solution behavior can be achieved in organic solvents that have a different polarity. This can be used in a particularly advantageous manner to allow multiple applications of hydrophobic cationic electrolytes and polyelectrolytes, which are dissolved in organic solvents of different polarities, thereby avoiding the dissolution and/or detachment of an already existing layering of the compounds. This makes it possible, for example, that in the case of prolonged exposure time of a surface which has already been coated according to the invention, the already deposited hydrophobic cationic electrolytes or polyelectrolytes do not dissolve again in the solution. For example, it was possible to coat a material surface with PEI (25 kD/average degree of branching) which already had 80% alkylation with a C-8 alkane and which dissolved well in THF but poorly in pentane, by dip coating in a 5% solution over 30 min. This was followed by drying. Thereafter applying a 5% solution of the same starting compound with 100% alkylation using the same alkylating agent but dissolved in pentane, the application of the mixture by means of a micropipetting method in a defined amount to the material surface was possible. Quantification of the material deposited onto the material surface by determining the weight of the coated material showed that after each coating step a deposition had been achieved and there was a summation of coating layers.

Furthermore, a carboxylic acid/nitro-carboxylic acid, for example, can be applied over an existing coating with a hydrophobic cationic electrolyte or polyelectrolyte, without a relevant dissolution of the previous layer if they are applied in a solution of methanol or acetone (e.g. by spray coating or micropippetting method) or when the material is immersed into an appropriate solution (e.g. by dip-coating). Therefore, a preferred embodiment of the coating according to the invention is the application of hydrophobic cationic electrolytes or polyelectrolytes which have a different hydrophobicity or dissolution behavior in organic solvents, wherein the application of these compounds to a material surface takes place alternately.

In this case, it is preferable to first carry out the deposition of a hydrophobic cationic electrolyte or polyelectrolyte and, after drying, to deposit a layer of a hydrophobic cationic electrolyte or polyelectrolyte onto the material surface which has a lower or higher hydrophobicity and using a different organic solvent. One and/or both of the hydrophobic cationic electrolytes or polyelectrolytes may be present in the form of a mixture with one or more carboxylic acids/nitro-carboxylic acids. According to the invention and also preferred is to carry out the deposition of carboxylic acids/nitro-carboxylic acids, which are preferably dissolved in a polar organic solvent, between the abovementioned coating steps to obtain a separate layer.

The cationic groups of the hydrophobic cationic polyelectrolytes can be present either in protonated form or in quaternized form. Suitable quaternizing agents are alkylating agents such as alkyl halides, methyl chloride, methyl bromide, methyl iodide, ethyl chloride, ethyl bromide, ethyl iodide, propyl chloride, propyl bromide, propyl iodide, butyl chloride, butyl bromide, butyl iodide, pentyl chloride, pentyl bromide, pentyl iodide, hexyl chloride, hexyl bromide, hexyl iodide, heptyl chloride, heptyl bromide, heptyl iodide, octyl chloride, octyl bromide, octyl iodide, nonyl chloride, nonyl bromide, nonyl iodide, decyl chloride, decyl bromide, decyl iodide, undecylchloride, undecylbromide, undecyliodide, dodecyl chloride, dodecyl bromide, dodecyl iodide, tridecylchloride, tridecylbromide, tridecyliodide, tetradecyl chloride, tetradecyl bromide, tetradecyliodide, pentadecylchloride, pentadecylbromide, pentadecyliodide, hexadecyl chloride, hexadecyl bromide, hexadecyl iodide, octadecyl chloride, octadecyl bromide, octadecyl iodide, nonadecyl chloride, nonadecyl bromide, nonadecyl iodide, eicosanyl chloride, eicosanyl bromide, eicosanyl iodide, heneicosanyl chloride, heneicosanyl bromide, heneicosanyl iodide, docosanyl chloride, docosanylbromide, docosanyliodide, tricosanylchloride, tricosanylbromide, tricosanyliodid, tetracosanylchloride, tetracosanylbromide, tetracosanyliodide, pentacosanylchloride, pentacosanylbromide, pentacosanyliodide, hexacosanylchloride, hexacosanylbromide, hexacosanyliodide, heptacosanylchloride, heptacosanylbromide, heptacosanyliodide, octacosanylchloride, octacosanylbromide, octacosanyliodide as well as the mono- or poly-unsaturated derivatives of the aforementioned alkyl halides benzyl chloride, benzyl bromide, or benzyl iodide, alkyl sulfonates of the aforementioned alkanes and their mono- or polyunsaturated forms such as methyl sulfonate or methyl toluene-4-sulfonate, dialkyl sulfates of the aforementioned alkanes and their mono- or polyunsaturated forms such as dimethyl sulfate or diethyl sulfate.

Also preferred are hydrophobic polyalkyleneimines. These are understood as meaning polymers of homologs of ethyleneimine (aziridine) or a 2-substituted-2-oxazoline monomer, such as propylene imine (2-methylaziridine), 1- or 2-butyleneimine (2-ethylaziridine or 2,3-dimethylaziridine).

These may be homopolymers of the abovementioned said alkyleneimines or an oxazoline monomer or are higher homologs and the graft polymers of polyamidoamines or polyvinylamines with ethyleneimine or a 2-substituted-2-oxazoline monomer or are their higher homologs. Polyalkyleneimines may be modified by reaction with alkylene oxides such as ethylene oxide, propylene oxide or butylene oxide, dialkyl carbonates such as dimethyl carbonate and diethyl carbonate, alkylene carbonates such as ethylene carbonate or propylene carbonate, or C₁-C₆ carboxylic acids.

Particularly preferred are hydrophobic polyethyleneimines. These may be homopolymers of ethyleneimine (aziridine) or a 2-substituted 2-oxazoline monomer or its higher homologs, as well as the graft polymers of polyamidoamines or polyvinylamines with ethyleneimine or a 2-substituted 2-oxazoline monomer or its higher homologs. The polyethyleneimines can be uncrosslinked or crosslinked, quaternized and/or modified by reaction with alkylene oxides, dialkyl or alkylene carbonates or C₁- to C₆ carboxylic acids.

In the event that it should be linear polyethyleneamines, the amines are only partially modified with the dialkyl or alkylen carbonates or C1 to C6 carboxylic acids.

For the preparation of the hydrophobic surface, known methods for co/graft polymerization can be used. Examples of compounds with which this can be achieved are: diisocyanates such as hexamethylene diisocyanate, isophorone diisocyanate, dicyclohexylmethane-4,4′-diisocyanate and diphenylmethane diisocyanate, dihaloalkanes such as 1,2-dichloroethane, 1,3-trichloropropane, 1,4-dichlorobutane and 1,6-dichlorohexane, diepoxides such as oligo- and polyethylene glycol bisepoxides, epihalohydrins such as epichlorohydrin, bischlorohydrin ethers of alkylene glycols and polyalkylene glycols having 2 to 100 ethylene oxide and/or propylene oxide units, alkylene carbonates such as ethylene carbonate and propylene carbonate, and bischloroformates such as 2,2-dimethylpropylene bischloroformate.

Furthermore, in the prior art further hydrophobization methods are known, e.g. for polyethyleneimine in WO 2004/087226 or polyvinylamine in WO 97/42229 and WO 03/099880.

Hydrophobic polyethyleneimines can also consist of polymers which have been prepared from ethyleneimine units and polyamidoamines by grafting.

Grafted polyamidoamines are known, for example, from U.S. Pat. No. 4,144,123 or DE-B-2,434,816.

Hydrophobic polyalkylenepolyamines are compounds containing at least 3 basic nitrogen atoms in the molecule, for example diethylenetriamine, dipropylenetriamine, triethylenetetramine, tripropylenetetramine, tetraethylenepentamine, pentaethylenehexamine, N-(2-aminoethyl)-1,3-propanediamine and N, N′-bis (3-aminopropyl) ethylenediamine. These compounds can also be adjusted by copolymerization or grafting to the hydrophobicity of the invention.

Also preferred are hydrophobic polyvinylamines which are homopolymers or copolymers of N-vinylcarboxamides which are at least partially saponified. The polyvinylamines can be present uncrosslinked or crosslinked, quaternized and/or by reaction with alkylene oxides, dialkyl or alkylenecarbonates or be modified C1- to C6-carboxylic acids.

Furthermore, the hydrophobic cationic polyelectrolytes may be compounds prepared from the following monomer subunits: 2-aminoethyl acrylate, 2-aminoethyl methacrylate, dimethylaminoethyl acrylate (DMAEA), dimethylaminopropyl acrylate, propenoic acid 2-(dimethylamino) propyl ester, dimethylaminobutyl acrylate, diethylaminoethyl acrylate, diethylaminopropyl acrylate propenoic acid 2-(diethylamino) propyl ester, diethylaminobutyl acrylate, dipropylaminoethyl acrylate, dipropylaminopropyl acrylate, 2-propenoic acid 2-(dipropylamino) propyl ester, dipropylaminobutyl acrylate, 2-(morpholin-4-yl) ethyl acrylate, 2-(morpholin-4-yl) propyl acrylate propenoic acid 2-(morpholin-4-yl) propyl ester, 2-(morpholin-4-yl) butyl acrylate, 2-(pyrrolidin-1-yl) ethyl acrylate, 2-(pyrrolid-1-yl) propyl acrylate, propenoic acid-2-(pyrrolidin-1-yl) propyl ester, dimethylaminoethyl methacrylate (DMAEM), dimethylaminopropyl methacrylate, 2-(dimethylamino) methacrylate, dimethylaminobutyl methacrylate, diethylamino methyl methacrylate, diethylaminopropyl methacrylate, 2-(diethylamino) propyl methacrylate, diethylaminobutyl methacrylate, dipropylaminoethyl methacrylate, dipropylaminopropyl methacrylate, 2-(dipropylamino) propyl methacrylate, dipropylaminobutyl methacrylate, 2-(morpholin-4-yl) methacrylate, methacrylic acid 2-(morpholine). 4-yl) propyl ester, ethyl 2-(morpholino) methacrylate, methacrylic acid 2-(morpholin-4-yl) butyl ester, methacrylic acid 2-(pyrrolidin-1-yl) ethyl ester, methacrylic acid 2-(pyrrolidin-1) yl) propyl ester, methacrylic acid 2-(pyrrolidin-1-yl) ethyl ester, methacrylic acid 2-(pyrrolidin-1-yl) butyl ester, dimethylaminoethylacrylamide, dimethylaminopropylacrylamide, N [2-(dimethylamino) propyl] acrylamide, dimethylaminobutylacrylamide, diethylaminoethylacrylamide, diethylaminopropylacrylamide, N-[2-(diethylamino) propyl] acrylamide, diethylaminobutylacrylamide, dipropylaminoethylacrylamide, dipropylaminopropylacrylamide, N-[2-(dipropylamino) propyl] acrylamide, dipropylaminobutylacrylamide, N-[2-N-(morpholin-4-yl) ethyl] acrylamide, N-[2-(morpholin-4-yl) propyl] acrylamide, N-[2-(morpholin-4-yl) propyl)] acrylamide, N-[2-(morpholin-4-yl) butyl] acrylamide, N-[2-(pyrrolidin-1-yl) ethyl] acrylamide, N-[2-(pyrrolidin-1-yl) propyl]acrylamide, N-[2-(pyrrolindin-1-yl) propyl)] acrylamide and/or N-[2-(pyrrolidin-1-yl) butyl]acrylamide. The amino units can furthermore be alkylated (made hydrophobic). Also, quaternized amino group can be prepared. Preferred is dimethylaminoethyl acrylate (DMAEA) or dimethylaminoethyl methacrylate (DMAEM) and its quaternized form.

Further, cetylacetyl (imidazol-4-ylmethyl) polyethyleneimine is preferred.

Other compounds which are suitable for the preparation of hydrophobic cationic polyelectrolytes are, for example, compounds with primary, secondary and tertiary amines, such as lower alkylamines, such as methylamine, ethylamine, propylamine, iso-propylamine, butylamine, pentylamine, hexylamine, heptylamine, octylamine, dimethylamine, diethylamine, dipropylamine t-butylamine, procaine, ethanolamine, arylalkylamines, such as dibenzylamine and N, N-dibenzylethylenediamine, heterocyclic amines comprising N-alkylpyrrolidine such as N-methylpyrrolidine, N-ethypyrrolidine, N-propylpyrrolidine, N-alkylpiperidines such as N-methylpiperidine, N-ethylpiperidine, N-propylpiperidine, cycloalkylamines such as cyclopentylamine, N-methylpentylamine, N-ethylcyclopentanamine, dicyclopentylamine, cyclohexylamine, N-methylcyclohexanamine, N-ethylcyclopentanamine, dicyclohexylamine or N-cyclopentylcyclohexanamine.

Particularly preferred are hydrophobic compounds of polyamidoamine (PAMAM), polyethylenimine (PEI) and polypropylenimine (PPI). Particular preference is given to hydrophobized polyamidoamine (PAMAM), hydrophobized polyethylenimine (PEI), hydrophobized polypropylenimine (PPI), and hydrophobized polymers and/or copolymers containing or consisting of arginine.

Particularly preferred are hydrophobic compounds of polyamidoamine (PAMAM), polyethylenimine (PEI) and polypropylenimine (PPI), which are preferably modified as described herein.

In the hydrophobization of the polymeric precursors, thus forming a hydrophobic cationic polyelectrolyte, such as from polyamines, polyethyleneimine (PEI) or polyamino acids, for example, are preferably at least 25%, more preferably at least 30%, even more preferably at least 40% of the aminoprotons of the starting material are replaced by linear or branched alkyl alkenyl, alkynyl, hydroxyalkyl or alkylcarboxyl radicals preferably having at least 6 carbon atoms, preferably having at least 8 carbon atoms, more preferably having at least 10 carbon atoms, even more preferably having at least 12 carbon atoms, even more preferably having at least 14 carbon atoms and even more preferably having at least 16 carbon atoms.

The term “hydrophobization” is preferably understood as meaning the degree of alkylation. In this case, the degree of alkylation is preferably the ratio of the alkylating reagent used to the number of primary amines and/or secondary and/or tertiary amines, more preferably the ratio of the alkylating reagent used to the number of primary amines and/or secondary and most preferably the ratio of the alkylating reagent used number of primary amines. The degree of alkylation is particularly preferably 1-100%, preferably 10-100%, more preferably 20-100%, more preferably 30-100%, more preferably 40-100%, still more preferably 50-100%, even more preferably from 60 to 100%, more preferably from 70 to 100%, even more preferably from 80 to 100%, and still more preferably from 90 to 100%.

The at least one polyelectrolyte is particularly preferably selected from the group consisting of polybenzylamine, polyvinylpyrrolidine, polyvinylpiperidine, polyvinylimidazole, polyvinylpyridine, polyvinylamine, polyvinylguanidine, polyvinylamidine, polyallylamine, polyallylguanidine, diallyl, polydiallyldimethylammonium salt (PDADMA salt), hexadimethrine bromide (polybrene), poly [bis (2-chloroethyl) ether-alt-1,3-bis [3-(dimethylamino) propyl] urea] and copolymers of the aforementioned polymers, wherein the polyelectrolytes are preferably hydrophobicized.

### Active and Supportive Compounds

The terms “active compound” and “supportive compound” mean molecular compounds which have a known effect in biological systems or can cause an effect on biological systems.

The active compounds meant herein are preferably molecular compounds known as medicinal agents. The preferred supportive compounds are, for example, those compounds which regulate a release of the active compounds and/or have an influence on their stability or exert an influence on the degradation of the coating and/or have biological effects.

In principle, all compounds that can be used and formulated or combined with one of the hydrophobic cationic polyelectrolytes according to the invention can be use.

A formulation can also be carried out with a carboxylic acid and a suitable substance, such as an active ingredient or a supportive compound, admixed to one of the hydrophobic cationic polyelectrolytes or can be deposited on a coating.

However, such compounds can also be electrostatically bound to hydrophobic anionic electrolytes/polyelectrolytes and deposited together with them on a layer of a hydrophobic cationic polyelectrolyte or deposited together with them during the deposition process.

The active compound is preferably an antiproliferative, antiinflammatory, antimigrative, antiphlogistic, antiangiogenic, cytostatic, cytotoxic, antirestenotic, antineoplastic, antibacterial and/or antimycotic active ingredient.

As antiproliferative, anti-inflammatory, antimigrative, anti-phlogistic, anti-angiogenic, cytostatic, cytotoxic, antirestenotic, antineoplastic, antibacterial and/or antifungal agents are meant e. g. abciximab, acemetacin, acetylvismion B, aclarubicin, ademetionin, adriamycin, aescin, afromosone, akagerin, aldesleukin which are preferably used, further amidorone, aminoglutethemide, amsacrine, anakinra, anastrozole, anemonin, anopterin, antimycotics, antithrombotics, apocymarin, argatroban, aristolactam-All, aristolochic acid, ascomycin, asparaginase, aspirin, atorvastatin, auranofin, azathioprine, azithromycin, baccatin, bafilomycin, basiliximab, bendamustine, benzocaine, berberine, betulin, betulinic acid, bilobol, bisparthenolidine, bleomycin, bombrestatin, boswellic acids and their derivatives, bruceanols A, B and C, bryophyllin A, busulfan, antithrombin, bivalirudin, cadherins, camptothecin, capecitabine, o-carbamoylphenoxyacetic acid, carboplatin, carmustine, celecoxib, cepharantin, cerivastatin, CETP inhibitors, chlorambucil, chloroquine phosphate, cictoxin, ciprofloxacin, cisplatin, cladribine, clarithromycin, colchicine, concanamycin, coumadin, C-type natriuretic peptides (CNP), cudraisoflavone A, curcumin, cyclophosphamide, cyclosporine A, cytarabine, dacarbazine, daclizumab, dactinomycin, dapsone, daunorubicin, diclofenac, 1,11-dimethoxycanthin-6-one, docetaxel, doxorubicin, dunaimycin, epirubicin, epothilones A and B, erythromycin, estramustine, etoboside, everolimus, filgrastim, fluroblastin, fluvastatin, fludarabine, fludarabine 5′-dihydrogen phosphate, fluorouracil, folimycin, fosfestrol, gemcitabine, ghalacinoside, ginkgol, ginkgolic acid, glycoside 1a, 4-hydroxyoxycyclophosphamide, idarubicin, ifosfamide, josamycin, lapachol, lomustine, lovastatin, melphalan, midecamycin, mitoxantrone, nimustin, pitavastatin, pravastatin, procarbazine, mitomycin, methotrexate, mercaptopurine, thioguanine, oxaliplatin, irinotecan, topotecan, hydroxycarb amid, miltefosine, pentostatin, pegasparase, exemestane, letrozole, formestan, mitoxanthrone, mycophenolate mofetil, β-lapachone, podophyllotoxin, podophyllic acid 2-ethylhydrazide, molgramostim (rhuGM-CSF), peginterferon α-2b, lanograstim (r-HuG-CSF), macrogol, selectin (cytokine antagonist), cytokine inhibitors, COX-2 inhibitor, angiopeptin, monoclonal antibodies that inhibit muscle cell proliferation, bFGF antagonists, probucol, prostaglandins, 1-hydroxy-11-methoxycanthin-6-one, scopolectin, NO Donors, pentaerythritol tetranitrate and syndnoeimines, S-nitrosated derivatives, tamoxifen, staurosporine, β-estradiol, α-estradiol, estriol, estrone, ethinyl estradiol, medroxyprogesterone, estradiol cypionates, estradiol benzoates, tranilast, camebakaurin and other terpenoids used in cancer therapy, verapamil, tyrosine kinase inhibitors (tyrphostins), paclitaxel and its derivatives, 6-a-hydroxy paclitaxel, taxotere, mofebutazone, lonazolac, lidocaine, ketoprofen, mefenamic acid, piroxicam, meloxicam, penicillamine, hydroxychloroquine, sodium aurothiomalate, oxaceprol, β-sitosterol, myrtainaine, polidocanol, nonivamide, levomenthol, ellipticin, D-24851 (Calbiochem), colcemid, cytochalasin AE, indanocine, nocadazole, bacitracin, vitronectin receptor antagonists, azelastine, guanidyl cyclase metal protease 1 and 2 tissue inhibitor stimulator, free nucleic acids, nucleic acids incorporated into virus carriers, DNA and RNA fragments, plaminogen activator inhibitor-1, plasminogen activator inhibitor-2, antisense oligonucleotides, VEGF inhibitors, IGF-1, active ingredients from the group of antibiotics, cefadroxil, cefazolin, cefaclor, cefotixine tobramycin, gentamycin, penicillins, dicloxacillin, oxacillin, sulfonamides, metronidazole, enoxoparin, heparin, hirudin, PPACK, protamine, prourokinase, streptokinase, warfarin, urokinase, vasodilators, dipyramidol, trapidil, nitroprusside, PDGF antagonists, triazolopyrimidine, seramin, ACE inhibitors, captopril, cilazapril, lisinopril.

Enalapril, losartan, thioprotease inhibitors, prostacyclin, vapiprost, interferon α, β and γ, histamine antagonists, serotonin blockers, apoptosis inhibitors, apoptosis regulators, halofuginone, nifedipine, paracetamol, dexpanthenol, clopidogrel, acetylsalicylic acid derivatives, streptomycin, neomycin, framycetin, paromomycin, ribostamycin, kanamycin, amikacin, arbekacin, bekanamycin, dibekacin, spectinomycin, hygromycin B, paromomycin sulfate, netilmicin, sisomicin, isepamicin, verdamicin, astromicin, apramycin, geneticin, amoxicillin, ampicillin, bacampicillin, pivmecillinam, flucloxacillin, mezlocillin, piperacillin, azlocillin, temocillin, ticarcillin, amoxicillin, clavulanic acid, ampicillin, sulbactam, piperacillin, tazobactam, sulbactam, cefamandole, cefotiam, cefuroxime, cefmenoxime, cefodizime, cefoperazone, cefotaxime, ceftazidime, cefsulodin, ceftriaxone, cefepime, cefpirome, cefoxitin, cefotetan, cefalexin, cefuroxime axetil, cefixime, cefpodoxime, ceftibuten, Imipenem, Meropenem, Ertapenem, doripenem, aztreonam, spiramycin, azithromycin, telithromycin, quinopristin, dalfopristin, clindamycin, tetracycline, doxycycline, minocycline, trimethoprim, sulfamethoxazole, sulfametrole, nitrofurantoin, lomefloxacin, norfloxacin, ciprofloxacin, ofloxacin, fleroxacin, levofloxacin, sparfloxacin, moxifloxacin, vancomycin, teicoplanin, linezolid, daptomycin, rifampicin, fusidic acid, fosfomycin, trometamol, chloramphenicol, metronidazole, colistin, mupirocin, bacitracin, neomycin, fluconazole, itraconazole, voriconazole, posaconazole, amphotericin B, 5-flucytosine, caspofungin, anidulafungin, tocopherol tranilast, molsidomine, tea polyphenols, epicatechingallate, epigallocatechin gallate, leflunomide, etanercept, sulfasalazine, etoposide, dicloxacylline, tetracycline, triamcinolone, mutamycin, procainimide, retinoic acid, quinidine, disopyrimide, flecainide, propafenone, sotolol, natural and synthetic steroids, inotodiol, maquiroside A, ghalakinoside, mansonine, strebloside, hydrocortisone, betamet hason, dexamethasone, nonsteroidal substances (NSAIDS), fenoporfen, ibuprofen, indomethacin, naproxen, phenylbutazone, antiviral agents, acyclovir, ganciclovir, zidovudine, clotrimazole, flucytosine, griseofulvin, ketoconazole, miconazole, nystatin, terbinafine, antiprozoal agents, chloroquine, mefloquine, Quinine, natural terpenoids, hippocaesculin, barringtogenol C21-angelate, 14-dehydroagrostistachin, agroscerin, agrostistachin, 17-hydroxyagrostistachin, ovatodiolide, 4,7-oxycycloanisomelic acid, baccharinoids B1, B2, B3 and B7, tubeimoside, bruceantinoside C, yadanzioside N, and P, isodeoxyelephantopin, tomenphantopin A and B, coronary A, B, C and D, ursolic acid, hyptate acid A, iso-iridogermanal. Maytenfoliol, effusantin A, excisanin A and B, longikaurin B, sculponeatin C, kamebaunin, leukamenin A and B, 13,18-dehydro-6-alpha-senecioyloxychaparrine, taxamairin A and B, regenilol, triptolide, cymarin, hydroxyanopterin, protoanemonin, cheliburine chloride, sinococulin A and B, dihydronitidine, nitidinium chloride, 12-beta-hydroxypregnadiene 3,20-dione, helenaline, indicin, indicin N-oxide, lasiocarpine, inotodiol, podophyllotoxin, justicidin A and B, larreatin, malloterine, mallotochromanol, isobutyrylmallotochromanol, maquiroside A, marchantin A, maytansin, lycoridicin, margetin, pancratistatin, liriodenin, bispsrthenolidine, oxoushinsunin, periplocoside A, ursolic acid, deoxypsorospermin, psycorubin, ricin A, sanguinarine, manuwuic acid, methylsorbifolin, sphatheliachromen, stizophyllin, mansonin, streblosid, dihydrousambaraensin, hydroxyusambarin, strychnopentamin, strychnophyllin, usambarin, usambarensin, liriodenin, oxoushinsunin, daphnoretin, lariciresinol, methoxylariciresinol, syringaresinol, sirolimus (rapamycin) and its derivatives such as biolimus A9, everolimus, myolimus, novolimus, pimecrolimus, ridaforolimus, deoxorapamycin, tacrolimus FK 506, temsirolimus and zotarolimus, somatostatin, tacrolimus, roxithromycin, troleandomycin, simvastatin, rosuvastatin, vinblastine, vincristine, vindesine, teniposide, vinorelbine, tropfosfamide, treosulfan, T remozolomide, thiotepa, tretinoin, spiramycin, umbelliferone, desacetylvismion A, vismion A and B, zeorin, and sulfur-containing amino acids such as cystine and salts, hydrates, solvates, enantiomers, racemates, enantiomer mixtures, diastereomer mixtures; metabolites, prodrugs and mixtures of the aforementioned active ingredients.

Preferred active compounds are, for example, heparin, aspirin, ramipril, trapidil, batroxobin, corticoids, such as dexamethasone or 17-beta-estradiol, m-TOR inhibitors, such as rapamycin (sirolimus, everolimus, tarcolimus, zotarolimus, biolimus), taxols, such as paclitaxel.

Preferred supportive compounds are, for example, cholesterol, angiopeptin, VEGF, PEG, collagen, hyaluronic acid and its derivatives, antioxidants, vitamins, such as calciferol or carotenoids. Also, nitro-fatty acids as described herein. Further, phospholipids bearing nitro-fatty acids as described herein as an alkyl substituent. One or both alkyl chains may be substituted by a nitro-fatty acid.

### Carboxylic Acids

Carboxylic acids are organic compounds that carry one or more carboxyl groups. A distinction is made between aliphatic, aromatic and heterocyclic carboxylic acids. Aliphatic forms of carboxylic acids, also called alkanoic acids, are fatty acids and are further listed in the following paragraph.

### Fatty Acids

In general, fatty acids are aliphatic carbon chains having a carboxylic acid group. The carbon atoms may be linked with single bonds (saturated fatty acids) or with double bonds (unsaturated fatty acids), these double bonds may be in a cis or trans configuration. As defined herein, as fatty acids, such compounds having more than 4 consecutive carbon atoms adjacent to the carboxyl group are referred to as fatty acids. Examples of linear saturated fatty acids are nonanecarboxylic acid (capric acid), dodecanoic acid (lauric acid), tetradecanoic acid (myristic acid), hexadecanoic acid (palmitic acid), octadecanoic acid (stearic acid), n-eicosanoic acid (arachic acid) and n-docosanoic acid (behenic acid).

Examples of mono-olefin fatty acids are myristoleic acid, palmetoleic acid, petroselinic acid, oleic acid, elaidic acid, dicelic acid and erucic acid.

Examples of polyolefin fatty acids are linoleic acid, linolenic acid, punicic acid, arachidonic acid and nervonic acid.

Fatty acids may also carry functional groups, such as vernolic acid, ricinoleic acid and lactobacillic acid. The functional groups herein also include terminal carbon cyclic residues.

Examples of the term “fatty acids” used herein include, for example, hexanoic acid, octanoic acid, decanoic acid, dodecanoic acid, tetradecanoic acid, hexadecanoic acid, heptadecanoic acid, octadecanoic acid, eicosanoic acid, docosanoic acid, tetracosanoic acid, cis-9-tetradecenoic acid, cis-9-hexadecenoic acid, cis-6-octadecenoic acid, cis-9-octadecenoic acid, cis-11-octadecenoic acid, cis-9-eicosenoic acid, cis-11-eicosenoic acid, cis-13-docosenoic acid, cis-15-tetracenoic acid, t9-octadecenoic acid, t11-octadecenoic acid, t3-hexadecenoic acid, 9,12-octadecadienoic acid, 6,9,12-octadecatrienoic acid, 8,11,14-eicosatrienoic acid, 5,8,11,14-eicosatetraenoic acid, 7,10,13,16-docosatetraenoic acid, 4,7,10,13,16-docosapentaenoic acid, 9,12,15-octadecatrienoic acid, 6,9,12,15-octadecatetraenoic acid, 8,11,14,17-eicosatetraenoic acid, 5,8,11,14,17-eicosapentaenoic acid, 7,10,13,16,19-docosapentaenoic acid, 4,7,10,13,16,19-docosahexaenoic acid, 5,8,11-eicosatrienoic acid, 9c11t13t-eleostearic acid, 8t10t12c-calendulic acid, 9c11t13c-catalpinic acid, 4,7,9,11,13,16,19-docosaheptadecanoic acid, taxoleinic acid, pinolenic acid, sciadic acid, 6-octadecanoic acid, t11-octadecene-9-amino acid, 9-octadecanoic acid, 6-octadecene 9-amino acid, t10-heptadecene-8-amino acid, 9-octadecene-12-acetic acid, t7, t1l-octadecadiene-9-amino acid, t8, t10-octadecadien-12-acetic acid, 5,8,11,14-eicosatetraic acid, retinoic acid, isopalmitic acid, pristanoic acid, phytanic acid, 11,12-methylene-octadecanoic acid, 9,10-methylene-hexadecanoic acid, coronaric acid, (R, S)-liponic acid, (S)-liponic acid, (R)-lipoic acid, 6,8 (methylsulfanyl) octanoic acid, 4,6-bis (methylsulfanyl) hexanoic acid, 2,4-bis (methylsulfanyl) butanoic acid, 1,2-dithiolane carboxylic acid, (R, S)-6,8-dithiane octanoic acid, (S)-6,8-dithiane octanoic acid, tariric acid, santalbic acid, stearolic acid, 6,9-octadecenoic acid, pyrulic acid, crepenoic acid, heisteric acid, t8, t10 octadecadiene 12-acid, ETYA, cerebronic acid, hydroxynvonic acid, ricinoleic acid, lesquerolic acid, B rassylic acid, and thapsic acid, phytic acid, sinapinic acid, cinnamic acid, and trihydroxybenzoic acid.

### Nitrocarboxylic Acids

The nitrocarboxylic acids which can be used according to the invention, which are also referred to herein synonymously as nitro-fatty acids or “nitrated fatty acids”, contain at least one carboxylic acid unit and one C—NO₂ unit (bond between carbon and nitrogen), and preferably correspond to the general formula X:

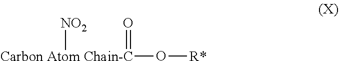

In the formula (X), the residue R * is hydrogen.

As indicated in general formula (X), the compound contains at least one nitro (—NO₂) group attached to one of the carbon atoms of the carbon chain. The nitro group represented by the general formula (X) has no specific position; it may be attached to any one of the carbon atoms (a bis (o) of the alkyl chain, i.e., being bound to any one of the carbon atoms of the carbon atom chain. Most preferred is when the nitro group or groups are/is attached to a vinyl unit of the unsaturated alkyl chain of an unsaturated carboxylic acid. Thus, the nitro group(s) is most preferably attached to a double bond in the unsaturated alkyl chain of the unsaturated carboxylic acid. However, it is possible that the carbon atom chain, which may be referred to as an alkyl chain, may contain more than one nitro group. In addition, the carbon atom chain may also contain double bonds and/or triple bonds and may be linear or branched and contain further substituents defined as substituents S1 to S20. Thus, the term “alkyl chain” not only refers to linear and saturated alkyl groups, but also refers to mono-unsaturated, polyunsaturated, branched and further substituted alkyl groups or alkenyl groups or alkynyl groups. Preferred are the mono-, di- and poly-unsaturated carbon atom chains of the unsaturated carboxylic acids (including unsaturated carboxylic acid esters). Most preferred are double bonds in the carbon atom chain of the carboxylic acid, while triple bonds and saturated carbon atom chains of the unsaturated carboxylic acid are less preferred.

Thus, the carbon atom chain refers to an alkyl chain to which at least one nitro group consisting of 1 to 40 carbon atoms is bonded, in which the alkyl chain may contain one or more double and/or one or more triple bonds and may be cyclic and/or may be substituted by one or more nitro groups and/or one or more substituents S1-S20. When the term “alkyl” is unclear, due to the fact that an alkyl group is saturated and contains no double or triple bonds, the following definition is provided: the term “carbon atom chain” refers to an alkyl chain or alkenyl chain or alkynyl chain to which at least one nitro group consisting of 1 to 40 carbon atoms is bonded, whereby the alkyl chain may be cyclic and substituted by one or more nitro groups and/or one or more substituents S1-S20, the alkenyl chain may contain one or more double bonds and may be cyclic and substituted by one or more nitro groups and/or one or more substituents S1-S20 and the alkynyl chain may contain one or more triple bonds and may be cyclic and by one or more nitro groups and/or one or more substituents S1-S20. The term “may be substituted by one or more nitro groups” is understood to mean that one or more nitro groups may be present on the carbon atom chain in addition to the one nitro group necessarily required and explicitly mentioned and designated in the general formula (X). The term “carbon atom chain” refers to an alkyl chain which is saturated or which may contain one or more double bonds and/or triple bonds or refers to an alkyl chain (only saturated carbon atom chains are meant), alkenyl chain or alkynyl chain having at least one nitro group which is the nitro group that is explicitly drawn and mentioned in the general formula (X). The carbon atom chain preferably contains 1 to 10, more preferably 1 to 5 double bonds or vinyl radicals. The carbon atom chain consists of 1 to 40 carbon atoms, preferably 2 to 30 carbon atoms and more preferably 4 to 24 carbon atoms, where this alkyl chain may contain one or more double and/or one or more triple bonds and/or may be substituted by one or more nitro groups and/or one or more substituents S1-S20.

According to the invention, a preferred subgroup of nitro-carboxylic acids are unsaturated nitro-carboxylic acids. According to the invention, it is possible to use both cis and trans isomers and (depending on the substituents which can produce chiral centers) enantiomers, diastereomers and their racemates. The nitro group can be attached to any position of the carbon chain.

Preferred unsaturated nitro-carboxylic acids are: nitro-cis-9-tetradecenoic acid (nitro-myristoleic acid), nitro-cis-9-hexadecenoic acid (nitro-palmitoleic acid), nitro-cis-6-hexadecenoic acid (nitro-salpenoic acid), nitro-cis-6 (nitro-petrosulic acid), nitro-cis-9-octadecenoic acid (nitro-oleic acid), nitro-trans-9-octadecenoic acid, trans-9-nitro-9-octadecenoic acid, trans-10-nitro-9-octadecenoic acid, cis-9-nitro-9-octadecenoic acid, cis-10 nitro-9-octadecenoyl, trans-10-nitro-8-octadecenoic acid, trans-9-nitro-10-octadecenoic acid, cis-10-nitro-8-octadecenoic acid, cis-9-nitro-10-octadecenoic acid, 9,10-dinitro-octadecenoic acid, nitro-cis-11-octadecenoic acid (nitro-vaccinic acid), nitro-cis-9-eicosenoic acid (nitro-gadolinic acid), nitro-cis-11-eicosenoic acid (nitro-goic acid), nitro-cis-13-docosenoic acid (nitro-erucinic acid), nitro cis-15-tetracenoic acid (nitro-nevonic acid), nitro-t9-octadecenoic acid (nitro-elaidic acid), nitro-t11 (nitro-t-vaccenic acid), nitro-t3-hexadecenoic acid, nitro-3,12-octadecadienoic acid (nitro linoleic acid), nitro-6,9,12-octadecatrienoic acid (nitro-γ-linolenic acid), nitro-8,11,14-eicosatrienoic acid (nitro-dihomo-γ-linolenic acid), nitro-5,8,11,14-eicosatrienoic acid (nitro-arachidonic acid), Nitro-7,10,13,16-docosatetraenoic acid, nitro-4,7,10,13,16-docosapentaenoic acid, nitro-9,12,15-octadecatrienoic acid (nitro-ol-linolenic acid), nitro-6,9,12,15-octadecatetraenoic acid (nitro-stearidonic acid), nitro-8,11,14,17-eicosatetraenoic acid, nitro-5,8,11,14,17-eicosapentaenoic acid (nitro-EPA), nitro-7,10,13,16,19-docosapentaenoic acid (nitro-DPA), nitro-4,7,10,13,16,19-docosahexaenoic acid (nitro-DHA), nitro-5,8,11-eicosatrienoic acid (nitro-madic acid), nitro-9c11t13t-eleostearic acid, Nitro-8t-10t-12c-calendinic acid (nitro-stellaheptanoic acid), nitro-taxolic acid, nitro-pinolenic acid, nitro-sciadoic acid, nitro-6-octadecanoic acid (nitro-taric acid), nitro-saxinic acid, nitro-saxinic acid, nitro-pinolic acid, nitro-acetic acid. Particularly preferred are the nitro-fatty acids of oleic acid. Particular preference is given to nitro-cis-9-octadecenoic acid, nitro-trans-9-octadecenoic acid or a mixture of these or trans-9-nitro-9-octadecenoic acid, cis-9-nitro-9-octadecenoic acid, cis-9-nitro-10-octadecenoic acid, and trans-9-nitro-10-octadecenoic acid or mixtures of these.

Examples of nitro-carboxylic acids with saturated alkyl chains are: nitro-carboxylic acid (nitro-capric acid), nitro-decanoic acid (nitro-capric acid), nitro-decanoic acid (nitro-myrinic acid), nitro-tetradecanoic acid (nitro-myrotic acid), nitro-hexadecenoic acid (nitro-palmitic acid), nitro-heptadecanoic acid (nitro-margaric acid), nitro-octadecanoic acid (nitro-stearic acid), nitroic acid (nitro-arahoic acid), nitro-tocanoic acid (nitro-succinic acid), nitro tetracosan acid (nitro lignocer acid). These and other saturated nitro-carboxylic acids may contain 1, 2, 3, 4, 5 or 6 additional nitro groups and may contain one or more of the above-mentioned substituents S1-S20.

However, unsaturated nitro-carboxylic acids are preferred and, furthermore, unsaturated nitro-carboxylic acids containing one or two nitro groups are preferred.

The concentration of the at least one nitro-carboxylic acid and other active ingredients, if present, is preferably in the range of 0.001-500 mg per cm², preferably 0.01-500 mg per cm 2, preferably 0.01-500 mg per cm 2, more preferably 1-500 mg/cm 2, more preferably 0.001-450 mg per cm 2, more preferably 0.01-450 mg per cm 2, more preferably 0.1-450 mg per cm 2, more preferably 1-450 mg per cm 2, more preferably 0.001-400 mg per cm 2, more preferably 0.01-400 mg per cm 2, more preferably 0.1-400 mg per cm 2, more preferably 1-400 mg per cm 2, more preferably 0.001-300 mg per cm 2, more preferably 0.01 300 mg per cm 2, more preferably 0.1-300 mg per cm 2, more preferably 1,300 mg per cm 2, more preferably 0.001-200 mg per cm 2, more preferably 0.01-200 mg per cm 2, more preferably 0.1-200 mg per cm 2, more preferably 1-200 mg per cm 2, and still more preferably 10-100 mg per cm 2 of the fully coated surface of the medical device preferably endo-prosthesis, where the surface is calculated based on the total surface area.

### Methods

### Method for Carrying Out Step a):

The object of this method step is to provide a material surface in a form in which it can be coated with the method according to the invention. In principle, any material surface can be used for this purpose. For example, the material may be metal or a metal alloy, a plastic, an inorganic compound such as ceramic or porcelain or an enamel, or a synthetic polymer such as PLA or a biopolymer such as cellulose. These can range from resistant to completely dissolvable materials. The material can be closed or open-pored or textured, with a smooth to rough surface. Composites are also suitable. The surfaces may be absorbent or nonimpregnable. The surface properties may vary between hydrophilic oleophobic to lipophilic oleophilic. Preference is given to metals and metal alloys and polymers. Particularly preferred are materials that are easily corrodible and/or biodegradable. Also preferred are smooth surfaces that are hydrophobic and/or lipophilic.

Preferred are surfaces that are completely free of ablatable buildup, erosion/corrosion products such as hydroxides, and are free of osmotically active compounds such as salts or sugar compounds.

Preference is therefore given to methods for surface preparation, such as electropolishing, an ultrasonic cleaning bath or plasma treatment. Preference is given to intensive material surface cleaning with non-corrosive aqueous and/or non-aqueous cleaning agents or purification steps, such as, for example, H₂O₂, acetone, alcohols, dichloromethane. A surface passivation is preferred. This can be done with prior art techniques, such as heating a material consisting of, for example, a metal, silicon, or alkaline earth metal. Preference is given to minimizing superficial OH groups. Preference is given to an interlayer-free material surface. Preferred cleaning agents are, for example, organic solvents, such as alcohols, acetone or chloroform, furthermore acids, such as, for example, H₂SO₄, and also alkalis, such as NaOH, or oxidizing agents, such as H₂O₂. The duration of the exposure and the cleaning process are material-dependent and can be determined by a person skilled in the art. The cleaning result can be checked by analytical methods such as electron microscopy.

### Method for Carrying Out Step a1)

If the material surface does not already have hydrophobic surface properties, the material surface must be rendered hydrophobic in the optional process step a1). The term “hydrophobing” (hydrophobization) is known to those skilled in the art and generally refers to altering the interaction of a substance or material with water, thereby lowering the affinity of the substance or material for water.

The hydrophobicity of an interface can preferably be indicated by its water contact angle. Hydrophobization of the surface of the material is preferably carried out until a water contact angle of >40°, preferably of >60°, more preferably of >70°, and most preferably >85°.

For this purpose, methods are known from the prior art. These are preferably to be used when the surface to be coated has hydrophilic surface properties, which means having a water contact angle of <40°. In the case of the hydrophobization processes, it is possible to distinguish those which permit covalent bonding of hydrophobic compounds and those in which an electrostatic attachment of hydrophobic compounds takes place. A surface hydrophobization according to the invention is achieved when the water contact angle is >60°. According to the invention, the hydrophobization occurs in that the surfaces do not come into contact with water. A covalent surface can be rendered hydrophobic, for example, by silicon compounds which, for example, carry alkane or alkyl compounds and can condense with OH groups.

This can be performed in an anhydrous manner from a solvent phase, for example using t-butanol or diacetone alcohol.

Preferred are organosilanes such as n-octadecyltrimethoxysilane C₂₁H₅₂O₃S (ODTMS) or n-octadecyltriethoxysilane C₂₄H₄₆O₃Si (ODTES), and bivalent silanes such as bis[3-(triethoxysilyl)propyl] tetrasulfide (BTES) and bis(trimethoxysilylpropyl) or aminosilanes such as 3-aminopropyltriethoxysilane (APTES), but also halogenated silanes, such as perfluorodecyltrichlorosilanes.

To prepare the silanization solution, for example, 2-10% of the silane with 5% triethylamine is added to dried toluene. The incubation of the samples takes place, for example, for 2.5 hours in pre-silanized glass vessels at 75° C. The vessels are kept in motion with a sample shaker at a low number of revolutions. After incubation, the samples are removed and rinsed several times with toluene to remove only weakly adhering silane molecules. Thereafter, the samples are dried with compressed air or in a stream of nitrogen and stored for 1.5 hours at 135° C. in an oven.

For the adsorptive application of a hydrophobic agent, compounds are preferred which have an affinity for OH groups. Further preferred are compounds that are metabolized and/or excreted by the human organism and have low toxicity. Dopamine and polydopamine are preferred. The application may be carried out under anhydrous conditions, e.g. with the compounds solubilized in methanol.

Further, other methods of hydrophobing are available, such as a coating with parylene. This can be parylene C, D, N or F, which is preferably applied over the entire surface by means of a gas deposition process.

Another possibility of hydrophobing consists of a coating with halogenated organic or inorganic compounds, wherein the preferred halogens are fluorine, chlorine and bromine.

Examples are PTFE or chlorinated paraffins. Such coatings can also be deposited via a gas phase or applied in the form of a melt.

The compounds for hydrophobization are preferably applied under inert gas conditions and at suitable temperatures adapted to the material to be coated. Deposition of hydrophobing compounds can also be accomplished by physicochemical deposition techniques. Known methods are ALD, PVD or CVD. Preferably, the processes for hydrophobization are carried out by monolayer coatings.

The successful hydrophobing can be determined by known analytical methods, such as the water contact angle measurement. Preference is given to a hydrophobing which results in a water contact angle of >60°, more preferably of >70° and particularly preferably of >85^{°}

### Method for Carrying Out Step b)

The hydrophobic cationic electrolytes and polyelectrolytes of the invention are carbon-based compounds having one or more positive charge groups. Preferred are compounds having a molecular weight between 200 and 500,000 Da, more preferred are those having a molecular weight between 1,000 and 100,000 Da, and more preferably between 2,000 and 50,000 Da. According to the invention, both unbranched and singly or multiply branched hydrophobic cationic electrolytes and polyelectrolytes can be used. Branched and multi-branched hydrophobic cationic polyelectrolytes are preferred. Mixtures of hydrophobic cationic electrolytes and polyelectrolytes with different C-chain length and/or degree of branching can also be used. A hydrophobic property of the hydrophobic cationic electrolytes and polyelectrolytes to be used according to the invention is preferred. This can be recognized, for example, from the fact that after application to a surface, the hydrophobic cationic electrolytes or polyelectrolytes can not be redissolved with an aqueous medium. Furthermore, they impart hydrophobic properties after drying the material surface, which can be determined by means of water contact angle measurements. The hydrophobicity produced thereby causes a water contact angle when measured at 20° C. of preferably >70° or >70°, more preferably >80° or >80°, more preferably >90° or >90°, more preferably >100° or >100° and even more preferably >110° or >110°, and most preferably >120° or 120°. Preferred hydrophobic cationic electrolytes and polyelectrolytes have a K_{ow} of >0.3, more preferably of >0.8, and even more preferably of >1. Hydrophobization does not mean that the hydrophobic cationic electrolytes and polyelectrolytes cannot be partially soluble in an aqueous medium.

For application of the hydrophobic cationic electrolytes and polyelectrolytes, their distribution in a mixed solution is preferred. The mixed solution may consist of one or more organic solvents. Preferred solvents are: volatile alcohols, such as ethanol, methanol, isopropyl alcohol, but also low-volatile alcohols, such as polyethylene glycol or wax alcohols or polyethers, furthermore chlorinated hydrocarbons, such as dichloromethane, volatile alkane, alkene or alkyne compounds in aliphatic or cyclic form, such as heptane or cyclohexane or benzenes.

Further, apolar organic solvents such as toluene or THF. In principle, all nonpolar solvents are suitable. The following may be used as solvents: The following alkanes including their constitutional isomers and their cyclic form pentane, hexane, heptane, octane, nonane, decane, undecane, or dodecane, benzene, toluene, xylenes, trimethylbenzene, ethers, halogenated solvents or alcohols. Specifically, it may be the following solvent treatments: cyclopentane, n-pentane, cyclohexane, n-hexane, 2-methylpentane, 3-methylpentane, 2,2-dimethylbutane, 2,3-dimethylbutane, cycloheptane, n-heptane, 2 methylhexane, 3-methylhexane, 2,2-dimethylpentane, 2,3-dimethylpentane, 3,3-dimethylpentane, 2,4-dimethylpentane, 3-ethylpentane, 2,3,3-trimethylbutane, cyclooctane, cyclooctene, such as cyclooctene or cis-cycloctene, n-octane, 2-methylheptane, 3-methylheptane, 4-methylheptane, 2,2-dimethylhexane, 2,3-dimethylhexane, 2,4-dimethylhexane, 2,5-dimethylhexane, 3,3-dimethylhexane, 3,4-dimethylhexane, 2,2,3-trimethylpentane, 2,2,4-trimethylpentane, 2,3,3-trimethylpentane, 2,3,4-trimethylpentane, 3-ethylhexane, 3-ethyl-3-methylpentane, 2,2,3,3-tetramethylbutane, n-nonane, 2-methyloctane, 3-methyloctane, 4-methyloctane, 2,2-dimethylheptane, 2,3-dimethylheptane, 2,4-dimethylheptane, 2,5-dimethylheptane, 2,6-dimethylheptane, 3,3-dimethylheptane, 3,4-dimethylheptane, 3,5-dimethylheptane, 4,4-dimethylheptane, 3-ethylheptane, 4-ethylheptane, 2,2,3-trimethylhexane, 2,2,4-trimethylhexane, 2,2,5-trimethylhexane, 2,3,3-trimethylhexane, 2,3,4-trimethylhexane, 2,3,5-trimethylhexane, 2,4,4-trimethylhexane, 3,3,4-trimethylhexane, 3-ethyl-2-methylhexane, 4-ethyl-2-methylhexane, 3-ethyl-3-methylhexane, 4-ethyl-3-methylhexane, 2,2,3,3-tetramethylpentane, 2,2,3,4-tetramethylpentane, 2,2,4,4-tetramethylpentane, 2,3,3,4-tetramethylpentane, 3-ethyl-2,2-dimethylpentane, 3 Ethyl 2,3-dimethylpentane, 3-ethyl-2,4-dimethylpentane, 3,3-diethylpentane, cyclodecane, n-decane, 2-methylnonane, 3-methylnonane, 4-methylnonane, 5-methylnonane, 2,2-Dimethyl octane, 2,3-dimethyloctane, 2,4-dimethyloctane, 2,5-dimethyloctane, 2,6-dimethyloctane, 2,7-dimethyloctane, 3,3-dimethyloctane, 3,4-dimethyloctane, 3,5-dimethyloctane, 3,6-dimethyloctane, 4,4-dimethyloctane, 4,5-dimethyloctane, 2,2,3-trimethylheptane, 2,2,4-trimethylheptane, 2,2,5-trimethylheptane, 2,2,6-trimethylheptane, 2,3,3-trimethylheptane, 2,3,4-trimethylheptane, 2,3,5-trimethylheptane, 2,3,6-trimethylheptane, 2,4,4-trimethylhep tan, 2,4,5-trimethylheptane, 2,4,6-trimethylheptane, 2,5,5-trimethylheptane, 3,3,4-trimethylheptane, 3,3,5-trimethylheptane, 3,4,4-trimethylheptane, 3,4,5-trimethylheptane, 2,2,3,3-tetramethylhexane, 2,2,3,4-tetramethylhexane, 2,2,3,5-tetramethylhexane, 2,2,4,4-tetramethylhexane, 2,2,4,5-tetramethylhexane, 2,2,5,5-tetramethylhexane, 2,3,3,4-tetramethylhexane, 2,3,3,5-tetramethylhexane, 2,3,4,4-tetramethylhexane, 2,3,4,5-tetramethylhexane, 3,3,4,4-tetramethylhexane, 2,2,3,3,4-pentamethylpentane, 2,2,3,4,4-pentamethylpentane, 3-ethyloctane, 4-ethyloctane, 3,3-diethylhexan, 3,4-diethylhexane, 3-ethyl-2-methylheptane, 3-ethyl-3-methylheptane, 3-ethyl-4-methylheptane, 3-ethyl-5-methylheptane, 4-ethyl-2-methylheptane, 4-ethyl 3-methylheptane, 4-ethyl-4-methylheptane, 5-ethyl-2-methylheptane, 3-ethyl-2,2-dimethylhexane, 3-ethyl-2,3-dimethylhexane, 3-ethyl-2,4-dimethylhexane, 3-ethyl-2,5-dimethylhexane, 3-ethyl-3,4-dimethylhexane, 4-ethyl-2,2-dimethylhexane, 4-ethyl-2,3-dimethylhexane, 4-ethyl-2,4-dimethylhexane, 4-ethyl-3,3-dimethylhexane, 3-ethyl-2,2,3-trimethylpentane, 3-ethyl-2,2,4-trimethylpentane, 3-ethyl-2,3,4-trimethylpentane, 3,3-diethyl2-methylpentane, 4-propylheptane, 4-(1-methylethyl)heptane, 2-methyl-3-(1-methylethyl) hexane, 2,4-dimethyl-3-(1-methylethyl) pentane, n-undecane, 2-methyldecane, 3-methyldecane, 4-methyldecane, 5-methyldecane, 2,2-dimethylnonane, 2,3-dimethylnonane, 2,4-dimethylnonane, 2,5-dimethylnonane, 2,6-dimethylnonane, 2,7-dimethylnonane, 2,8-dimethylnonane, 3,3-dimethylnonane, 3,4-dimethylnonane, 3,5-dimethylnonane, 3,6-dimethylnonane, 3,7-dimethylnonane, 4,4-dimethylnon 4,5-dimethylnonane, 4,6-dimethylnonane, 5,5-dimethylnonane, 2,2,3-trimethyloctane, 2,2,4-trimethyloctane, 2,2,5-trimethyloctane, 2,2,6-trimethyloctane, 2,2,7-trimethyloctane, 2,3,3-trimethyloctane, 2,3,4-trimethyloctane, 2,3,5-trimethyloctane, 2,3,6-trimethyloctane, 2,3,7-trimethyloctane, 2,4,4-trimethyloctane, 2,4,5-trimethyloctane, 2,4,6-trimethyloctane, 2,4,7-trimethyloctane, 2,5,5-trimethyloctane, 2,5,6-trimethyloctane, 2,6,6-trimethyloctane, 3,3,4-trimethyloctane, 3,3,5-trimethyloctane, 3,3,6-trimethyloctane, 3,4,4-trimethyloctane, 3,4,5-trimethyloctane, 3,4,6-trimethyloctane, 3,5,5-trimethyloctane, 4,4,5-trimethyloctane, 2,2,3,3-tetramethylheptane, 2,2,3,4-tetramethylheptane, 2,2,3,5-tetramethylheptane, 2,2,3,6-tetramethylheptane, 2,2,4,4-tetramethylheptane, 2,2,4,5-tetramethylheptane, 2,2,4,6-tetramethylheptane, 2,2,5,5-tetramethylheptane, 2,2,5,6-tetramethylheptane, 2,2,6,6-tetramethylheptane, 2,3,3,4-tetramethylheptane, 2,3,3,5-tetramethylheptane, 2,3,3,6-tetramethylheptane, 2,3,4,4-tetramethylheptane, 2,3,4,5-tetramethylheptane, 2,3,4,6-tetramethylheptane, 2,3,5,5-tetramethylheptane, 2,3,5,6-tetramethylheptane, 2,4,4,5-tetramethylheptane, 2,4,4,6-tetramethylheptane, 2,4,5,5-tetramethylheptane, 3,3,4,4-tetramethylheptane, 3,3,4,5-tetramethylheptane, 3,3,5,5-tetramethylheptane, 3,4,4,5-Tetramethylheptane, 2,2,3,3,4-pentamethylhexane, 2,2,3,3,5-pentamethylhexane, 2,2,3,4,4-pentamethylhexane, 2,2,3,4,5-pentamethylhexane, 2,2,3,5,5-pentamethylhexane, 2,2,4,4,5-pentamethylhexane, 2,3,3,4,4-pentamethylhexane, 2,3,3,4,5-pentamethylhexane, 2,2,3,3,4,4-hexamethylpentane, n-dodecane, 2-methylundecane, 3-methylundecane, 4-methylundecane, 5-methylundecane, 6-methylundecane, 2,2-dimethyldecane, 2,3-dimethyldecane, 2,4-dimethyldecane, 2,5-dimethyldecane, 2,6-dimethyldecane, 2,7-dimethyldecane, 2,8-dimethyldecane, 2,9-dimethyldecane, 3,3-dimethyldecane, 3,4-dimethyldecane, 3,5-dimethyldecane, 3,6-dimethyldecane, 3,7-dimethyldecane, 3,8-dimethyldecane, 4,4-dimethyldecane, 4,5-dimethyldecane, 4,6-dimethyldecane, 4,7-dimethyldecane, 5,5-dimethyldecane, 5,6-dimethyldecane, 2,2,3-trimethylnonane, 2,2,4-trimethylnonane, 2,2,5-trimethylnonane, 2,2,6-trimethylnonane, 2,2,7-trimethylnonane, 2,2,8-trimethylnonane, 2,3,3-trimethylnonane, 2,3,4-trimethylnonane, 2,3,5-trimethylnonane, 2,3,6-trimethylnonane, 2,3,7-trimethylnonane, 2,3,8-trimethylnonane, 2,4,4-trimethylnonane, 2,4,5-trimethylnonane, 2,4,6-trimethylnonane, 2,4,7-trimethylnonane, 2,4,8-trimethylnonane, 2,5,5-trimethylnonane, 2,5,6-trimethylnonane, 2,5,7-trimethylnonane, 2,5,8-trimethylnonane, 2,6,6-trimethylnonane, 2,6,7-trimethylnonane, 2,7,7-trimethylnonane, 3,3,4-trimethylnonane, 3,3,5-trimethylnonane, 3,3,6-trimethylnonane, 3,3,7-trimethylnonane, 3,4,4-trimethylnonane, 3,4,5-trimethylnonane, 3,4,6-trimethylnonane, 3,4,7-trimethylnonane, 3,5,5-trimethylnonane, 3,5,6-trimethylnonane, 3,5,7-trimethylnonane, 3,6,6-trimethylnonane, 4,4,5-trimethylnonane, 4,4,6-trimethylnonane, 4,5,5-trimethylnonane, 4,5,6-trimethylnonane, 2,2,3,3-tetramethyloctane, 2,2,3,4-tetramethyloctane, 2,2,3,5-tetramethyloctane, 2,2,3,6-tetramethyloctane, 2,2,3,7-tetramethyloctane, 2,2,4,4-tetramethyloctane, 2,2,4,5-tetramethyloctane, 2,2,4,6-tetramethyloctane, 2,2,4,7-tetramethyloctane, 2,2,5,5-tetramethyloctane, 2,2,5,6-tetramethyloctane, 2,2,5,7-tetramethyloctane, 2,2,6,6-tetramethyloctane, 2,2,6,7-tetramethyloctane, 2,2,7,7-tetramethyloctane, 2,3,3,4-tetramethyloctane, 2,3,3,5-tetramethyloctane, 2,3,3,6-tetramethyloctane, 2,3,3,7-tetramethyloctane, 2,3,4,4-tetramethyloctane, 2,3,4,5-tetramethyloctane, 2,3,4,6-tetramethyloctane, 2,3,4,7-tetramethyloctane, 2,3,5,5-tetramethyloctane, 2,3,5,6-tetramethyloctane, 2,3,5,7-tetramethyloctane, 2,3,6,6-tetramethyloctane, 2,3,6,7-tetramethyloctane, 2,4,4,5-tetra methyloctane, 2,4,4,6-tetramethyloctane, 2,4,4,7-tetramethyloctane, 2,4,5,5-tetramethyloctane, 2,4,5,6-tetramethyloctane, 2,4,5,7-tetramethyloctane, 2,4,6,6-tetra methyloctane, 2,5,5,6-tetramethyloctane, 2,5,6,6-tetramethyloctane, 3,3,4,4-tetramethyloctane, 3,3,4,5-tetramethyloctane, 3,3,4,6-tetramethyloctane, 3,3,5,5-tetra methyloctane, 3,3,5,6-tetramethyloctane, 3,3,6,6-tetramethyloctane, 3,4,4,5-tetra methyloctane, 3,4,4,6-tetra methyloctane, 3,4,5,5-tetra methyloctane, 3,4,5,6-tetramethyloctane, 4,4,5,5-tetramethyloctane, 2,2,3,3,4-pentamethylheptane, 2,2,3,3,5-pentamethylheptane, 2,2,3,3,6-pentamethylheptane, 2,2,3,4,4-pentamethylheptane, 2,2,3,4,5-pentamethylheptane, 2,2,3,4,6-pentamethylheptane, 2,2,3,5,5-pentamethylheptane, 2,2,3,5,6-pentamethylheptane, 2,2,3,6,6-pentamethylheptane, 2,2,4,4,5-pentamethylheptane, 2,2,4,4,6-pentamethylheptane, 2,2,4,5,5-pentamethylheptane, 2,2,4,5,6-pentamethylheptane, 2,2,4,6,6-pentamethylheptane, 2,2,5,5,6-pentamethylheptane, 2,3,3,4,4-pentamethylheptane, 2,3,3,4,5-pentamethylheptane, 2,3,3,4,6-pentamethylheptane, 2,3,3,5,5-pentamethylheptane, 2,3,3,5,6-pentamethylheptane, 2,3,4,4,5-pentamethylheptane, 2,3,4,4,6-pentamethylheptane, 2,3,4,5,5-pentamethylheptane, 2,3,4,5,6-pentamethylheptane, 2,4,4,5,5-pentamethylheptane, 3,3,4,4,5-pentamethylheptane, 3,3,4,5,5-pentamethylheptane, 2,2,3,3,4,4-hexamethylhexane, 2,2,3,3,4,5-hexamethylhexan, 2,2,3,3,5,5-hexamethylhexane, 2,2,3,4,4,5-hexamethylhexane, 2,2,3,4,5,5-hexamethylhexane, 2,3,3,4,4,5-hexamethylhexane, toluene, ortho-xylene(1,2-dimethylbenzene), m-xylene (1,3-dimethylbenzene), p-xylene(1,4-dimethylbenzene), 1,2,3-trimethylbenzene, 1,2,4 trimethylbenzene, 1,3,5 trimethylbenzene, THF (tetrahydrofuran), methyltetrahydrofuran, such as 2-methyltetrahydrofuran, 3-methyltetrahydrofuran, ethyltetrahydrofuran, such as 2-ethyltetrahydrofuran, 3-ethyltetrahydrofuran, MTBE (2-mehoxy-2-methylpropane) or TBME (tert-butyl methyl ether), di-n-amyl ether, tert-amyl ether, DCM (dichloromethane) or chloroform (trichloromethane), ethanol, propan-1-ol, propan-2-ol, butan-1-ol, butan-2-ol, tert-butanol, pentan-1-ol, pentan-2-ol, pentan-3-ol, 2-methyl-2-butanol, hexan-1-ol, hexan-2-ol, hexan-3-ol, 2-methyl-2-pentanol, 2-methyl-3-pentanol, 4-methyl-2-pentanol, heptan-1-ol, heptan-2-ol, heptan-3-ol, heptan-4-ol, 2-methyl 2-hexanol, octan-1-ol, octan-2-ol, octan-3-ol, octa n-4-ol, 2-methyl-2-heptanol, nonan-1-ol, nonane-2-ol, nonan-3-ol, nonan-4-ol, nonan-5-ol, 2-methyl-2-octanol, decan-1-ol, undecan-1-ol, dodecan-1-ol or DMSO (dimethyl sulfoxide). Preferably, the solvents are volatile solvents. Volatile solvents are solvents having a boiling point of at most 200° C., preferably at most 190° C., more preferably at most 180° C., more preferably at most 170° C., more preferably at most 160° C., more preferably at most 150° C., more preferably at most 140° C., more preferably at most 130° C., more preferably at most 120° C., more preferably at most 110° C., more preferably at most 100° C., more preferably at most 90° C., more preferably at most 80° C., more preferably at most 70° C., more preferably at most 60° C., and most preferably at most 50° C. at room temperature and normal pressure.

Particularly preferred solvents are n-pentane, cyclopentane, n-hexane, cyclohexane, n-heptane, n-octane, isooctane (2,2,4-trimethylpentane), ethanol, propan-1-ol, propan-2-ol, butan-1-ol, butan-2-ol, tert-butanol, pentan-1-ol, pentan-2-ol, pentan-3-ol, 2-methyl-2-butanol, hexan-1-ol, toluene, 1,2-dimethylbenzene, 1,3-dimethylbenzene, 1,4-dimethylbenzene, THF (tetrahydrofuran), 2-methyltetrahydrofuran, MTBE (2-methoxy-2-methylpropane) or TBME (tert-butylmethyl ether), tert-amyl ether, DMSO, DCM (dichloromethane) or chloroform (trichloromethane). The most preferred solvents are pentane, isooctane (2,2,4-trimethylpentane), toluene, THF (tetrahydrofuran), MTBE (2-methoxy-2-methylpropane) and TBME (tert-butyl methyl ether), ethanol, propane-1-ol, propan-2-ol, DMSO, DCM (dichloromethane) or chloroform (trichloromethane).

The solvents that can be used can also be combined with each other. The surface coating according to the invention preferably has a (generally) homogeneous layer thickness of 5 nm to 50 μm, more preferably 10 nm and 25 μm and more preferably 20 nm and 10 μm and most preferably 30 nm to 1 μm.

In a further preferred embodiment, 2- or multi-layer coatings are carried out with hydrophobic cationic electrolytes and polyelectrolytes. For this purpose, in one embodiment, following a coating, drying is carried out as described above. The coating process is then carried out with the identical hydrophobic cationic electrolyte and polyelectrolyte as before and/or with another hydrophobic cationic electrolyte and polyelectrolyte under the same or different process conditions.

In a further preferred embodiment, hydrophobic cationic electrolytes and polyelectrolytes are multi-layered, including a hydrophobic cationic electrolyte or at least one hydrophobic cationic polyelectrolyte or a mixture comprising at least one hydrophobic cationic electrolyte and at least one hydrophobic cationic polyelectrolyte and in an alternative coating step a mixture of a carboxylic acid and a hydrophobic cationic electrolyte or at least one hydrophobic cationic polyelectrolyte or a mixture of the like are consecutively or alternately applied to the material surface. Preferably, the material surfaces are subjected to drying after each coating application.

In the methods according to the invention, steps b) and c) can be repeated two or more times consecutively after step c) and before step d).

In one embodiment, the method according to the invention for producing a surface coating comprises the following steps:

a) providing a solid material,

b) wetting of the surface of the solid material under anhydrous conditions with at least one hydrophobic cationic electrolyte or at least one hydrophobic cationic polyelectrolyte or a mixture comprising at least one hydrophobic cationic electrolyte and at least one hydrophobic cationic polyelectrolyte,

c) drying the surface,

d) wetting of the surface under anhydrous conditions with at least one carboxylic acid,

e) rinsing and drying the surface,

f) obtaining the surface coating,

wherein steps b) and c) are repeated two or more times consecutively after step c) and before step d).

In a further preferred embodiment, a two-layered or multi-layered layer structure with hydrophobic cationic electrolytes and polyelectrolytes is produced by alternately coating with hydrophobic cationic electrolytes and/or polyelectrolytes and hydrophobic anionic electrolytes and/or polyelectrolytes. It is preferred if the last layer of a hydrophobic electrolyte and/or polyelectrolyte is a hydrophobic cationic electrolyte and/or polyelectrolyte.

Preference is given to a layered construction which has a overall thickness of 5 nm to 50 μm, more preferably between 10 nm and 25 μm and more preferably between 20 nm and 10 μm.

In a preferred embodiment, one or more hydrophobic electrolytes, cationic and/or polyelectrolytes are mixed with one or more carboxylic acid (s), including nitro fatty acids. This is preferably carried out in a solvent phase in which the compounds are dissolved anhydrous. After complete mixing and preferably obtaining a clear solution, the mixture containing one or more hydrophobic cationic electrolytes and/or polyelectrolyte (s) and carboxylic acids may be applied. In principle, all nonpolar solvents are suitable. The following may be used as solvents: The following alkanes including their constitutional isomers and their cyclic form pentane, hexane, heptane, octane, nonane, decane, undecane, or dodecane, benzene, toluene, xylenes, trimethylbenzene, ethers, halogenated solvents or alcohols. Specifically, it may be the following solvent treatments: cyclopentane, n-pentane, cyclohexane, n-hexane, 2-methylpentane, 3-methylpentane, 2,2-dimethylbutane, 2,3-dimethylbutane, cycloheptane, n-heptane, 2 methylhexane, 3-methylhexane, 2,2-dimethylpentane, 2,3-dimethylpentane, 3,3-dimethylpentane, 2,4-dimethylpentane, 3-ethylpentane, 2,3,3-trimethylbutane, cyclooctane, cyclooctene, such as cyclooctene or cis-cycloctene, n-octane, 2-methylheptane, 3-methylheptane, 4-methylheptane, 2,2-dimethylhexane, 2,3-dimethylhexane, 2,4-dimethylhexane, 2,5-dimethylhexane, 3,3-Dimethylhexane, 3,4-dimethylhexane, 2,2,3-trimethylpentane, 2,2,4-trimethylpentane, 2,3,3-trimethylpentane, 2,3,4-trimethylpentane, 3-ethylhexane, 3-ethyl-3-methylpentane, 2,2,3,3-tetramethylbutane, n-nonane, 2-methyloctane, 3-methyloctane, 4-methyloctane, 2,2-dimethylheptane, 2,3-dimethylheptane, 2,4-dimethylheptane, 2,5-dimethylheptane, 2,6-dimethylheptane, 3,3-dimethylheptane, 3,4-dimethylheptane, 3,5-dimethylheptane, 4,4-dimethylheptane, 3-ethylheptane, 4-ethylheptane, 2,2,3-trimethylhexane, 2,2,4-trimethylhexane, 2,2,5-trimethylhexane, 2,3,3-trimethylhexane, 2,3,4-trimethylhexane, 2,3,5-trimethylhexane, 2,4,4-trimethylhexane, 3,3,4-trimethylhexane, 3-ethyl-2-methylhexane, 4-ethyl-2-methylhexane, 3-ethyl-3-methylhexane, 4-ethyl-3-methylhexane, 2,2,3,3-tetramethylpentane, 2,2,3,4-tetramethylpentane, 2,2,4,4-tetramethylpentane. 2,3,3,4-tetramethylpentane, 3-ethyl-2,2-dimethylpentane, 3-ethyl-2,3-dimethylpentane, 3-ethyl-2,4-dimethylpentane, 3,3-diethylpentane, cyclodecane, n-decane, 2-methylnonane, 3-methylnonane, 4-methylnonane, 5-methylnonane, 2,2-dimethyloctane, 2,3-dimethyloctane, 2,4-dimethyloctane, 2,5-dimethyloctane, 2,6-dimethyloctane, 2,7-dimethyloctane, 3,3-dimethyloctane, 3,4-dimethyloctane, 3,5-dimethyloctane, 3,6-dimethyloctane, 4,4-dimethyloctane, 4,5-dimethyloctane, 2,2,3-trimethylheptane, 2,2,4-trimethylheptane, 2,2,5-trimethylheptane, 2,2,6-trimethylheptane, 2,3,3-trimethylheptane, 2,3,4-trimethylheptane, 2,3,5-trimethylheptane, 2,3,6-trimethylheptane, 2,4,4-trimethylheptane, 2,4,5-trimethylheptane, 2,4,6-trimethylheptane, 2,5,5-trimethylheptane, 3,3,4-trimethylheptane, 3,3,5-trimethylheptane, 3,4,4-trimethylheptane, 3,4,5-trimethylheptane, 2,2,3,3-tetramethylhexane, 2,2,3,4-tetramethylhexane, 2,2,3,5-tetramethylhexane, 2,2,4,4-tetramethylhexane, 2,2,4,5-tetramethylhexane, 2,2,5,5-tetramethylhexane, 2,3,3,4-tetramethylhexane, 2,3,3,5-tetramethylhexane, 2,3,4,4-tetramethylhexane, 2,3,4,5-tetramethylhexane, 3,3,4,4-tetramethylhexane, 2,2,3,3,4-pentamethylpentane, 2,2,3,4,4-pentamethylpentane, 3-ethyloctane, 4-ethyloctane, 3,3-diethylhexane, 3,4-diethylhexane, 3-ethyl-2-methylheptane, 3-ethyl-3-methylheptane, 3-ethyl-4-methylheptane, 3-ethyl-5-methylheptane, 4-ethyl-2-methylheptane, 4-ethyl-3-methylheptane, 4-ethyl-4-methylheptane, 5-ethyl-2-methylheptane, 3-ethyl-2,2-dimethylhexane, 3-ethyl-2,3-dimethylhexane, 3-ethyl-2,4-dimethylhexane, 3-ethyl-2,5-dimethylhexane, 3-ethyl-3,4-dimethylhexane, 4-ethyl-2,2-dimethylhexane, 4-ethyl-2,3-dimethylhexane, 4-ethyl-2,4-dimethylhexane, 4-ethyl-3,3-dimethylhexane, 3-ethyl-2,2,3-trimethylpentane, 3-ethyl-2,2,4-trimethylpentane, 3-ethyl-2,3,4-trimethylpentane, 3,3-diethyl-2-methylpentane, 4-propylheptane, 4-(1-methylethyl)heptane, 2-methyl-3-(1-methylethyl)hexane, 2,4-dimethyl-3-(1-methylethyl)pentane, n-undecane, 2-methyldecane, 3-methyldecane, 4-methyldecane, 5-methyldecane, 2,2-dimethylnonane, 2,3-dimethylnonane, 2,4-dimemethylnonane, 2,5-dimethylnonane, 2,6-dimethylnonane, 2,7-dimethylnonane, 2,8-dimethylnonane, 3,3-dimethylnonane, 3,4-dimethylnonane, 3,5-dimethylnonane, 3,6-dimethylnonane, 3,7-dimethylnonane, 4,4-dimethylnonane, 4,5-dimethylnonane, 4,6-dimethylnonane, 5,5-dimethylnonane, 2,2,3-trimethyloctane, 2,2,4-trimethyloctane, 2,2,5-trimethyloctane, 2,2,6-trimethyloctane, 2,2,7-trimethyloctane, 2,3,3-trimethyloctane, 2,3,4-trimethyloctane, 2,3,5-trimethyloctane, 2,3,6-trimethyloctane, 2,3,7-trimethyloctane, 2,4,4-trimethyloctane, 2,4,5-trimethyloctane, 2,4,6-trimethyloctane, 2,4,7-trimethyloctane, 2,5,5-trimethyloctane, 2,5,6-trimethyloctane, 2,6,6-trimethyloctane, 3,3,4-trimethyloctane, 3,3,5-trimethyloctane, 3,3,6-trimethyloctane, 3,4,4-trimethyloctane, 3,4,5-trimethyloctane, 3,4,6-trimethyloctane, 3,5,5-trimethyloctane, 4,4,5-trimethyloctane, 2,2,3,3-tetramethylheptane, 2,2,3,4-tetramethylheptane, 2,2,3,5-tetramethylheptane, 2,2,3,6-tetramethylheptane, 2,2,4,4-tetramethylheptane, 2,2,4,5-tetramethylheptane, 2,2,4,6-tetramethylheptane, 2,2,5,5-tetramethylheptane, 2,2,5,6-tetramethylheptane, 2,2,6,6-tetramethylheptane, 2,3,3,4-tetramethylheptane, 2,3,3,5-tetramethylheptane, 2,3,3,6-tetramethylheptane, 2,3,4,4-tetramethylheptane, 2,3,4,5-tetramethylheptane, 2,3,4,6-tetramethylheptane, 2,3,5,5-tetramethylheptane, 2,3,5,6-tetramethylheptane, 2,4,4,5-tetramethylheptane, 2,4,4,6-tetramethylheptane, 2,4,5,5-tetramethylheptane, 3,3,4,4-tetramethylheptane, 3,3,4,5-tetramethylheptane, 3,3,5,5-tetramethylheptane, 3,4,4,5-tetramethylheptane, 2,2,3,3,4-pentamethylhexane, 2,2,3,3,5-pentamethylhexane, 2,2,3,4,4-pentamethylhexane, 2,2,3,4,5-pentamethylhexane, 2,2,3,5,5-pentamethylhexane, 2,2,4,4,5-pentamethylhexane, 2,3,3,4,4-pentamethylhexane, 2,3,3,4,5-pentamethylhexane, 2,2,3,3,4,4-hexamethylpentane, n-dodecane, 2-methylundecane, 3-methylundecane, 4-methylundecane, 5-methylundecane, 6-methylundecane, 2,2-dimethyldecane, 2,3-dimethyldecane, 2,4-dimethyldecane, 2,5-dimethyldecane, 2,6-dimethyldecane, 2,7-dimethyldecane, 2,8-dimethyldecane, 2,9-dimethyldecane, 3,3-dimethyldecane, 3,4-dimethyldecane, 3,5-dimethyldecane, 3,6-dimethyldecane, 3,7-dimethyldecane, 3,8-dimethyldecane, 4,4-dimethyldecane, 4,5-dimethyldecane, 4,6-dimethyldecane, 4,7-dimethyldecane, 5,5-dimethyldecane, 5,6-dimethyldecane, 2,2,3-trimethylnonane, 2,2,4-trimethylnonane, 2,2,5-trimethylnonane, 2,2,6-trimethylnonane, 2,2,7-trimethylnonane, 2,2,8-trimethylnonane, 2,3,3-trimethylnonane, 2,3,4-trimethylnonane, 2,3,5-trimethylnonane, 2,3,6-trimethylnonane, 2,3,7-trimethylnonane, 2,3,8-trimethylnonane, 2,4,4-trimethylnonane, 2,4,5-trimethylnonane, 2,4,6-trimethylnonane, 2,4,7-trimethylnonane, 2,4,8-trimethylnonane, 2,5,5-trimethylnonane, 2,5,6-trimethylnonane, 2,5,7-trimethylnonane, 2,5,8-trimethylnonane, 2,6,6-trimethylnonane, 2,6,7-trimethylnonane, 2,7,7-trimethylnonane, 3,3,4-trimethylnonane, 3,3,5-trimethylnonane, 3,3,6-trimethylnonane, 3,3,7-trimethylnonane, 3,4,4-trimethylnonane, 3,4,5-trimethylnonane, 3,4,6-trimethylnonane, 3,4,7-trimethylnonane, 3,5,5-trimethylnonane, 3,5,6-trimethylnonane, 3,5,7-trimethylnonane, 3,6,6-trimethylnonane, 4,4,5-trimethylnonane, 4,4,6-trimethylnonane, 4,5,5-trimethylnonane, 4,5,6-trimethylnonane, 2,2,3,3-tetramethyloctane, 2,2,3,4-tetramethyloctane, 2,2,3,5-tetramethyloctane, 2,2,3,6-tetramethyloctane, 2,2,3,7-tetramethyloctane, 2,2,4,4-tetramethyloctane, 2,2,4,5-tetramethyloctane, 2,2,4,6-tetramethyloctane, 2,2,4,7-tetramethyloctane, 2,2,5,5-tetramethyloctane, 2,2,5,6-tetramethyloctane, 2,2,5,7-tetramethyloctane, 2,2,6,6-tetramethyloctane, 2,2,6,7-tetramethyloctane, 2,2,7,7-tetramethyloctane, 2,3,3,4-tetramethyloctane, 2,3,3,5-tetramethyloctane, 2,3,3,6-tetramethyloctane, 2,3,3,7-tetramethyloctane, 2,3,4,4-tetramethyloctane, 2,3,4,5-tetramethyloctane, 2,3,4,6-tetramethyloctane, 2,3,4,7-tetramethyloctane, 2,3,5,5-tetramethyloctane, 2,3,5,6-tetramethyloctane, 2,3,5,7-tetramethyloctane, 2,3,6,6-tetra methyloctane, 2,3,6,7-tetramethyloctane, 2,4,4,5-tetramethyloctane, 2,4,4,6-tetra methyloctane, 2,4,4,7-tetra methyloctane, 2,4,5,5-tetramethyloctane, 2,4,5,6-tetramethyloctane, 2,4,5,7-tetramethyloctane, 2,4,6,6-tetramethyloctane, 2,5,5,6-tetra methyloctane, 2,5,6,6-tetra methyloctane, 3,3,4,4-tetra methyloctane, 3,3,4,5-tetramethyloctane, 3,3,4,6-tetramethyloctane, 3,3,5,5-tetramethyloctane, 3,3,5,6-tetra methyloctane, 3,3,6,6-tetra methyloctane, 3,4,4,5-tetra methyloctane, 3,4,4,6-tetramethyloctane, 3,4,5,5-tetramethyloctane, 3,4,5,6-tetramethyloctane, 4,4,5,5-tetramethyloctane, 2,2,3,3,4-pentamethylheptane, 2,2,3,3,5-pentamethylheptane, 2,2,3,3,6-pentamethylheptane, 2,2,3,4,4-pentamethylheptane, 2,2,3,4,5-pentamethylheptane, 2,2,3,4,6-pentamethylheptane, 2,2,3,5,5-pentamethylheptane, 2,2,3,5,6-pentamethylheptane, 2,2,3,6,6-pentamethylheptane, 2,2,4,4,5-pentamethylheptane, 2,2,4,4,6-pentamethylheptane, 2,2,4,5,5-pentamethylheptane, 2,2,4,5,6-pentamethylheptane, 2,2,4,6,6-pentamethylheptane, 2,2,5,5,6-pentamethylheptane, 2,3,3,4,4-pentamethylheptane, 2,3,3,4,5-pentamethylheptane, 2,3,3,4,6-pentamethylheptane, 2,3,3,5,5-pentamethylheptane, 2,3,3,5,6-pentamethylheptane, 2,3,4,4,5-pentamethylheptane, 2,3,4,4,6-pentamethylheptane, 2,3,4,5,5-pentamethylheptane, 2,3,4,5,6-pentamethylheptane, 2,4,4,5,5-pentamethylheptane, 3,3,4,4,5-pentamethylheptane, 3,3,4,5,5-pentamethylheptane, 2,2,3,3,4,4-hexamethylhexane, 2,2,3,3,4,5-hexamethylhexane, 2,2,3,3,5,5-hexamethylhexane, 2,2,3,4,4,5-hexamethylhexane, 2,2,3,4,5,5-hexamethylhexane, 2,3,3,4,4,5-hexamethylhexane, toluene, ortho-xylene(1,2-dimethylbenzene), m-xylene (1,3-dimethylbenzene), p-xylene (1,4-dimethylbenzene), 1,2,3-trimethylbenzene, 1,2,4-trimethylbenzene, 1,3,5-trimethylbenzene, THF (tetrahydrofuran), methyltetrahydrofuran such as 2-methyltetrahydrofuran, 3-methyltetrahydrofuran, ethyltetrahydrofuran such as 2-ethyltetrahydrofuran, 3-ethyltetrahydrofuran, MTBE (2-methoxy-2-methylpropane) or TBME (tert-butylmethylether), di-n-amyl ether, tert-amyl ether, DCM (dichloromethane) or chloroform (trichloromethane), ethanol, propan-1-ol, propan-2-ol, butan-1-ol, butan-2-ol, tert-butanol, pentan-1-ol, pentan-2-ol, pentan-3-ol, 2-methyl-2-butanol, hexan-1-ol, hexan-2-ol, hexan-3-ol, 2-methyl-2-pentanol, 2-methyl-3-pentanol, 4-methyl-2-pentanol, heptan-1-ol, heptan-2-ol, heptan-3-ol, heptan-4-ol, 2-methyl-2-hexanol, octan-1-ol, octan-2-ol, octan-3-ol, octan-4-ol, 2-methyl-2-heptanol, nonan-1-ol, nonan-2-ol, nonan-3-ol, nonan-4-ol, nonan-5-ol, 2-methyl-2-octanol, decan-1-ol, undecan-1-ol or dodecan-1-ol. Preferably, the solvents are volatile solvents. Volatile solvents are solvents having a boiling point of preferably at most 140° C., more preferably at most 130° C., further preferably at most 120° C., more preferably at most 110° C., more preferably at most 100° C., more preferably at most 90° C. more preferably at most 80° C., more preferably at most 70° C., more preferably at most 60° C., and most preferably at most 50° C. at room temperature and normal pressure.

Particularly preferred solvents are n-pentane, cyclopentane, n-hexane, cyclohexane, n-heptane, n-octane, isooctane (2,2,4-trimethylpentane), ethanol, propan-1-ol, propan-2-ol, butan-1-ol, butan-2-ol, tert-butanol, pentan-1-ol, pentan-2-ol, pentan-3-ol, 2-methyl-2-butanol, hexane-1-ol, toluene, 1,2-dimethylbenzene, 1,3-dimethylbenzene, 1,4-dimethylbenzene, THF (tetrahydrofuran), 2-methyltetrahydrofuran, MTBE (2-methoxy-2-methylpropane) and TBME (tert-butyl methyl ether), tert-amyl ether, DCM (dichloromethane) or chloroform (trichloromethane). The most preferred solvents are pentane, isooctane (2,2,4-trimethylpentane), toluene, THF (tetrahydrofuran), MTBE (2-methoxy-2-methylpropane) or TBME (tert-butyl methyl ether), DCM (dichloromethane) or chloroform (trichloromethane).

The application of one or a mixture of a several hydrophobic cationic electrolytes and/or polyelectrolytes or a mixture with one or more carboxylic acids may be carried out by methods known in the art. Preference is given to a dip-coating in which the substrate to be coated is placed in a bath of the hydrophobic cationic electrolytes and/or polyelectrolytes and optionally carboxylic acids or immersed in this using a holding device. Preference is given to exposure over 10 seconds to 24 hours, more preferably between 20 seconds and 10 hours and more preferably between 30 seconds and 5 hours. Further preferred wetting methods are, for example, a spray coating or a spin coating.

Preferred is a drying following a coating. Preferably, this is done by means of a vacuum drying process.

The application procedure can be repeated or combined with another. The completeness of the coating can be tested by prior art techniques, such as ellipsometry or confocal laser microscopy.

### Method for Carrying Out Step c)

The coated material surfaces done in step b) are freed of herein/hereby/adhering residues of solvents in step c). For this purpose, methods of the prior art can be used. Preference is given to vacuum drying, which preferably takes place over a period of 10 minutes to 24 hours, more preferably between 20 minutes and 10 hours. The drying was successful when no residues of the used solvents can be detected by analytical methods; otherwise the drying period must be extended. The resulting coated material is stored in an anhydrous atmosphere until the next treatment step. Storage in a vacuum or an inert gas in preferred.

### Method for Carrying Out Step d)

The application of one or more different carboxylic acid (s) is preferably carried out under protective gas conditions using prior art methods. For this purpose, the one or more carboxylic acid (s) are completely dissolved in a suitable solvent. Preference is given, for example, to low-polar solvents, such as methanol or acetone. The materials to be coated are then preferably coated with a dipping process. This is preferably carried out over a period of 1 minute and 24 hours, more preferably between 2 minutes and 1 hour and even more preferably between 3 and 10 minutes. The temperature at which this takes place can be chosen freely, preferably a temperature between 5° and 50° C., more preferably between 10° and 30° C.

In one embodiment, electrical contact is made with the preferably metallic coated material and a voltage is applied between the material to be coated and the coating solution or coating device. Alternatively, an electrostatic charge is applied to the surface. The positively charged cationic compounds of the coated hydrophobic cationic polyelectrolytes under these conditions are preferably wetted with the dissolved carboxylic acids in a bath.

The electrostatically bound carboxylic acids are preferably present as a monolayer with a longitudinal axis of the carboxylic acid aligned perpendicular to the substrate plane. The applied layer preferably has a thickness which corresponds to a molecular length of the coating-related carboxylic acid. In further preferred embodiments, the carboxylic acids are applied in more than one layer. For this purpose, the 2nd, 4th, 6th, etc. coating can be carried out with carboxylic acids from an aqueous medium. The 3rd, 5th, 7th, etc. coating layer is then performed using an anhydrous solvent phase. The application of the second and further layers can be carried out by the methods described herein. The successful surface coating can be done with prior art methods. The determination of the water contact angle is preferred. A successful coating is when the water contact angle is >80°, more preferably 2 90°, more preferably >100°, more preferably ≥110° and even more preferably ≥120°. The data refer to a measurement at 20° C. and normal pressure conditions.

The anhydrous wetting of the surface with at least one carboxylic acid is carried out using an organic solvent such as ethanol, methanol or acetone.

### Method for Carrying Out Step e)

In this process step, the material surfaces coated with carboxylic acid in step d) are freed of the adsorptively adhering carboxylic acids which are not electrostatically associated with the cationic electrolytes or polyelectrolytes. For this purpose, the coated materials are preferably inserted or rinsed in a bath. Preferably, this is done with an alcoholic solution, preferably a mixture of water and methanol, further preferred are nonpolar organic solvents, such as ethyl acetate or acetone. The rinsing or washing step can be repeated. Subsequently, careful removal of residual solvent is performed, which is preferably carried out by vacuum drying.

### Method of Quality Testing of Step f)

The surfaces produced according to the invention are hydrophobic and have a water contact angle of ≥80° or >80°, more preferably ≥90° or >90°, more preferably ≥100° or >100, more preferably ≥110° or >110° and most preferably ≥120° or >120°. There is also an electrical insulation having a surface resistance of >200 ohms/cm², preferably ≥300 ohms/cm², more preferably ≥400 ohms/cm², more preferably ≥500 ohms/cm², even more preferably ≥1,000 ohms/cm², more preferably ≥1,100 ohms/cm², even more preferably >1,200 ohms/cm², still more preferably >1,300 ohms/cm², still more preferably ≥1,400 ohms/cm², still more preferably ≥1,500 ohms/cm², more preferably ≥1,600 ohms/cm², even more preferably >1,800 ohms/cm², even more preferably ≥1,900 ohms/cm², still more preferably ≥2,000 ohms/cm², still more preferably ≥2,500 ohms Furthermore, the surfaces stable against degradation in an aqueous medium at 37° C. for 4 weeks, and there is a change in the water contact angle of <10° during this period. Furthermore, contact with living cells with the surfaces coated according to the invention results in adhesion, and the adhering cells have a significantly lower proliferation rate with respect to the cell adhering to a native (uncoated) material surface, with a low rate of apoptosis or necrosis.

### Applications

In principle, a surface coating according to the invention can be applied to all solid medical instruments, implants and wound materials. A surface coating according to the invention is particularly suitable for those implants whose structuring materials are to decompose, dissolve and/or degrade or dissolve in the course.

This applies in particular to all resorbable materials, in particular vascular implants, such as scaffolds, furthermore surgical meshes, expanders, suture and staple materials. With one of the methods disclosed herein, the time to onset of degradation/corrosion may be significantly delayed. As a result, degradable materials retain their shape and functional properties for longer. Furthermore, coatings according to the invention are suitable for suppressing corrosion processes and thereby eliminating a detachment of degradation products. Therefore, applications in metallic implants such as stents or osteosynthesis materials are advantageous. Furthermore, a surface coating according to the invention is particularly suitable for controlling active ingredients and/or supportive compounds and releasing them to the surrounding tissue over a relatively long period of time. This may be particularly advantageous, for example, in very extensive wound areas or wounds with a high inflammatory activity, such as burns. Thus, in particular, a coating of wound care materials is advantageous. The coating process is particularly suitable for binding hydrophobic compounds and releasing them in a controlled manner. Therefore, a coating method according to the invention is also particularly suitable for the local release of hydrophobic active substances for reducing or suppressing a re-narrowing of the vessels, in particular taxols and rapamycin as well as their derivatives. Furthermore, a method according to the invention is suitable for forming tissue structures on artificial moldings or for producing endothelized vessel structures. Furthermore, a coating according to the invention on implants is particularly advantageous when a rapid adhesion of cells is desired, such as in intraocular lenses. Furthermore, a coating according to the invention is suitable for inhibiting the attachment of coagulation factors and platelets, as for example in catheters of venous ports or blood tubing systems as well as vascular implants.

### FIGURE DESCRIPTION

FIG. 1: shows Table 1 containing the numerical results from Example 1.

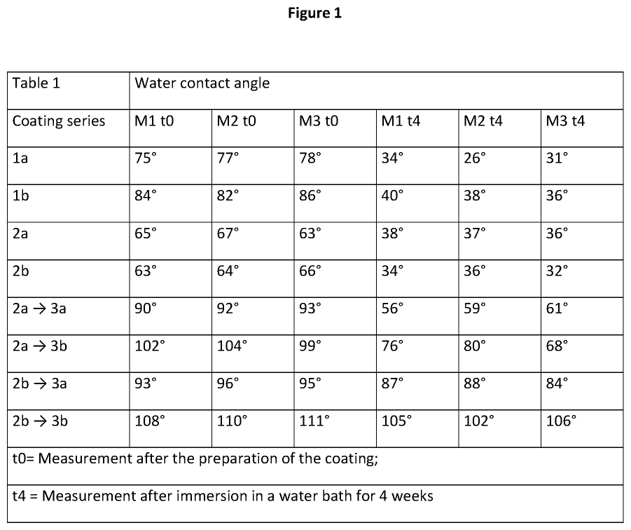

FIG. 2: shows Table 2 containing the numerical results from Example 3.

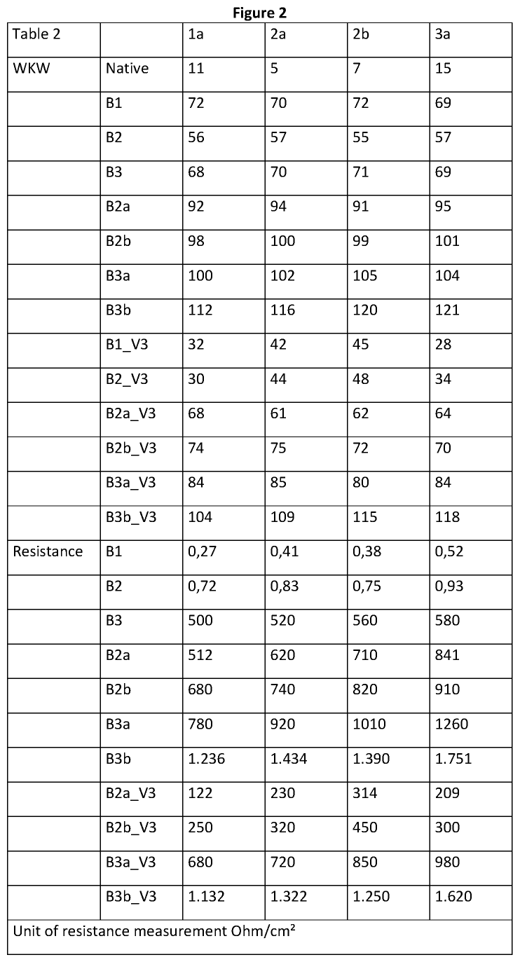

FIG. 3: shows Table 3 containing the numerical results from Example 9.

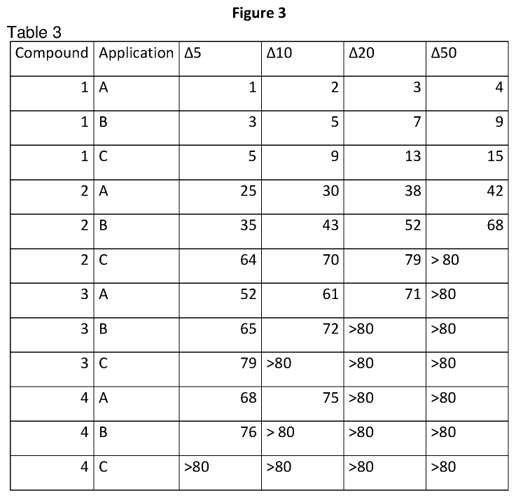

### EXAMPLES

### Measurement Methods

The following measuring methods were used in the context of the exemplary embodiments described below:

The content of phosphorus, calcium, magnesium and iron in the water phases was determined by ICP OES (iCAP 7400, Thermo-Fisher, Scientific, Germany). Values in ppm (or in mg/kg).

All coatings were done at room temperature (20° C.) and normal pressure conditions unless otherwise stated.

Water contact angle measurements were made with an automated contact angle meter (DAS 30, Kruss, Germany). The measurements were made by the sessile drop method, after 1 second contact time, at 20° C.

### Example 1

### Investigation of the Coatability of Materials.

For the examination, preparations of a surgical mesh made of PLLA (M1), a vascular scaffold (Resoloy, MeKo, Germany) (M2) and a template of osteosynthesis material (316L stainless steel) (M3) were used. The pieces of material were joined in at least 2 places with a platinum wire, which was attached by soldering or thermomodulation. These had the task of securing the pieces of material during the coating procedure, so that there was no contact of the material surfaces to a support surface in a container. Furthermore, they were used to measure the insulation resistance of the coated material surfaces (M2 and M3). The materials were cleaned in DCM in an ultrasonic bath for 20 minutes. This was followed by drying in a nitrogen stream and subsequent storage in an inert atmosphere. The M2 preparations were placed in methanol in which L-dopamine was dissolved at a concentration of 10% by weight over 6 hours, then rinse with THF and ‘dried’ in vacuo. The M3 preparations were pretreated with oxygen plasma and then stored in toluene containing ODTMS (n-octadecyltrimethoxysilane C₂₁H₅₂O₃Si) at a concentration of 5% by weight for 6 hours. Thereafter the material was rinsed with ethanol and dried at 120° C. for 3 hours. The prepared substrates were further treated according to the following series of coatings with: 1a) oleic acid, 1b) nitro-oleic acid with 25% by volume ethanol added, 2a) polyethyleneimine (MW 20,000 Da) with 25% by volume methanol added, 2b) polyalkylene polyamine (MW 50,000 Da) with 20% by volume toluene added.

The surface wetting was carried out by immersion in the solutions at 25° C. for 3 hours. Then the surfaces were rinsed with ethanol. Thereafter, they were dried in a vacuum drying oven for 10 hours.

Substrates obtained from the coating steps 2a) and 2b) were coated in coating series 3a with oleic acid and in 3b series with nitro-oleic acid by immersing them in the solutions as in experimental series 1, followed by rinsing and drying.

In order to determine the electrical insulation resistance, the substrates of the individual test series were immersed in a 0.9% NaCl solution to the extent that the connection points to the platinum electrodes remained above the water surface; the determination was carried out applying a 12V DC voltage system. Subsequently, the substrates were completely immersed in the solution and left therein for 4 weeks. The determination of the electrical insulation resistance was repeated at intervals of 1 week under the identical conditions. Finally the substrate surfaces were dried. The substrate surfaces were examined by SEM, EDX and confocal laser microscopy. For carrying out confocal laser microscopy, the surfaces were immersed in a solution containing Sudan red for one hour and then rinsed with an ethanolic solution. Furthermore, the water contact angles were determined before and after a coating and after the long-term investigation.

### Results:

For the materials, the following starting water contact angles were present: M1: 82°, M2: 21°, M3: 12°. After hydrophobing, the water contact angles were 95° for M2 and 81° for M3. The other water contact angle results are shown in Table 1. Insulation resistance values were between 0.1 and 0.8 ohms for material preparations M3. The insulation resistance, which was determined after hydrophobization, was between 0.7 and 1.2 ohms. After coatings according to 1a and 1b the insulation resistance increased by 32+/−14 ohms and 55+/−12 ohms, respectively. After coatings according to 2a and 2b the insulation resistance was increased by 26+/−16 ohms and 62+/−14 ohms, respectively, compared to the untreated surface. For the material samples used in treatment series 3a or 3b the insulation resistances were between 400 to 480 and 500 to 620 ohms, whereby the resistances were 80 to 120 ohms higher when using nitro-fatty acids. After 4 weeks of storage in the water bath (t4), the insulation resistance for coating 3a was lower than at the time of manufacture (t0) by 36 to 68 ohms, while in coatings according to 3b there was a reduction of the insulation resistance, which was less than 10% of that present at time t0. A layer thickness of 18 to 35 nm was determined for the coatings according to 3a and 3b. In electron microscopy, all surfaces were free from superficial substance defects.

At time t4, there was an identical result for the coatings 3a. and 3b. after coating according to 2b). On the other hand, significant substance defects in the material surfaces were represent after coating according to 1 and 2 and to a lesser extent after coating 2a). Using confocal laser microscopy, a homogeneous and defect-free distribution of the lipophilic chromophore were documented for the coatings according to 3a and 3b after coating 2b). This was also the case in 3b at time t4, while in 3a the signal intensity was reduced.

### Example 2

### Synthesis of Hydrophobic Cationic Electrolytes and Polyelectrolytes

To prepare hydrophobic cationic electrolytes, the following compounds were used: polyethyleneimine or poly (2-aminoethyl methacrylate), each was reacted with 1-iodo-dodecane.

Compound 1: Reaction of PEI with 1-Iodododecane to PEI-C12 (100%)

### Alkylation/Hydrophobization (Hydrophobization) of Polyethyleneimine

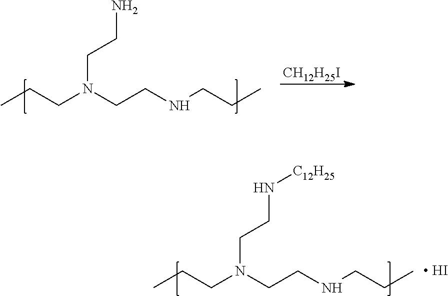

A 2.10 g (0.21 mmol) aliquot of PEI (branched, M ˜10,000 g/mol) is placed in a 50 mL rolled rim glass vial and dissolved in 10 mL ethanol with the aid of an ultrasonic bath and occasional stirring. Then 4.06 mL/4.88 g (16.5 mmol) of 1-iodododecane is added with stirring to the clear, slightly viscous solution. After 5 minutes, an additional 1.5 g (17.9 mmol) of NaHCO₃ is added and the suspension is heated to 70° C. for 15 hours. Afterwards, the suspension is as completely as possible transferred in a 50 mL centrifuge tube with 35 mL distilled water. This is a white viscous mass. After centrifugation, the supernatant is decanted off and discarded. The now turbid yellow mass is then triturated in the centrifuge tube and 35 mL fresh distilled water is added. Excess salts (sodium iodide (NaI) and sodium bicarbonate) are now extracted in an ultrasonic bath. After renewed centrifugation, the supernatant is discarded. This cleaning step is repeated for a total of four times. The resulting waxy pale yellow mass is coarsely crumbled and predried on filter paper in air and then transferred to a 50 mL centrifuge tube.

For complete removal of the residual water, the mass is first dried for 20 hours at room temperature and then for a further 20 h at 80° C. under vacuum. The resulting 3.87 g clear yellowish and sticky mass is thereby obtained (PEI-C12 (100%), yielding 79% based on the molecular weight of the product of 23,263 g/mol). The product is a honey-like or resin-like but translucent mass.

Commercially available PEI has an average molar mass of 10,000 g/mol. The molar mass of the monomer unit is 43.07 g/mol (empirical formula C₂H₅N₂). This gives an estimate of the number of monomer units per PEI molecule (232.18 monomer units per PEI molecule).

$$\frac{10000 g \text{/} mol}{43.07 g \text{/} mol} = 232.18 monomer units per PEI \text{-} molecule$$

According to the manufacturer/distributor, the PEI has a composition of 33.8% primary (RNH₂), 40.5% secondary (R₂NH) and 25.7% tertiary amino (R₃N) groups. The degree of alkylation is based on the number of primary amino groups present. A sample calculation:

$$2.1 g PEI = \frac{2.1 g}{10000 \frac{g}{mol}} = 0.21 mmol$$

This corresponds to about 16.5 mmol of primary amino groups:

0.21 mmol+232.18*0.338=16.5 mmol

This yields the amount of alkylating reagent (Alk) 1-iodododecane needed for 2.1 g PEI:

$${M}_{Alk} = 296.23 \frac{g}{mol}$$

$${ρ}_{Alk} = 1.201 g \text{/} mL$$

The amount of alkylating reagent used and the defined degree of alkylation x (1=100%) is:

$$\frac{16.5 mmol * 296.23 g \text{/} mol}{1.201 g \text{/} L} * x = 4.06 mL$$

Compound 2: Reaction of PEI with 1-Iodododecane to PEI-C12 (90%)

The reaction is carried out analogous to the preparation of compound 1, using 3.65 ml (14.9 mmol) of 1-iododecane. The product PEI-C12 (90%) is obtained a yield of 73%.

Compound 3: Reaction of PEI with 1-Iodododecane to PEI-C12 (80%)

The reaction is carried out analogous to the preparation of compound 1, using 3.25 ml (13.2 mmol) of 1-iododecane. The product PEI-C12 (80%) is obtained in a yield of 73%.

Compound 4: Reaction of PEI with 1-Iodododecane to PEI-C12 (60%)

The reaction is carried out analogous to the preparation of compound 1, using 2.44 ml (9.9 mmol) of 1-iododecane. The product PEI-C12 (60%) is obtained in a yield of 78%.

Compound 5: Reaction of PEI with 1-Iodododecane to PEI-C12 (50%)

The reaction is carried out analogous to the preparation of compound 1, using 2.03 ml (8.25 mmol) of 1-iododecane. The product PEI-C12 (50%) is obtained in a yield of 67%.

Compound 6: Reaction of PEI with 1-Iodododecane to PEI-C8 (90%)

The reaction is carried out analogous to the preparation of compound 1, using 2.68 ml of 1-iodooctane (14.85 mmol, density 1.33 g/ml, M=240.13). The product PEI-C8 (80%) is obtained in a yield of 76%.

Compound 7: Reaction of PEI with 1-Iodododecane to PEI-C8 (80%)

The reaction is carried out analogous to the preparation of compound 1, using 2.38 ml of 1-iodooctane (13.2 mmol, density 1.33 g/ml, M=240.13). The product PEI-C8 (80%) is obtained in a yield of 76%.

Compound 8: Reaction of Poly (2-Aminoethyl Methacrylate) with 1-Iodododecane (Corresponds to 100% Alkylation)

A 2.00 g (0.18 mmol) aliquot of poly (2-aminoethyl methacrylate) (M ˜11,000 g/mol) is placed in a 50 mL rolled rim glass vial and dissolved in 10 mL ethanol with the aid of an ultrasonic bath and occasional stirring, then 3.77 mL/4.53 g (15.3 mmol) of 1-iodododecane is added with stirring to the clear, slightly viscous solution. After 5 minutes, an additional 1.35 g (16.1 mmol) of NaHCO₃ is added and the suspension is heated to 70° C. for 15 hours. Afterwards, the suspension is transferred as completely as possible to a 50 mL centrifuge tube with 35 mL distilled water. This results in a viscous mass. After centrifugation, the supernatant is decanted and discarded. The now turbid mass is then transferred to the centrifuge tube and then 35 mL fresh distilled water is added. Excess salts (NaI and NaHCO₃) are now extracted in an ultrasonic bath. After renewed centrifugation, the supernatant is discarded. This cleaning step is repeated for a total of four times.

The resulting waxy mass is coarsley crumbled and predried in air on a filter paper and then transferred to a 50 mL centrifuge tube. For complete removal of the residual water, the mass is first dried for 20 hours at RT and then for a further 20 h at 80° C. in vacuo. This results in 3.43 g of a clear and sticky mass (compound 2, 75% yield based on the molecular weight of the product of 25,392 g/mol).

### Example 3

### Investigation of the Corrosion Behavior and Biological Effects of Coated Metal Materials.

The following materials in the form of wafers (substrates) of 1 cm² were examined after electropolishing and surface cleaning with acetone and toluene in an ultrasonic bath: 1a not stainless steel, also stainless steels: 2a: 316 L, 2b: Ti6AL4V, furthermore light metals: 3a: Al₂O₃.

In the coating step B1, hydrophobic treatment was carried out by immersing the preparations in a solution of toluene with ODTMS (5% by weight) and submerged therein for 6 hours followed by drying at 110° C. in a drying oven for 4 hours. In coating step B2, the preparations stored as in Example 1 were immersed for 3 hours in ethanol containing 50% by weight dissolved PEI (MW 25,000 Da, highly branched). In coating step B3, wetting with compound 1 according to Example 2 (PEI-C12 (100%)) was carried out as a 5% solution in pentane. Then the preparations were rinsed with ethanol and dried in a vacuum oven at 60° C. for 12 hours. In coating steps B2a or B3a and B2b or B3b, preparations were coated with oleic acid and nitro-oleic acid, as described in Example 1.

The coated substrates were immersed in an aggitated bath of an aqueous solution containing one of the following additives in a series of experiments performed at each coating step: V1) collagen type 1 (50 μg/ml) in sodium bicarbonate buffer for 20 minutes, V2) platelet-rich blood plasma for 24 hours, and V3) PBS with a pH of 7.4 for 4 weeks. Contact angle measurements were made after each coating step, and the surfaces were examined by SEM and confocal laser microscopy (as in Example 1) after completion of test series V1 to V3. Surfaces that had been contacted with cells and/or biomolecules were examined by fluorescence microscopy after incubation with specific fluorescent antibodies and the respective relative coverage rate and the coverage intensity (rate) were determined. Further, a light microscopic examination was performed to analyze the platelet coverage according to the following criteria: coverage density (low/medium/high), aggregate formation (none/slight/distinct), and morphology (globular/branched/spread).

The electrical resistance was determined after each coating step and after completion of V3, as described in Example 1.

The storage media after completion of V3 were analyzed for the presence of the elements aluminum, iron, chromium, cobalt and nickel as well as vanadium.

### Results:

The numerical results for the water contact angle and the insulation resistances are listed in Table 2.

On purified starting materials collagen occupied between 72 and 85% of the surface. After B1, this was between 84% and 92%, after B2 65% to 75%, after B3 34% to 45% and after B2a 15% to 25%, after B2b 8% to 21%, after B3a 5% to 12% and after B3b 5% to 10%. Further results are provided in Table 3.

In investigations on platelet adhesion (Table 4), the purified starting materials showed a medium to strong coverage, as well as slight to clear aggregates of predominantly branched platelets. After B1 there was slight occupancy, a predominantly slight aggregate formation and spherical to branched platelets. After B2, this study showed results comparable to the purified starting materials. After B3 there was a slight coverage with occasional aggregates, with predominantly fewer branched platelets. After coating, the preparations with oleic acid showed a lower coverage with platelets, which, however, still showed signs of activation. This was only a minimal proportion after coating with a cationic polyelectrolyte and was no longer present when the sample had been previously coated with a hydrophobic cationic polyelectrolyte.

In the electrolyte solutions of test series V3, metal ions or metal oxides were detectable in preparations which had been coated with coating steps B1 and B2: iron in 1a and 1b (+++) and in 2a and 2b (+), aluminum in 3a and 3b (++), cobalt in 2a (+), vanadium in 2b (+), nickel in 2a (+). In the scanning electronic investigations of the substrates of the test series V, numerous superficial defects were present after coating steps B1 and B2 3. At B3, no defects were detectable.

TABLE 4
Coverage rate for collagen and platelets

| | | Thrombocytes | |
|---|---|---|---|
| | Collagen | Aggregate | |
| | Coverage | Degree of | |
| Procedural | rate | aggregate | |
| step | [%] | formation | Form |
| Cleaned | 72-85 | +++ to ++++ | highly |
| substrate | | | branched |
| B1 | 84 - 92 | ++ | branched |
| | | | spherical form |
| B2 | 65-75 | +++ to ++++ | highly |
| | | | branched |
| B3 | 34 - 45 | + | rarely |
| | | | branched |
| B2a (oleic | 15-25 | ++ | rarely |
| acid) | | | branched |
| B2b (nitro | 8 - 21 | + | spherical form |
| oleic acid) | | | |
| B3a | 5 - 12 | + | rarely |
| | | | branched |
| B3b (nitro | 5 - 10 | + | only spherical |
| oleic acid) | | | form |

Platelet coverage: + little to no; ++ light; +++ middle coverage; ++++ high coverage.

### Example 4

### Investigation of Biological Effects of Surface Coatings.

Magnesium alloy (M1) and Teflon (M2) wavers were treated by the following coating method: 1. Surface cleaning with DCM and cyclohexane; 2. immersion in a 1:1 (wt %) mixture of THF and hydrophobized PEI (100,000 Da rarely branched, in which hydrophobization was prepared according to instructions for compound 7 according to Example 2) for 6 hours, then rinsing with methanol and repeating the procedure twice after drying for 24 hours at 60° C., 3. spray coating with a. oleic acid or b. nitro-oleic acid diluted to 10% by volume with diethyl ether. Then rinse the surfaces with ethanol and dry in a vacuum oven at 40° C. for 24 hours. For quality control, a water contact angle is measured. Native discs as well as discs after obtained 3a. and 3b. were placed at a 45° angle to the bottom in a culture vessel. The L929 fibroblasts in a nutrient medium were added to the culture vessels and the culture vessel continuously moved.

Over a period of 4 weeks, discs are taken out every 7 days for light microscopic quantification. For this purpose, the discs were first rinsed with a physiological solution and fixed in a formalin bath and stained after drying. The degree of surface coverage, cell morphology and the presence of multiple cell layer formations were assessed.

### Results:

The water contact angles obtained with the coating were between 85° and 91° for 3a and 103° to 110° for b2. On native surfaces of M1 there was a rapid adhesion of cells; the coverage rate of the surfaces was max. 30% in the first 2 weeks. This decreased over the course of time with progressive dissolution of the material. Adherent cells were highly branched. Practically no cells adhered to native surfaces of M2. Surfaces prepared in accordance with 3a. (M1 and M2) showed an increasing number of adherent cells with strong branching over the first 2 weeks. After 3 weeks, cell coverage of the surfaces was complete and multiple layers had formed. This continued until the completion of the investigation with the cells still having predominantly spindle shapes. On surfaces which had been coated according to 3b there was a significantly higher coverage rate of adherent cells in the first week, as was the case in the coating according to 3a, in which about 60% of the total area was covered. After 2 weeks there was a flat continuous cell mono-layer without multilayer formation; cells were partly flat shaped and partly rounded. Also in the further course there was only occasional formation of multiple layers; the cells had predominantly flat with broad-based adherence.

### Example 5

### Investigation of the Degradation Control of Coatings.

The controllability of the degradation resistance of the coating according to the invention was investigated. For this purpose, the effect of reservoir formation for carboxylic acids introduced into the coating was investigated by introduction of a 1-layer coating in test series D1 with A) PEI 25,000 Da in which a degree of alkylation of 50% with a C12 alkyl residues was present (rendered hydrophobic with a C12 alkyl residues) or with B) PEI 25,000 Da, which had a degree of alkylation of 90% according to Example 2. In test series D1c, in each case substance A) and B) was added with a) oleic acid in a molar ratio of the carboxyl group to non-alkylated nitrogen atoms of 1.5:1 and in series D1d) nitro-oleic acid was added in a similar manner. The solution was in a mixture of pentane.

In test series D2 and D3, a two-layer coating was investigated, in which in series D2) first a single-layer of the hydrophobic cationic polyelectrolyte was applied and, after drying, a coating with the respective carboxylic acid was carried out analogous to test series D1c and Did, and in the test series D3 the coating in D2 was carried out in reverse order. In order to produce a reservoir, after drying all the coating series were finally coated with the respective carboxylic acid which was applied in each case by spraying it with a 5% solution in methanol. Then the samples were dried. Discs of brass (substrate 1) and iron (substrate 2) were coated using the coating process. The coated substrates were placed in a physiological NaCl solution for 8 weeks. There was a continuous determination of the conductivity of the aqueous medium. As a reference, the conductivity was monitored in an identical solution with the same volume, without a sample being immersed herein and in a solution into which an uncoated sample has been immersed. In an otherwise identical test series, the aqueous solutions were inoculated with bacterial cultures (Staphylococcus aureus and Pseudomonas aeroginosa). In this series of experiments, a microscopic examination was carried out after 2 weeks and after 4 weeks. The water contact angles after coating (t0), after 15 days (t15), 30 days (t30), after 45 days (t45) and after 60 days (t60) were determined. Furthermore, every 2 days, the concentrations of magnesium or iron ions in the aqueous solution were determined.

### Results:

The water contact angles determined after coating with one of the hydrophobic cationic polyelectrolytes A) or B) were between 65° and 72° for D1a, and between 85 and 96° for D1b and D1c, D3 and D3b. For D2 the contact angle was between 82° and 90° and for D2b between 92° and 98°. After the final coating with a carboxylic acid, the contact angle range rose and narrowed for coatings made with oleic acid (85-90°) and for those coated with nitro-oleic acid (96-106°). In the follow-up studies, the water contact angle first decreased by more than 5% for D1a at time point t14. Until time point t30, there was no significant change in the water contact angles measured for the remaining samples. At time t45 there was a 5% reduction in the water contact angle for the D1b and D1c series. At time point t60 there was a >5% reduction in the contact angle for D2. An increase in conductivity and the presence of magnesium or iron ions and their concentrations were correlated and were detected after a >5% reduction in water contact angles of the coated surfaces.

For the preparations stored in a bacterial culture, adherence of bacteria was detectable for D1a at t14 and t30. For D1b and D1c, there was only a small population at time t30. All other preparations showed no accumulation/colonization of bacteria.

The results for the coating substances A. and B. showed no significant differences.

### Example 6

Investigation of the Release Behavior of Compounds from Surface Coatings.

40 electropolished copper discs (2×3 cm) were cleaned with acetone and hexane and coated according to the following procedure:

1. Surface hydrophobization was achieved with dopamine (as in Example 1), 2. A 4-layer application of a mixture of compound 4 (branched PEI-C12 (50%) was prepared according to Example 2) with the dye Nile-red (0.1% by weight) or a mixture of Nile Red together with nitro-oleic acid 0.2 wt %, each dissolved in pentane, which were applied at a temperature of 40° C. by means of a spray-coating process, wherein the samples were vacuum dried at 60° C. for 12 h between the coatings. 3. For the final layer deposition nitro-oleic acid dissolved in a methanol phase was used. Then the preparations were rinsed with methanol and dried. For some of the preparations (series 1) electrical contacts were attached and connected to a DC voltage source. The samples were immersed in an electrolytic solution (NaCl) with phenolphthalein except the contact points, and a voltage of 12V was applied between the samples and the solution for 10 days. A spectroscopic analysis of the color spectrum and the intensity of coloring of the solutions to quantify leakage current was performed daily. The other half of the samples (Test Series 2) were placed in culture dishes filled with a fibroblast-containing medium and cultivated for 4 and 8 weeks. Subsequently, the cell layers were removed with trypsin and one part was examined by fluorescence microscopy and the remaining cells were lysed. The cell lysate was obtained using hexane and then the content of Nile-red in the solvent phase was determined spectroscopically. The sample surfaces were cleaned after removal of the cells with an ethanol solution in an ultrasonic bath and then dried. This was followed by water contact angle measurements.

### Results:

After the surface coating, the contact angles were between 104° and 100°.

In test series 1, there was no color change in the solutions, whereby changes in color would indicate hydrolysis, over the course of 10 days thus the coated substrates were electrically isolated without defects. The subsequently determined contact angles were virtually unchanged. The fibroblasts grown on the coated platelets had a reddish color, and light microscopy revealed numerous cytoplasmic red fluorescent vesicles. After transfer of the hydrophobic compounds into the solvent phase, Nile-red could be detected here. The vesicles were larger and the Nil-red content in the cell lysate was higher in samples coated with a mixture of Nile Red and nitro-fatty acid than when the dye alone was added to the hydrophobic cationic polyelectrolyte. The contact angles determined after detachment of the cells were between 110° and 115° after 4 weeks and between 85° and 99° after 8 weeks. Thus, a release of hydrophobic compounds, which had been introduced into a layered construction, consisting of hydrophobic cationic polyelectrolyte, which is/can be controlled by addition of a nitro-fatty acids for solubilization. The release can take place independently of the degradation of the coating.

### Example 7

### Investigation of the Degradation Delay of Bioresorbable Materials

Arterial scaffolds (S) made from a magnesium alloy (MeKo, Germany) and surgical suture material (N) made of PLA (Vicryl rapid, Ethicon, Germany) were investigated. For this purpose, the pieces of material were first cleaned in an ultrasonic bath with hexane. Coatings were made using the following arrangements, using hydrophobic cationic electrolytes and polyelectrolytes of Example 2 (corresponding experimental number):

1) PEI-C8, 90% degree of alkylation (prepared according to Example 2, compound 6), 1-layer coating with a 5% solution (pentane),

2) PEI-C12, degree of alkylation 50% (compound 5), 2-layer coating with a 5% solution, the compound for the first coating in test series was a) oleic acid and in series b) nitro-oleic acid in an amount in which the molar mass ratio between cationic groups and carboxyl groups was 1:1.5;

3) PEI-C12 (compound 2), degree of alkylation 90%, 4-layer coating, 5. PEI-C8, degree of alkylation 80% (compound 7), the compound being mixed in the test series was a) oleic acid and in series b) nitro-oleic acid provided in an amount in which the molar mass ratio between cationic groups and carboxyl groups was 1:1.2;

4. PEI-C12, degree of alkylation 90% (compound 2), wherein the substances were alternately applied for a total of 6 times. Finally, the materials were coated with a) oleic acid or b) nitro-oleic acid, each in methanol.

For control purposes, the substrate materials were each coated only with oleic acid (K1) or nitro-oleic acid (K2). Furthermore, as a reference sample, pieces of material without a surface treatment were similarly examined (Ref).

The coatings/wetting were carried out in each case by means of dip-coating for a period of 3 to 5 minutes. Subsequently, the materials were rinsed with ethanol. For each series of experiments or each test period 5 pieces of material were examined. These were individually placed in a water bath with a buffer solution (citrate/sodium carbonate, pH 7.4) for a duration of 2 (t2), 4 (t4) 6 (t6) and 8 (T8) weeks. In addition, each of 5 scaffolds was mounted on a balloon catheter and hereby expanded in a silicone tube to the nominal diameter, so that the expanded scaffold remained tightly in the silicone tube, which was then filled with the buffer solution without air. This tube was then perfused with this solution at a flow rate of 6 liters/hour for 2 weeks. Sequential images of the expanded scaffold were obtained, allowing visual analysis for erosion/fracturing of scaffold struts.

In the solutions in which the scaffolds were inserted, the magnesium content was determined daily; the solutions were replaced every 3 days. In the solutions containing the surgical suture material, the conductivity and the pH were continuously measured. The pieces of the samples were examined at the end of each experiment by light and scanning electron microscopy. The suture material was tested for tensile strength at 7 and 14 days.

### Results:

In the reference samples of S there was a steadily increasing concentration of magnesium in the storage medium after just a few hours. In K1, the increase was delayed by 14 hours and in K2 by 26 hours and the slope was less steep. For coatings 1), 2) and 3), the onset of measurable magnesium leaching was (for a)/b)): 72/95; 152/206 and 325/478 hours. The respective slope was less than that for K1 and K2 and the slope for coatings 1)-3) was lower with increasing test number. In the image analysis of expanded scaffolds, strut fracture was significantly delayed as compared to the reference sample: K1+2 days, K2+4 days, 1a)+6 days, 1b)+8 days, 2a)+12 days, 2b)+16 days, 3a)+18 days, 3b)+23 days. Also the further degradation behavior was more delayed with increasing test number as well as the duration until there was complete dissolution or removal of the material. In N (suture material), the reference sample showed an increase in conductivity after 5 hours and a pH shift after 12 hours.

In comparison to the reference sample such a change was observed in the coated samples after: K1 12/21, K2 19/28, 1 a) 27/33, 1 b) 39/47, 2 a) 41/52, 2 b) 51/60, 3 a) 70/81 and 3 b) 104/121 hours. The tensile strength was only slightly higher for K1 and K2 compared to the reference sample, but significantly higher after 7 days for materials with coatings 1-3 (a)/b): +120/+140%, +180/+201% and +350/+400%. The reference suture materials had already lost their tensile strength after 14 days, whereas the tensile strength for the samples with coatings 1 to 3 differed only slightly from the tensile strength at time t7.

### Example 8

Investigation of Formation of a Reservoir with Compounds for Biofilm Suppression

The distal ends (8 cm including the balloon) of urinary catheters (Uromed, Germany) (BK) and the shaft of venous indwelling cannulas (Braunule, BBraun, Germany) (IC) were examined. This was followed by coating with PEI-C12 with a 60% degree of alkylation according to Example 2 (compound 4). The application was carried out using a micropipetting method. After drying, a methanol solution of a) oleic acid or b) nitro-oleic acid was subsequently applied by the same procedure, in a volume amount corresponding to an order of the respective carboxylic acid in a ratio of 2:1 of the molar concentration of the carboxyl groups to that of the cationic groups of the applied polyelectrolyte. This coating sequence was repeated 4 times. Each coating sequence was completed with a coating of the carboxylic acid that has previously been added to the compound. This was followed by extensive rinsing of the material surfaces with an ethanol solution. Then the materials were dried and stored under sterile conditions.

Furthermore, the incorporation and reservoir formation of carboxylic acids in extrudable plastics was investigated. For this purpose, a polypropylene powder was soaked in a toluene solution containing 5% by weight PEI-C6 with a degree of alkylation of 50% and a) oleic acid or b) nitro-oleic acid in an amount corresponding to a 2:1 molar concentration ratio between carboxyl groups and cationic groups, under continuous mixing in a vacuum evaporator. The resulting granules were formed into thin threads (sutures) by means of an extruder. In each case 10 cm of the suture material (FM) were used for the experiments.

A bacterial culture (Staphylococcus aureus and Escheria coli) was prepared in a nutrient medium. Samples of the batch were diluted so that about 1,000 pathogens/ml were present. The coated pieces of material and uncoated reference samples were placed therein for 12 hours. Subsequently, the samples of the material were rinsed with sterile NaCl solution and left in sterile NaCl solution for 6 hours.

This process was repeated 3 times. This was followed by cell fixation and staining of the material surfaces. The storage solutions were analyzed for the presence of cells by means of a cell analyzer (Coulter Counter, Z1, Beckman, Germany) and the number of cells was determined.

Then, the IC (indwelling cannulas) were placed in a PTFE tubing flow model in which human blood serum was circulated for 5 hours. The serum was then tested for thrombin-antithombin complex (TAT) content. The relative change relative to the value measured at the starting time was determined.

The IC were then rinsed with NaCl solution and placed again in the same flow model in which a diluted platelet concentrate was circulated for 5 hours then. Thereafter, the IC were rinsed and then subjected to fluorescence staining. Fluorescence microscopy quantified adherent platelets. The experiments were also carried out with uncoated IC (IC ref).

### Results:

The reference samples of the UK had extensive bacterial lawns on all surfaces. In the storage fluids cells (bacteria) were present in numbers >200/ml at all times. No adherent bacteria could be detected in any of the coated samples. Occasionally there were cells (bacteria) in the first storage solution. No cells were present in the subsequent storage fluids. In the case of IC-Ref, there was a moderate increase in the TAT of between 230 and 280%. Furthermore, there was a nonhomogeneous adhesion of platelets, which covered about 50% of the total area. For the coated IC, the increase in TAT was 130-160%. No platelet attachments were found.

### Example 9

Investigation of the Degradation Behavior of a Coating with Hydrophobic Cationic Polyelectrolytes

The removability or degradability of the pure coating as a function of the application and the properties of the selected compounds was investigated by means of coatings on cleaned silicon discs. For this purpose, the following cationic compounds were selected:

1. Polyethyleneimine (PEI) 25 kD, average degree of branching

2. PEI, 25 kD, average degree of branching with an alkylation of 90% of the cationic groups,

3. PEI 75 kD low degree of branching with an alkylation of 80% of the cationic groups,

4. PEI, 25 kD, average degree of branching with an alkylation of 50% of the cationic groups containing nitro-oleic acid in a ratio of molar concentrations of the carboxyl groups to the non-alkylated cationic groups of 2:1.

The coating of the material surfaces took place by means of the following process designs:

A) immersion in a 5% by weight solution of the respective compounds (in the case of compound 1, the solution was prepared in ethanol) with pentane for 10 minutes, then dried; then immersed in a 5% by weight solution of nitro-oleic acid in methanol for 3 minutes, thereafter the sample was dried.

B) as well as A) were then re-immersed in the solution containing dissolved cationic polyelectrolyte (optionally with nitro-oleic acid), thereafter the samples were dried, then they were immersed in a 5% by weight solution of nitro-oleic acid in methanol for 3 minutes, thereafter the sample was dried.

C) immersion in a 5% by weight solution of the respective compounds (in the case of compound 1, the solution was prepared in ethanol) with pentane for 10 minutes, then dried; then repeated application of the solution by spin-coating, followed by drying and repetition of the spin-coating order, again followed by drying; then they were immersed in a 5% by weight solution of nitro-oleic acid in methanol for 3 minutes.

For each coating series, 10 samples were prepared.

Half of each sample was stored in different containers containing a PBS solution at 35° C., which were under slight, continuous agitation. A sample was taken once a day from each of the containers, which were dried in a stream of nitrogen. Thereafter, contact angle (20° C.) were measured at predefined locations on the samples. Then re-insert into the buffer solution of the container. On the following day, the samples of the second container were taken and analyzed in an identical manner. The experiment was carried out over 80 days.

The time (day) at which there was contact angle has decreased compared to the initial one of >5% to 10% (Δ5), >10 to 20% (Δ10), >20-50% (Δ20) and >50% (Δ50) in more than half of the samples was determined.

### Results (Numerical Values are Listed in Table 3):

The different coatings with compound 1 resulted in water contact angles between 75 and 84°. The contact angles achieved for the other coatings were between 85 and 95°. The coatings with compound 1 show a considerable variability of the contact angle measurements after only a short time and there was a rapid loss of hydrophobicity. When using the hydrophobic cationic compounds, only a small variability of the contact angle values was observed, which however varied considerably after falling below a contact angle of 50°. Depending on the layer thickness and the presence of nitro-oleic acid in a layer structure, the contact angles remained unchanged in a range which was above 80°.

### Example 10

### Investigation of Biofouling and Cell Adhesion Behavior

Surgical mesh made of polyurethane (PU) and polylactate (PLA) were used for the study. In this case, 5×5 mm pieces of solid and porous materials were cleaned and then coated with compound 1 according to Example 2 and 4 coats of method B as shown in Example 9. Linolenic acid (LA) and nitro-linolenic acid (NLA), respectively, were used as the carboxylic acids. In addition, pieces of material were coated with nitro-linolenic acid without prior application of a hydrophobic cationic polyelectrolyte by means of dip-coating (Ref-NFA). After drying, the materials were stored in a NaCl solution for 4 weeks. After re-drying, pieces of material were fixed in a Teflon vessel at 2 places in a hanging position. In parallel batches, the sample vessels were filled with the following solutions: 1. 0.9% NaCl solution, 2. human albumin 2% by weight, 3. human albumin 2% by weight with addition of fibronectin or laminin, 4. human plasma. The preparations were left here for 12 hours and then gently rinsed with NaCl solution. A set of samples was examined for protein adsorption by means of an immunofluorescence method and the degree of coverage quantified. Another set of samples was used for cell culture studies after placing the pieces of material in a culture dish and a suspension consisting of 1% FCS and cultured fibroblasts were dropwise added until the dish was completely filled. The culture dishes that were sealed were then tilted so that the area of the films was aligned in a vertical position to the horizontal. The vessels were moved slowly at 37° C. continuously on a shaker plate. Furthermore, a continuous gas exchange was ensured. Incubation was for 24, 48 and 96 hours. Subsequently, the films were gently rinsed with NaCl solution and placed in a fixation bath. Histological staining and light microscopic evaluation of the adherent cells were then performed. In addition, the concentration of TGF-β in the storage liquid was analyzed.

### Results:

For Ref-NFA, there was a homogeneous coverage of the material surface with albumin in test series 2-4.

The coverage was more pronounced when fibronectin or laminin had been present in the aqueous solution. The films coated with LA had a slightly lower surface coverage, while the material surfaces coated with NLA had virtually no surface coverage of albumin, fibronectin, or laminin.

In the light-optical analysis of the cells adhering to the material surfaces, a distinction was made as to whether cells were present at the time of addition of the cell suspension up-(order side) or down-facing side in order to estimate whether cell adhesion occurs immediately when entering the solution (application side) and/or whether this was a result of adhesion from the agitated cell suspension. After a previous incubation with NaCl solution, only a few fibroblasts were adherent to the Ref-NFA samples after 24 hours, which were present on the order side. The numbers increased after 36 h and 92 h respectively, with cell colonies having formed on the application side. There was a significant difference between the materials: in PLA, the cell count was almost twice as high as in PU. On the surfaces of the preparations which had been coated with the hydrophobic cationic polyelectrolyte together with LA, colonies with fibroblasts were present after 24 hours on the order side, which were partially confluent after 36 hours and practically completely confluent after 92 hours. Only a few fibroblasts were adherent to the undersides. On the preparations, which were coated with the hydrophobic cationic polyelectrolyte together with NLA, there was already almost complete coverage of the application side as well as the underside with fibroblasts after 24 hours. For the samples incubated with albumin or blood plasma, the Ref-NLA samples had a dense population of fibroblasts as early as 36 hours, which was more pronounced on the application side. After 92 hours surface multilayers were present on the application side that in parts spontaneously released/detached from the sample. In the case of the Ref-NLA samples incubated with laminin or fibronectin, there was a dense coating of fibroblasts, whereby in parts multiple layers had formed already after 36 hours. After 92 hours there was a thick build-up of the adherent cells on both sides. Preparations which had been coated with the hydrophobic cationic polyelectrolyte together with NLA and incubated in albumin or blood serum had a higher coverage rate of adherent cells after 36 hours compared with the preparations Ref-NFA, and also developed multilayers after 92 hours. After incubation with laminin or fibronectin, fibroblast coverage was completed earlier than in the Ref-LA samples; however, significantly fewer multilayers were formed and spontaneous detachment of cell aggregates did not occur. The cell density was about the same on both sides.

In the samples coated with the hydrophobic cationic polyelectrolyte along with NLA and incubated in albumin or serum, a uniform population of fibroblasts that was equally distributed on both sides of the preparations was present even after 24 hours. In the further course, it came only occasionally to a multilayer formation. There was no detachment of cell aggregates. For the NLA samples incubated with laminin or fibronectin there was virtually no difference in cellularity as compared to preparations incubated with albumin or serum alone. In particular, there was no multilayer formation.

In representative sections/areas, the cell geometry was assessed by light microscopy. It showed that cells adhering to the Ref-NLA samples had a flattened shape after 36 hours, provided that the material consisted of PU but had a predominantly dendritic shape in PLA materials. Cells adhering to Ref-NLA samples that had been conditioned with albumin or serum were predominantly dendritic in shape, and exclusively dendritic in shape when conditioned with fibronectin or elastin. For samples coated with the hydrophobic cationic polyelectrolyte along with LA, the adherent cells had a globular shape except after incubation with laminin or fibronectin (here flattened to dendritic forms were present). Samples coated with the hydrophobic cationic polyelectrolyte along with NLA had a spherical cell shape at all times.

The TGF-β concentration in the respective storage medium in the Ref-NLA samples generally correlated with the number of adherent fibroblasts. However, significantly higher levels were recorded when the material was PLA or incubated with fibronectin or laminin. The TGF-β levels measured on the samples coated with the hydrophobic cationic polyelectrolyte along with LA were always significantly below those measured for the Ref-NLA samples. The highest values were measured on samples which had been incubated with fibronectin or laminin. For the samples coated with the hydrophobic cationic polyelectrolyte along with NLA, the TGF-β levels were significantly lower than for the samples coated with the hydrophobic cationic polyelectrolyte along with LA. In contrast to other experimental conditions there was also no increase in TGF-β concentration when the samples were incubated with fibronectin or laminin.

### Example 11

### Investigation of the Influence of Implant Material on Surface Contact Activation

Here, 1 cm sized pieces of textured silicone film (PolyTech, Germany) that are used for making silicone implants were subjected to the following coating procedures: 1. no surface treatment (reference), 2. nitro-oleic acid; 3. hydrophobic cationic polyelectrolyte according to compound 1 in example 2, which is consecutively applied 3 times according to procedure A in example 9 with a) oleic acid or b) nitro-oleic acid; 4. hydrophobic cationic polyelectrolyte according to compound 1, example 2, which is applied 4 times according to procedure C in example 9 with a) oleic acid or b) nitro-oleic acid.

The preparations are stored in a PBS solution at 25° C. for 3 weeks. Thereafter, the preparations were copiously rinsed and dried. Two sets of preparations were placed individually in micro-reaction tubes and incubated with human serum for 1 hour at 37° C. Two additional sets were incubated with a NaCl solution. After removal from the incubation solutions, one set of the samples was placed in a NaCl solution for 5 minutes and then rinsed off with it. Then, the samples were removed and rinsed to remove the incubation solutions; thereafter they were placed in separate reaction vessels. Human mononuclear cells obtained from healthy individuals' blood were suspended in a nutrient medium and added to the reaction vessels. These were incubated for 3 days under standard culture conditions. Subsequently, the supernatant was analyzed for the content of IL-1beta, IL-6, IL-8, and chemoattractant protein-1 (MCP-1).

After incubation, one set of samples was used for immunofluorescent staining to detect fibrinogen and the monocyte-adhesion complex C5b-9. Quantification was carried out by means of fluorescence microscopy.

### Results:

The reference samples showed extensive protein adsorption. With preparations 2-4 no adherent proteins could be detected. In principle, identical results were found for the detection of fibrinogen. Monocyte-adhesion complexes C5b-9 were only detected in the reference samples.

After incubation of the reference samples which had been incubated with a NaCl solution and then with mononuclear cells, there was a marked increase in IL-8 and MCP-1, as well as a moderate increase of IL1-beta and IL 6 in the culture medium. The reference samples, which were incubated in serum and then cultured in the suspension with mononuclear cells, showed a very strong increase in the investigated cytokines.

In the preparations 2-4, in which oleic acid had been used for reservoir formation as well as for surface coating and in which incubation was carried out by means of a NaCl solution, there was practically no increase in the measured cytokines compared to the baseline measurement. In these preparations, however, there was a moderate increase in IL8 and IL6 after incubation in serum. MCP1 and IL1-beta were only minimally elevated. In preparations which were loaded during the coating construction or provided with surface coating with nitro-oleic acid, the cytokine level increases were never greater than 10% of the initial level at any time during the experiment.

### Example 12

### Investigation of the Adhesion and Proliferation Behavior of Monocytes and Fibroblasts on Surgical Sutures.

A 1 mm diameter suture material was extruded from a polypropylene granulate (Mat. 1). Furthermore, the granules were mechanically crushed to obtain a coarse powder. It was mixed with a hydrophobic cationic electrolyte (compound 1, according to Example 2) and 13-nitro-cis-13-docosenoic acid (nitro-erucic acid, NE). The application was carried out under continuous mixing 2 times. After evaporation of the solvent phase, yellow-brown granules had formed, whereby 3% by weight of the compound mixture had been introduced into the polypropylene starting material. A suture was then extruded (Mat. 2) as previously described. Furthermore, suture of Mat.1 were surface-coated with hydrophobic cationic polyelectrolytes: Compound 1 according to Example 2, Compound 2 according to Example 2. This suture material is referred to as Mat.-3.

Furthermore, strips with a width of 2 mm were prepared from a PTFE film (material thickness 100 μm), which was coated as Mat-YY (3). Both the coated PTFE strips (Mat. 4) and the uncoated PTFE strips (Mat. 5) were used for the tests.

After drying the thread materials were stored in an inert gas atmosphere. To carry out the experiment, the suture materials were placed in a flat-bottomed culture vessel, which had a clamping device made of PTFE attached to the base and which allowed fixation of the thread ends and tension of the threads, so that they were in an extended form and did not touch the bottom of the container.

Human umbilical venous endothelial cells (HUVEC), human vascular smooth muscle cells (SMC) and mouse fibroblasts (MF) were cultured and suspended in a nutrient medium (FCS 5%).

The culture vessels were each filled with one of the cell suspensions, so that the filaments were completely covered even during agitation of the medium, which was carried out during the further cultivation under standard cultivation conditions. The studies were carried out in a 6-fold parallel experimental setup for a period of 2 (T2), 4 (t4), 6 (t6) and 8 weeks (t8). At the end of each experiment, the suture materials were taken out and gently rinsed and incubated with methylene blue for incident light in situ microscopy, and also with calcein AM and a propidium iodide solution. Fluorescence microscopy was then performed.

Furthermore, filaments were subjected to scanning electron microscopy. In one set of samples, the preparations were placed in a trypsin solution and finally in an ultrasonic bath. Then REM was performed.

### Results:

On Mat. 5, only a few living and mostly isolated cells were found at all time points. The morphology of the fibroblasts was polygonal. On Mat. 3, numerous MF were deposited at T2 with a polygonal shape and to a lesser extent SMC (<10% of the total area), whereby practically no endothelial cells practically adhered. The number of cells increased over the course of the examination and resulted in a total coverage area of the fibroblasts of about 45% of the total surface area at time t8. The relative coverage rate for SMC at t8 was about 20% and for HUVEC <10%. In contrast to the uncoated materials, the coated materials were rapidly covered with all investigated cell lines. This was predominantly complete (>90% of the total area) for HUVEC at t30 for Mat.2-4, for SMC at t2 at Mat.2 and Mat.-3, at t4 for Mat.-4 and for fibroblasts at t14 at Mat-2-4. The cells had a flattened or rounded shape in a full-surface cell cluster. In the further course, multiple layers of the cell aggregates did not form. In living/death staining, alive and dead cells were found at equal frequency on Mat. 5. On Mat.-1, the ratio between living and dead cells present on the material surface was between 15 and 25% for the various cell lines up to t4, and subsequently the ratio decreased to between 5 and 15% at t8. There were only sporadically cells adhering to Mat.-2-Mat.-4 (<5% of cells) until t2. From this point on, only living cells were detected. In REM, the fibroblasts and SMC adhering to the material surface of Mat.-1 had a dendritic or spherical shape (esp. in HUVEC). On the surface of Mat. 5, until t2, only dendritic fibroblasts and SMC were present. In the process, a spindle-shaped cell morphology occurred in about 50-60% of the cells, the remaining cells remained in dendritic form. In contrast, dendritic cell forms of SMC or fibroblasts were only occasionally present at time t2, and the majority of the cells had a spindle-like shape.

From time t4, all cells were flattened and had a planar spread in a closed cell composite. After detachment of the cells (no assessment for Mat.-5), Mat.-1 showed a coarsely pored collagen network with clear level differences at time t8. In Mat. 2-4 a fine-reticular collagen network that had a very uniform surface boundary was found.

### Example 13

### Process for Surface Coating of Implant Materials

For the investigations, medical planar silicone material (PolyTech Health, Germany) (S), which is used for the production of soft tissue implants, and stainless steel 316 in the form of thin discs, were used. All material surfaces were cleaned in an ultrasonic bath with methanol and diethyl ether and then dried.

For the coatings, the following hydrophobic cationic polyelectrolytes were prepared according to Example 2 with the following experimental numbers:

1. Compound 1 according to Ex. 2, dissolved in methanol

2. Compound 1 according to Ex. 2, dissolved in THF

3. Compound 1 according to Ex. 2, dissolved in toluene

4. Compound 1 according to Ex. 2 dissolved in pentane

5. Compound 3 according to Ex. 2, dissolved in pentane

6. Compound 7 according to Ex. 2 dissolved in pentane

Solvent mixtures having a 5 and 10% concentration of the respective compounds or compound mixtures were used.

The coatings were carried out by means of the following application methods:

A) Dipping coating carried out by immersing the sample in the solvent mixture for 10 to 60 seconds.

B) Spray coating carried out by a spray device which ensured a full-surface application of >50% of the volume of the solvent mixture used.

C) Micropipetting method, performed with a manual pipetting, which ensured application of >80% of the solvent mixture used.

The adding or application with one or more of the following carboxylic acids was carried out for reservoir formation and/or implementation of process step d):

a) oleic acid

b) stearic acid

c) linoleic acid

d) nitro-oleic acid

e) nitro-stearic acid

f) nitro-linoleic acid

Surface Coatings were Made According to the Following Procedure:

1. Surface coating with compounds 1-6, whereby each of which is used for one coating, in each of the application methods A) to C). Thereafter drying the material surfaces.

Subsequently, a coating with one of the carboxylic acids/nitro-carboxylic acids a) to f) was carried out for each of the used hydrophobic cationic electrolytes or polyelectrolytes, and application methods. Then the preparations were rinsed with ethanol and the material surfaces dried.

2. Surface coating with compounds 1-6, whereby each of which is used for one coating, in each of the application methods A) to C). Thereafter the material surfaces are dried. Subsequently, the same hydrophobic cationic electrolytes or polyelectrolytes was applied again with the coating procedure with the application process B) or C). Thereafter the material surfaces were dried. Subsequently, a coating with one of the carboxylic acids/nitro-carboxylic acids a) to f) was carried out for each of the related hydrophobic cationic electrolytes or polyelectrolytes, as well as application methods. Then the preparations were rinsed with ethanol and the material surfaces were dried.

3. Surface coatings comprising compounds 1 and 2, whereby each of which is used for one coating, in each of the application methods A) to C). Thereafter the material surfaces were dried. Subsequently, a further coating with one of the hydrophobic cationic electrolytes or polyelectrolytes of Nos. 3 to 6 was carried out with each of the coated material pieces using application method A) or C). Thereafter the material surfaces were dried. Subsequently, a coating with one of the carboxylic acids/nitro-carboxylic acids a) to f) was carried out for each of the related hydrophobic cationic electrolytes or polyelectrolytes, as well as application methods. Then the preparations were rinsed with ethanol and the material surfaces dried.

4. Surface coatings with compounds 5. and 6., whereby each of which is used for one coating, in each of the application methods A) to C). Thereafter drying the material surfaces were dried. Subsequently, a further coating with one of the hydrophobic cationic electrolytes or polyelectrolytes of the numbers 1 and 2 was carried out with each of the coated material pieces using application method A) or C). Thereafter the material surfaces were dried. Subsequently, a coating with one of the carboxylic acids/nitro-carboxylic acids a) to f) was carried out for each of the related hydrophobic cationic electrolytes or polyelectrolytes, as well as application methods. Then the preparations were rinsed with ethanol and the material surfaces dried.

5. Surface coatings comprising compounds 1 and 2, whereby each of which is used for one coating, in each of the application methods A) to C), respectively. Thereafter the material surfaces were dried. Subsequently, a coating with one of the carboxylic acids/nitro-carboxylic acids a) to f) was carried out for each of the used hydrophobic cationic electrolytes or polyelectrolytes, as well as application methods. Subsequently, a further coating with one of the hydrophobic cationic electrolytes or polyelectrolytes of the numbers 5 and 6, respectively, was carried out with the coated material pieces using the application method A) or C). Thereafter the material surfaces were dried. Subsequently, for each of the used hydrophobic cationic electrolytes or polyelectrolytes and application methods, a coating was carried out with one of the carboxylic acids/nitro-carboxylic acids a) to f) previously used. Then the preparations were rinsed with ethanol and the material surfaces dried.

6. Surface coatings containing compounds 5 and 6, respectively, were produced, each with one of the carboxylic acids or nitro-carboxylic acids according to a) to f) with a ratio of the molar concentrations of the carboxylic acid residues to cationic groups of the hydrophobic polyelectrolytes of 2:1 added and mixed with these and the resulting mixtures are each used for coating, using application methods A) to C), respectively. Thereafter the material surfaces are dried. Subsequently, a further coating with one of the hydrophobic cationic electrolytes or polyelectrolytes of the numbers 1 and 2 was carried out in each case with the coated material pieces, using the application method A) or C). Thereafter the material surfaces were dried. Subsequently, a coating with one of the carboxylic acids/nitro-carboxylic acids a) to f) was carried out for each of the related hydrophobic cationic electrolytes or polyelectrolytes, as well as application methods. Then the preparations were rinsed with ethanol and the material surface dried.

Repeated water contact angle measurements were made at various points on all material surfaces. The coated pieces of material were placed in a NaCl or a PBS solution over a period of 4 weeks. Storage was carried out at a temperature of 37° C. under slight agitation of the aqueous medium. Then the pieces of material were removed and rinsed thoroughly with ion-free water. Then the material samples were dried and the water contact angles were measured as previously described.

### Results:

The contact angles of the starting material were 112° for S and 42° for E.

After coating the contact angle were between 82° and 98°. After being placed in an aqueous solution for 4 weeks, the specific contact angles ranged between 85° and 104°. In the direct comparison of the individual material surfaces, there was no change in the contact angle by >5% over the course of time.

### Example 14 Solubility Studies

Reaction 2: Reaction of PEI-C12 (100% or 80%, respectively) and PEI-C8 (80%) from Example 2 with oleic acid.

For the conversion into the oleate form of the resulting product, a ratio of 2.1 mL (6.61 mmol) oleic acid per gram of PEI-C12 is used (1 g, 43 μmol).

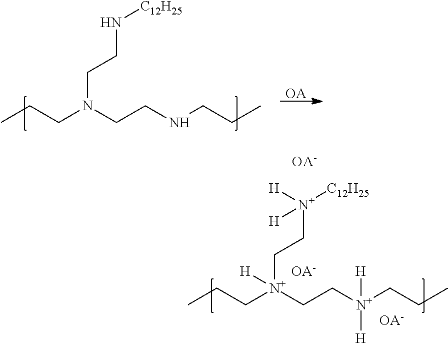

First, 3.87 g (0.17 mmol) PEI-C12/OA (M ˜23,263 g/mol) is placed in a 50 mL centrifuge tube and dissolved in 20 mL toluene with the aid of an ultrasonic bath and occasional stirring.

Thereafter, 8.12 mL/7.23 g (25.6 mmol) of oleic acid dissolved in 20 mL of toluene is added with stirring to the pale yellow solution.

The solution is allowed to stand overnight. The solution turns slightly dark yellow. The solution is transferred to a 100 mL round bottom flask and the solution is concentrated strongly on a rotary evaporator. The solution must be concentrated to such an extent that it can be transferred by means of a pipette into a 50 mL centrifuge tube. The residual solvent is carefully removed in vacuo. Subsequently, the viscous mass is washed three times with methanol to remove excess oleic acid. The remaining methanol is first extracted in vacuo for 12 h at RT and then for 12 h at 70° C. In this case, a viscous amber-colored mass is obtained.

To determine a suitable solution for coating, the solubility of the amino form (PEI-C12 (80%)) and the oleate form (PEI-C12 (80%)/OA), (PEI-C12 (100%)) and the oleate form (PEI-C12 (100%)/OA) as well as (PEI-C8 (80%)/OA), (PEI-C8 (100%)) and the oleate form (PEI-C12 (100%)/OA) in different solvents was tested in various solvents.

| | Solubility | | | | | |
|---|---|---|---|---|---|---|
| | | PEI- | | PEI- | | PEI- |
| | PEI- | C12(100%)/ | PEI- | C8(80%)/ | PEI- | C12(80%)/ |
| Solvent | C12(100%) | OA | C8(80%) | OA | C12(80%) | OA |
| Water | −− | −− | −− | −− | −− | −− |
| MeOH | − | −− | +++ | − | ++ | − |
| EtOH | ++ | − | ++ | − | ++ | − |
| Iso-propanol | + | − | + | N/A | + | −− |
| DMSO | ++ | − | + | −− | + | −− |
| THF | +++ | +++ | +++ | +++ | +++ | +++ |
| TBME | ++ | ++ | +++ | + | − | N/A |
| Acetone | − | −− | − | N/A | −− | −− |
| Ethylacetate | −− | −− | N/A | N/A | − | − |
| DCM | ++ | N/A | N/A | N/A | + | ++ |
| Toluene | ++ | ++ | N/A | N/A | ++ | ++ |
| Pentane | +++ | +++ | − | +++ | − | +++ |
| Iso-octane | +++ | +++ | N/A | N/A | ++ | +++ |
| Diethylether | N/A | N/A | + | + | + | ++ |
| CHCl₃ | N/A | N/A | +++ | +++ | + | ++ |

(−) insoluble; (−) poorly soluble; (+) moderately soluble; (++) well soluble; (+++) very soluble; NA: not available.

It can be seen that PEI-C8 (80%) is well soluble in THF but poorly soluble in pentane. In contrast, PEI-C12 (100%) is very soluble in pentane. For example, it was also possible to show that an coverage of a material surface with PEI (25 kD/middle degree of branching), in which 80% alkylation was carried out with a C-8 alkane, which dissolved well in THF but poorly in pentane and as 5% solution by means of dip coating over 30 min was deposited, and after drying a 5% solution of the same starting compound, but with a degree of alkylation of 100% with the same alkylating agent, which was dissolved in pentane and this mixture was applied by means of a micropippetting method in a defined amount on the material surface, it was found by the determinations of the weight of the material which took place at the end and after each coating time that a summative order/deposition of the compounds was achieved.

What is claimed is:

1.-34. (canceled)

35. A biodegradable surface coating of a medical product, wherein the surface coating is a carboxylic acid layer comprising at least one carboxylic acid having a carbon chain length from 6 to 24; and at least one hydrophobic cationic polyelectrolyte, wherein the hydrophobic cationic polyelectrolyte as well as the carboxylic acid is applied from an anhydrous solvent mixture, wherein the carboxylic acid layer is located on the electrolyte layer, wherein the term anhydrous means that the at least one hydrophobic cationic polyelectrolyte to be applied or the at least one carboxylic acid and their solutions at most has a total amount of water of at most 1,000 ppm.

36. The surface coating according to claim 35 for at least one of corrosion and degradation delay, wherein the surface coating is at least one of insulating and self-healing.

37. The surface coating according to claim 35, wherein the surface coating has a layer thickness of 5 nm to 50 microns.

38. The surface coating according to claim 35, wherein the hydrophobic cationic electrolyte or the hydrophobic cationic polyelectrolyte is a carbon-containing compound having a molecular weight of between 200 and 500,000 Da carrying at least two cationic charge groups or at least two basic groups which are ionizable and has a partition coefficient between an octanol and a water phase in a non-ionized state K_{ow} of >0.3.

39. The surface coating according to claim 35, wherein the hydrophobic cationic polyelectrolyte carries at least two cationic charge groups or at least two basic groups and is a carbon-containing quaternary nitrogen compound, wherein the hydrophobic cationic polyelectrolyte has been rendered hydrophobic.

40. The surface coating according to claim 35, wherein the hydrophobic cationic polyelectrolyte bears at least two cationic charge groups or at least two basic groups selected from the list comprising or consisting of ammonium, guanidinium, amidinium, imidazolium, pyrrolidinium, pyrazinium, piperidinium, pyrimidinium, pyridazinium, pyrazolium, isoquinolinium, quinolinium, purinium, benzimidazolium, thiazolinium, oxazolinium, phosphonium and sulfonium.

41. The surface coating according to claim 35, wherein the hydrophobic cationic polyelectrolyte comprises polyphenylyalanine, polylysine, polyarginine, polyornithine, polyhistidine or a compound containing or consisting of amino acids lysine, arginine, histidine, phenylalanine and/or ornithine wherein said cationic polyelectrolyte is hydrophobized.

42. The surface coating according to claim 35, wherein the at least one carboxylic acid is a nitrated fatty acid.

43. The surface coating according to claim 35, wherein the water contact angle at 20° C. is ≥80°.

44. A medical product with the surface coating according to claim 35.

45. The medical product according to claim 44, wherein the medical product is an arterial stent.

46. A method for producing a surface coating for a medical product according to claim 1 comprising the following steps:
a) providing a solid material with a cleaned and/or hydrophobized material surface,
b) wetting of the surface of the solid material under anhydrous conditions with at least one hydrophobic cationic polyelectrolyte or a mixture comprising at least one hydrophobic cationic electrolyte and at least one hydrophobic cationic polyelectrolyte,
c) drying the surface,
d) wetting of the surface under anhydrous conditions with at least one carboxylic acid having a carbon chain length from 6 to 24,
e) rinsing and drying the surface,
f) obtaining the surface coating.

47. The method according to claim 46, wherein in step b) fatty acids are mixed with the at least one hydrophobic cationic polyelectrolyte and applied and/or applied separately in sequential order to produce formation of a reservoir for fatty acids and/or to dissolve supportive and/or active compounds and to integrate them into the layer structure.

48. The method according to claim 46, wherein the solid materials of step a) are corrodible and/or degradable materials.

49. The method according to claim 46, wherein the at least one hydrophobic cationic polyelectrolyte has a partition coefficient between an octanol and a water phase in a non-ionized state K_{ow} of >0.3.

50. The method according to claim 46, wherein steps b) and c) are repeated two or more times consecutively after step c) and before step d).

51. The method according to claim 46, wherein between steps c) and d) the following steps b2) and c2) are carried out:
b2) wetting of the surface under anhydrous conditions with at least one hydrophobic anionic electrolyte and/or at least one hydrophobic anionic polyelectrolyte or a mixture comprising at least one hydrophobic anionic electrolyte and at least one hydrophobic anionic polyelectrolyte;
c2) drying the surface.

52. The method according to claim 46, wherein the carboxylic acids in step d) are nitro-fatty acids having a carbon chain length of 6 to 24.

53. The method according to claim 46, wherein in step b) further at least one supportive, at least one active compounds or a mixture containing at least one supportive and at least one active compound for wetting the surface of step a) is used.

54. The method according to claim 46, wherein in step b2) at least one supportive, at least one active compounds or a mixture containing at least one supportive and at least one active compound for wetting the surface is also used.

55. The method according to claim 46, wherein the solid material is an arterial implant.

56. The method according to claim 46, wherein the hydrophobic cationic polyelectrolyte is a carbon-containing compound having a molecular weight of between 200 and 500,000 Da carrying at least two cationic charge groups or at least two basic groups which are ionizable and has a partition coefficient between an octanol and a water phase in a non-ionized state K_{ow} of >0.3.

57. The method according to claim 46, wherein the hydrophobic cationic polyelectrolyte carries at least two cationic charge groups or at least two basic groups and is a carbon-containing quaternary nitrogen compound.

58. The method according to claim 46, wherein the hydrophobic cationic polyelectrolyte carries at least two cationic charge groups or at least two basic groups selected from the list consisting of or consisting of ammonium, guanidinium, amidinium, imidazolium, pyrrolidinium, pyrazinium, Piperidinium, pyrimidinium, pyridazinium, pyrazolium, isoquinolinium, quinolinium, purinium, benzimidazolium, thiazolinium, oxazolinium, phosphonium and sulfonium.

59. The method according to claim 46, wherein the hydrophobic cationic polyelectrolyte polyphenylalanine, polylysine, polyarginine, polyornithine, polyhistidine or a compound containing or consisting of the amino acids phenylalanine, lysine, arginine, histidine and/or ornithine said cationic polyelectrolyte is rendered hydrophobic.

60. A medical product with a surface coating obtainable by a process according to claim 46.
